(19) United States
(12) Patent Application Publication
(10) Pub. No.: US 2026/0146046 A1
(43) Pub. Date: May 28, 2026

(54) BICYCLIC FERROPORTIN INHIBITORS

(71) Applicant: Vifor (International) AG, St. Gallen (CH)

(72) Inventors: Wilm Buhr, Konstanz (DE); Klaus-Daniel Umland, Winterthur (CH); Aris Kalogerakis, Dulliken (CH); Vania Manolova, Zollikon (CH); Anna Flace, Zürich (CH)

(73) Assignee: Vifor (International) AG, St. Gallen (CH)

(21) Appl. No.: 19/122,675

(22) PCT Filed: Oct. 19, 2023
(86) PCT No.: PCT/EP2023/079147

§ 371 Date: Apr. 18, 2025

(30) Foreign Application Priority Data
Oct. 21, 2022 (EP) 22202984.5

(51) Int. Cl. C07D 498/04; A61K 31/12; A61K 31/16; A61K 31/198; A61K 31/4196; A61K 31/426; A61K 31/4412; A61K 31/444; A61K 31/496; A61K 31/5377; A61K 31/7036; A61K 31/7056; A61K 31/7088; A61K 38/13; A61K 38/14; A61K 38/21; A61K 45/06; C07D 519/00

(52) U.S. Cl. CPC C07D 498/04; A61K 31/12; A61K 31/16; A61K 31/198; A61K 31/4196; A61K 31/426; A61K 31/4412; A61K 31/444; A61K 31/496; A61K 31/5377; A61K 31/7036; A61K 31/7056; A61K 31/7088; A61K 38/13; A61K 38/14; A61K 38/212; A61K 45/06; C07D 519/00

(57) ABSTRACT

The invention relates to novel compounds of the general formula (I-A) as defined in the application and pharmaceutical compositions comprising them and the use thereof as medicaments, in particular for the use as ferroportin inhibitors, more particularly for the use in the prophylaxis and/or treatment of diseases caused by a lack of hepcidin or iron metabolism disorders leading to increased iron levels or increased iron absorption, and/or iron overload.

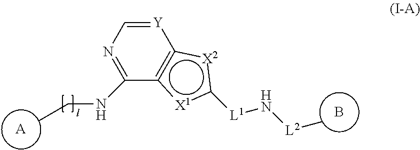

The invention relates to novel compounds of the general formula (I-A)

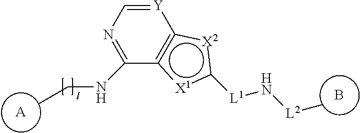

and pharmaceutically acceptable salts thereof. The compounds of the general formula (I-A) of the present invention act as ferroportin inhibitors. The novel compounds are particularly suitable for the use as medicaments in the prophylaxis and/or treatment of diseases caused by a lack of hepcidin or of iron metabolism disorders leading to increased iron levels or increased iron absorption. The compounds of the general formula (I-A) of the present invention are further particularly suitable for the use in the prophylaxis and/or treatment of iron overload, including thalassemia, sickle cell disease and hemochromatosis, as well as for the use in the prophylaxis and/or treatment of diseases related to or caused by increased iron levels, increased iron absorption or iron overload.

### BACKGROUND AND PRIOR ART

Iron is an essential trace element for almost all organisms and is relevant in particular with respect to growth and the formation of blood. The balance of the iron metabolism is in this case primarily regulated on the level of iron recovery from haemoglobin of ageing erythrocytes and the duodenal absorption of dietary iron. The released iron is taken up via the intestine, in particular via specific transport systems (DMT-1, ferroportin), transferred into the blood circulation and thereby conveyed to the appropriate tissues and organs (transferrin, transferrin receptors).

Mammalian organisms are unable to actively discharge iron. The iron metabolism is substantially controlled by hepcidin, a peptide hormone produced in the liver, via the cellular release of iron from macrophages, hepatocytes and enterocytes. Hepcidin acts on the absorption of iron via the intestine and via the placenta and on the release of iron from the reticuloendothelial system. In the body, hepcidin is synthesized in the liver from what is known as pro-hepcidin, pro-hepcidin being coded by the gene known as the HAMP gene. The formation of hepcidin is regulated in direct correlation to the organisms iron level, i.e. if the organism is supplied with sufficient iron and oxygen, more hepcidin is formed, if iron and oxygen levels are low, or in case of increased erythropoiesis less hepcidin is formed. In the small intestinal mucosal cells and in the macrophages hepcidin binds with the transport protein ferroportin, which conventionally transports the phagocytotically recycled iron from the interior of the cell into the blood.

The transport protein ferroportin is a transmembrane protein consisting of 571 amino acids which is formed in the liver, spleen, kidneys, heart, intestine and placenta. In particular, ferroportin is localized in the basolateral membrane of intestinal epithelial cells. Ferroportin bound in this way thus acts to export the iron into the blood. In this case, it is most probable that ferroportin transports iron as Fe²⁺. If hepcidin binds to ferroportin, ferroportin is transported into the interior of the cell, where its breakdown takes place so that the release of the phagocytotically recycled iron from the cells is then almost completely blocked. If the ferroportin is inactivated, for example by hepcidin, so that it is unable to export the iron which is stored in the mucosal cells, the stored iron is lost with the natural shedding of cells via the stools. The absorption of iron in the intestine is therefore reduced, when ferroportin is inactivated or inhibited, for example by hepcidin. In addition, ferroportin is markedly localized in the reticuloendothelial system (RES), to which the macrophages also belong. On the other hand, if the serum iron level decreases, hepcidin production in the hepatocytes of the liver is reduced so that less hepcidin is released and accordingly less ferroportin is inactivated, allowing a larger amount of stored iron to be transported into the serum.

Therefrom it becomes apparent that the hepcidin-ferroportin system directly regulates the iron metabolism and that a disorder of the hepcidin regulation mechanism therefore has a direct effect on iron metabolism in the organism. In principle the hepcidin-ferroportin regulation mechanism acts via the two following opposite principles:
On the one hand, an increase of hepcidin leads to inactivation of ferroportin, thus blocking the release of stored iron from the cells into the serum, thus decreasing the serum iron level. In pathological cases a decreased serum iron level leads to a reduced hemoglobin level, reduced erythrocyte production and thus to iron deficiency anemia.

On the other hand, a decrease of hepcidin results in an increase of active ferroportin, thus allowing an enhanced release of stored iron and an enhanced iron uptake e.g. from the food, thus increasing the serum iron level. In pathological cases an increased iron level leads to iron overload.

Iron overload states and diseases are characterized by excess iron levels. Therein, the problems arise from excess serum iron level which lead to non-transferrin bound iron (NTBI). The NTBI is rapidly taken up unspecifically by the organs, leading to an accumulation of iron in tissue and organs. Iron overload causes many diseases and undesired medical conditions, including cardiac, liver and endocrine damage. Further, iron accumulation in brain has been observed in patients suffering from neurodegenerative diseases such as for example Alzheimer's disease and Parkinson's disease. As a particular detrimental aspect of excess free iron the undesired formation of radicals must be mentioned. In particular iron(II) ions catalyze the formation (inter alia via Fenton reaction) of reactive oxygen species (ROS). These ROS cause damage to DNA, lipids, proteins and carbohydrates which has far-reaching effects in cells, tissue and organs and is well known and described in the literature to cause the so-called oxidative stress.

Besides the conventional methods for treating iron overload by removing iron from the body e.g. with chelating agents such as deferoxamine (also known as desferrioxamine B, N′-{5-[acetyl(hydroxy)amino]pentyl}-N-[5-({4-[(5-aminopentyl)(hydroxy)amino]-4-oxobutanoyl}amino)pentyl]-N-hydroxysuccinamide or Desferal®), deferasirox (Exjade®, 4-(3,5-bis(2-hydroxyphenyl)-1H-1,2,4-triazol-1-yl)benzoic acid) and deferiprone (Ferriprox®, 3-hydroxy-1,2-dimethylpyridin-4(1H)-one), compounds acting as hepcidin agonists or having an inhibiting or supporting effect on the biochemical regulatory pathways in the iron metabolism, such as hepcidin mimetic peptides have been described. Said therapeutic approaches are based on a direct involvement into the disturbed iron metabolism pathway by directly acting via the primary regulator hepcidin by providing a hepcidin mimetic or a hepcidin agonist, i.e. acting in the sense of a kind of hepcidin substitute or supply. The approach is based on the therapeutic rationale to treat iron overload, i.e. excess serum iron level, by inhibiting ferroportin, via the hepcidin-inactivation mechanism, thus blocking excessive iron absorption.

Ferroportin inhibitors and methods for preparing the same have been described in WO2017/068089, in WO2017/068090, in WO2021/191202, and in WO2022/223689 and WOW02023/046664. Further, the international application WO2018/192973 describes the preparation and crystallization of various specific salts of selected ferroportin inhibitors described therein and as described in WO2017/068089 and in WO2017/068090. The international applications WO2021/013771, WO2021/013772, WO2021/078889 and WO2022/157185 relate to selected Ferroportin inhibitors as described in the aforementioned applications in specific medical use applications, such as for treating transfusion dependent thalassemia, for treating acute kidney injury, for treating sickle cell disease and for treating myelodysplastic syndrome.

WO2020/123850A1 describes further ferroportin inhibitors with a broadly defined central heteroaryl bicyclic ring structure and broad variety of possible substitution. The compounds of the present invention relate to selected ferroportin inhibitors with a selected central heteroaryl bicyclic ring structure and a specifically selected substitution on the terminal groups “A” and “B”.

### OBJECT OF THE INVENTION

The object of the present invention was to provide new therapeutically effective compounds that can be used for an effective therapy for the prophylaxis and treatment of iron metabolism disorders which are associated with increased iron levels, such as in particular iron overload. In a further object, the new compounds should exhibit high efficacy in the indication of the present invention, exhibit few side effects and have a low toxicity and good bioavailability and compatibility. Moreover, these new compounds, in contrast to the known iron chelating compounds, should be suitable to prevent the occurrence of increased iron levels and thus the related disorders, instead of removing excess iron from the body when the iron overload has already occurred. In a further object the new compounds should have a defined structure (stoichiometry) and should be preparable by simple synthesis processes, exhibit less sensitivity and improved long-lasting efficiency as compared to the known biomolecular compounds, such as antibodies.

This goal was achieved by the development of the novel compounds as defined herein, such as in particular according to formula (I-A) and (I-B), which have been found to act as ferroportin inhibitors. Therewith, the novel compounds are suitable for the use in the inhibition of iron transport, and thus are effective in the prophylaxis and treatment of iron metabolism disorders which are associated with increased iron levels, such as in particular iron overload, as well as in in the prophylaxis and treatment of diseases caused by a lack of hepcidin, diseases related to or caused by increased iron levels or iron overload and diseases associated with ineffective erythropoiesis.

### DETAILED DESCRIPTION OF THE INVENTION

The inventors have found that specific compounds having the general structural formula (I-A) or (I-B) as defined herein, act as ferroportin inhibitors, thus effectively inhibiting iron transport and accordingly being particularly suitable for the use as medicaments, in particular for the use in the treatment and/or prophylaxis of diseases caused by a lack of hepcidin, diseases associated with ineffective erythropoiesis or iron metabolism disorders leading to increased iron levels, such as particularly iron overload states such as in particular thalassemia and hemochromatosis. Very particularly the new compounds turned out to be suitable for treating thalassemia and hemochromatosis. The new compounds are also suitable for the treatment of diseases caused by pathologically low hepcidin-levels and for the use in the inhibition of iron transport. In particular, the new compounds described herein show good metabolic stability and good bioavailability, which makes them particularly suitable as drug compounds.

Accordingly, the invention relates to novel compounds of general formula (I-A)

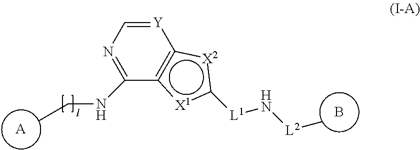

wherein
I is an integer of 1 or 2;
L¹ and L² each represent a linker group comprising 1 to 7 carbon atoms and which are independently selected from
   a linear C₁-C₃-alkyl group —[CH₂]ₘ or —[CH₂]ₙ—, respectively, wherein m and n are independently an integer of 1, 2 or 3,
   a branched C₁-C₄-alkyl group, and
   a C₃-C₆-cycloalkyl group, which forms a ring together with the nitrogen atom to which it is bonded;
X¹ is N, S or O; and
X² is N, S, or O; with the proviso that one of X¹ and X² is N;
and wherein
Y is N or CR⁵;
wherein
R⁵ represents
   H,
   halogen,
   linear or branched C₁-C₃-alkyl, or
   linear or branched C₁-C₃-haloalkyl;

A represents a group (a-1)

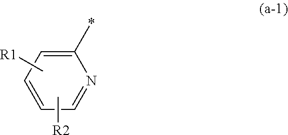

wherein * indicates the binding position;
R¹ and R² independently represent
   hydrogen
   halogen,
   linear or branched C₁-C₃-alkyl,
   linear or branched C₁-C₃-haloalkyl, or
   linear or branched C₁-C₃-alkoxy;
B represents one of the following groups (b-1), (b-2) and (b-3)

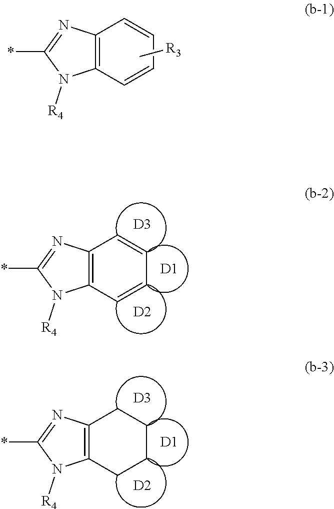

wherein * indicates the binding position;
R³ represents 0, 1, 2 or 3 substituents independently selected from
   linear or branched C₁-C₃-alkyl,
   linear or branched C₁-C₃-haloalkyl,
   linear or branched C₁-C₃-alkoxy,
   unsubstituted or substituted 6-membered aryl,
   unsubstituted or substituted 5- or 6-membered heteroaryl,
   unsubstituted or substituted bicyclic heteroaryl,
   unsubstituted or substituted 3- to 6-membered cycloalkyl,
   unsubstituted or substituted 5- or 6-membered heterocyclyl,
   unsubstituted or substituted 5- or 6-membered heterocyclylalkyl,
   unsubstituted or substituted 6-membered arylalkinyl, or
   unsubstituted or substituted 5- or 6-membered heteroarylalkinyl,
wherein a substituted aryl, heteroaryl, bicyclic heteroaryl, cycloalkyl, heterocyclyl, heterocyclylalkyl, arylalkinyl or heteroarylalkinyl group can optionally be substituted with 1, 2 or 3 substituents independently selected from
   halogen,
   C₁-C₃-alkyl,
   C₁-C₃-haloalkyl, and
   C₁-C₃-alkoxy;
R⁴ represents
   hydrogen
   unsubstituted or substituted linear or branched C₁-C₆-alkyl,
   a dialkylether group [R⁶(CH₂)ₓ—O—CH₂)_{y}—]
   with R⁶ representing a C₁-C₃-alkoxy group and
   with x and y independently representing an integer of 1, 2 or 3,
   unsubstituted or substituted 3- to 6-membered cycloalkyl,
   unsubstituted or substituted 5- or 6-membered heterocyclyl, or
   unsubstituted or substituted 6-membered aryl,
   unsubstituted or substituted 5- or 6-membered heteroaryl,
wherein alkyl, cycloalkyl, heterocyclyl, aryl and heteroaryl can optionally be substituted with 1 or 2 substituents, independently selected from
   halogen,
   C₁-C₃-alkoxy,
   C₆-cycloalkyloxy,
   carboxyl,
   aminocarbonyl,
   mono- or di-alkylaminocarbonyl,
   an amino group comprising —NH₂, mono- and dialkylamino,
   unsubstituted or substituted 3- to 6-membered cycloalkyl,
   unsubstituted or substituted 5- or 6-membered heterocyclyl,
   unsubstituted or substituted 6-membered aryl,
   unsubstituted or substituted 5- or 6-membered heteroaryl, and
   unsubstituted or substituted bicyclic heteroaryl,
wherein a substituted cycloalkyl, heterocyclyl, aryl, heteroaryl and bicyclic heteroaryl group can optionally be substituted with 1, 2 or 3 substituents independently selected from
   hydroxy,
   cyano,
   halogen,
   C₁-C₃-alkyl,
   C₁-C₃-haloalkyl,
   C₁-C₃-alkoxy,
   carboxyl,
   an amino (—NH₂) or mono- or di-alkylaminogroup,
   aminocarbonyl, and
   mono- or di-alkylaminocarbonyl,
wherein a monoalkylamino group and a monoalkylaminocarbonyl group may carry a further substituent on the mono-alkyl-chain, selected from
   C₁-C₃-alkoxy,
   unsubstituted or substituted 6-membered aryl, and
   unsubstituted or substituted 5- or 6-membered heteroaryl,
wherein a substituted aryl or heteroaryl group as a substituent of the mono-alkyl-chain can optionally be substituted with 1, 2 or 3 substituents independently selected from halogen, C₁-C₃-alkyl and C₁-C₃-haloalkyl;
with the proviso that in formulae (b-1) R⁴ does not represent hydrogen when R³ is absent or when R³ is methyl;
and in formulae (b-2) and (b-3) one of D1, D2 and D3 is present and represents
   a fused 6-membered aryl ring,
   a fused 5- or 6-membered heteroaryl ring,
   a fused 5- or 6-membered cycloalkyl ring, or
   a fused 5- or 6-membered heterocyclyl ring;
and the groups (b-2) and (b-3) carry 0, 1, 2 or 3 substituents, which are independently selected from
   halogen,
   linear or branched C₁-C₃-alkyl,
   linear or branched C₁-C₃-haloalkyl,
   linear or branched C₁-C₃-alkoxy;
and pharmaceutically acceptable salts thereof.

In one aspect of the invention, the compounds are characterized by having the selected central bicyclic heterocycle and by carrying in the benzimidazolyl-group of the terminal group B a nitrogen-bound substituent R⁴, i.e. forming a “N-substituted” cyclic group B.

In such a specifically preferred aspect, such “N-substituted” compounds are characterized in that in the formula (I-A) the substituent R⁴ represents:
unsubstituted or substituted linear or branched C₁-C₆-alkyl,
a dialkylether group [R⁶(CH₂)ₓ—O—CH₂)_{y}—]
with R⁶ representing a C₁-C₃-alkoxy group and
with x and y independently representing an integer of 1, 2 or 3,
unsubstituted or substituted 3- to 6-membered cycloalkyl,
unsubstituted or substituted 5- or 6-membered heterocyclyl, or
unsubstituted or substituted 6-membered aryl,
unsubstituted or substituted 5- or 6-membered heteroaryl,
   wherein alkyl, cycloalkyl, heterocyclyl, aryl and heteroaryl can optionally be substituted with 1 or 2 substituents, independently selected from
halogen,
C₁-C₃-alkoxy,
C₆-cycloalkyloxy,
carboxyl,
aminocarbonyl,
mono- or di-alkylaminocarbonyl,
an amino group comprising —NH₂, mono- and dialkylamino,
unsubstituted or substituted 3- to 6-membered cycloalkyl,
unsubstituted or substituted 5- or 6-membered heterocyclyl,
unsubstituted or substituted 6-membered aryl,
unsubstituted or substituted 5- or 6-membered heteroaryl, and
unsubstituted or substituted bicyclic heteroaryl,
wherein a substituted cycloalkyl, heterocyclyl, aryl, heteroaryl and bicyclic heteroaryl group can optionally be substituted with 1, 2 or 3 substituents independently selected from
   hydroxy,
   cyano,
   halogen,
   C₁-C₃-alkyl,
   C₁-C₃-haloalkyl,
   C₁-C₃-alkoxy,
   carboxyl,
   an amino (—NH₂) or mono- or di-alkylamino group,
   aminocarbonyl, and
   mono- or di-alkylaminocarbonyl,
wherein a monoalkylamino group and a monoalkylaminocarbonyl group may carry a further substituent on the mono-alkyl-chain, selected from
   C₁-C₃-alkoxy,
   unsubstituted or substituted 6-membered aryl, and
   unsubstituted or substituted 5- or 6-membered heteroaryl,
wherein a substituted aryl or heteroaryl group as a substituent of the mono-alkyl-chain can optionally be substituted with 1, 2 or 3 substituents independently selected from halogen, C₁-C₃-alkyl and C₁-C₃-haloalkyl. Very preferably, the substituent R⁴ is selected from the group of substituents shown in the respective position in the Examples below.

In a further aspect, the compounds of the invention are characterized by having the selected central bicyclic heterocycle and the group B contains at least 3 (three) rings. Therein, the group B may represent a ring system of at least 3 fused rings, wherein the individual fused rings may have a structure as defined herein. Embodiments having a group B with at least three rings include compounds wherein the group B is represented by a group (b-1), carrying a substituent R³ with a cyclic structure, i.e. R³ does not represent hydrogen, but a (substituted or unsubstituted) aryl, heteroaryl, cycloalkyl or heterocyclyl ring, or a fused bicyclic ring system, including a phenyl ring carrying a fused cycloalkyl or heterocyclyl ring as defined herein, which may be bound via a direct bond or a C₁-C₃-alkyl-chain or an alkinyl-chain as defined herein.

This means, that the expression “the group B contains at least 3 rings” is not limited to fused tricyclic ring systems.

Accordingly, the compounds of the present invention can further be characterized by the formula (I-A) as defined above, wherein in the definition of the group according to formula (b-1) R⁴ does not represent hydrogen (but a N-bound substituent) when either R³ is absent or when R³ has the meaning as defined above, including R³ is no substituent with cyclic structure but a “linear substituent”, which means selected from linear or branched C₁-C₃-alkyl, linear or branched C₁-C₃-haloalkyl, or linear or branched C₁-C₃-alkoxy, as defined herein.

In a preferred aspect the compounds of the invention are characterized by the formula (I-A) as defined above, wherein in the definition of R⁴ is not hydrogen, i.e. hydrogen is excluded. That means, that very preferably the substituent R⁴ is a group as defined in any of the embodiments described anywhere herein excluding hydrogen.

In a further preferred aspect, the compounds of the invention are characterized by the formula (I-A) as defined above, wherein in the definition of the group according to formula (b-1) R⁴ does not represent hydrogen (but a N-bound substituent) and R³ is selected from substituents with a cyclic structure as defined above.

A further aspect of the invention relates to the compounds of the formula (I-A) as defined herein, which are represented by the following formula (I-B)

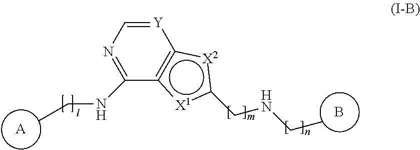

wherein
I is an integer of 1 or 2; and
m and n are independently an integer of 1, 2 or 3.

### Definitions

The term “substituted” means that one or more hydrogen atoms on the designated atom or group are replaced with a selection from the indicated group, provided that the designated atom's normal valency under the existing circumstances is not exceeded.

The term “optionally substituted”, “optional substituent(s)” or “possible substituent(s)” means that the number of substituents can be equal to or different from zero. Unless otherwise indicated, it is possible that optionally substituted groups are substituted with as many optional substituents as can be accommodated by replacing a hydrogen atom with a non-hydrogen substituent on any available carbon or nitrogen atom. Commonly, it is possible for the number of optional substituents, when present, to be 1, 2, 3, 4 or 5, in particular 1, 2 or 3.

If used herein, the term “one or more”, e.g. in the definition of the substituents of the compounds of general formula (I-A) and (I-B) of the present invention, means “1, 2, 3, 4 or 5, particularly 1, 2, 3 or 4, more particularly 1, 2 or 3, even more particularly 1 or 2”.

The term “comprising” or “containing” when used in the claims or specification includes “consisting of”.

If within the present specification any item is referred to as “as mentioned herein” or “as defined (anywhere) herein”, it means that it may be mentioned anywhere in the present specification or may have the meaning as defined anywhere in the present specification.

The terms used in the claims and specification have the following meanings:

“Halogen” or “halogen atom” means a fluorine, chlorine, bromine or iodine atom, particularly a fluorine, chlorine or bromine atom, a preferred selection relates to chlorine or fluorine, a further preferred selection relates to bromine or fluorine, most preferred is fluorine.

The term “C₁-C₆-alkyl” means a linear or branched, saturated, monovalent hydrocarbon group having 1, 2, 3, 4, 5 or 6 carbon atoms, e.g. a methyl, ethyl, n-propyl, isopropyl, n-butyl, iso-butyl, pentyl or hexyl group. Methyl, ethyl, n-propyl, iso-propyl, n-butyl and iso-butyl groups are preferred. More preferred is a linear or branched “C₁-C₃-alkyl” group, such as in particular methyl, ethyl, n-propyl and iso-propyl. Preferred unsubstituted alkyl-groups are selected from methyl and ethyl.

The C₁-C₆-alkyl or the C₁-C₆-alkyl group may optionally be substituted with 1 or 2 substituents, preferably with 1 substituent. In such a case the substituted alkyl group is preferably a substituted C₁-C₃-alkyl group (i.e. a substituted C₁-, C₂- or C₃-alkyl group), more preferably a substituted methyl or ethyl group. Such optional substituents are preferably selected from the group consisting of: halogen (forming a halogen-substituted C₁-C₃-alkyl group as defined below and herein also indicated as “C₁-C₃-haloalkyl”), such as preferably difluoroalkyl or trifluoroalkyl, C₁-C₃-alkoxy such as preferably methoxy, a cycloalkyloxy group, such as preferably a C₆-cycloalkyloxy group (cyclohexyloxy), a carboxyl group [—(C═O)OH], an aminocarbonyl group [NH₂(C═O)—], a mono- or dialkylaminocarbonyl group such as preferably a methylaminocarbonyl group [CH₃NH(C═O)—] or a dimethylaminocarbonyl group [(CH₃)₂N(C═O)—], an amino group comprising —NH₂, mono- and di-alkylamino, such as preferably mono- or di-methylamino, 3- to 6-membered cycloalkyl (also designated as C₃-C₆-cycloalkyl) containing 3, 4, 5 or 6 carbon atoms, such as preferably cyclopropyl and cyclohexyl, unsubstituted or substituted 5- or 6-membered heterocyclyl, such as preferably a substituted 6-membered heterocyclyl, unsubstituted or substituted 6-membered aryl (phenyl), such as preferably an unsubstituted 6-membered aryl (phenyl), unsubstituted or substituted 5- or 6-membered heteroaryl, such as preferably an unsubstituted 6-membered heteroaryl, and unsubstituted or substituted bicyclic heteroaryl such as preferably a benzimidazolyl group.

A substituted heterocyclyl, aryl, heteroaryl and bicyclic heteroaryl group as a substituent of alkyl may also carry 1, 2 or 3 substituents independently selected from hydroxy, cyano, halogen, C₁-C₃-alkyl as defined herein such as preferably methyl, C₁-C₃-haloalkyl as defined herein such as preferably difluoroethyl or trifluoromethyl (CF₃), C₁-C₃-alkoxy as defined herein such as preferably methoxy, a carboxyl group, an amino (—NH₂) or mono- or di-alkylamino group, an aminocarbonyl group as defined herein, and a mono- or di-alkylaminocarbonyl group, wherein a mono-alkylaminocarbonyl group may carry a further substituent on the mono-alkyl-chain, selected from C₁-C₃-alkoxy, unsubstituted or substituted 6-membered aryl, and unsubstituted or substituted 5- or 6-membered heteroaryl, wherein a substituted aryl or heteroaryl group as a substituent of the mono-alkyl-chain can carry 1, 2 or 3 substituents independently selected from halogen, C₁-C₃-alkyl and C₁-C₃-haloalkyl, preferably a further substituent on the mono-alkyl-chain of a mono-alkylaminocarbonyl group is selected from a halogen-substituted 5- or 6-membered heteroaryl (more preferably a fluoro-pyridinyl group).

The term “dialkylether” or “dialkylether group” as used herein means a C₃-C₇-alkyl group as defined above, wherein one CH₂-group in the alkyl-chain is replaced by —O—, resulting in a group [—(CH₂)ₓ—O—CH₂)_{y}—] with x and y independently representing an integer of 1, 2 or 3. Such a dialkylether group as a substituent R⁴ carries a further substituent R⁶, resulting in a group [R⁶(CH₂)ₓ—O—CH₂)_{y}—]. Therein, R⁶ represents a substituent selected from the group of C₁-C₃-alkoxy. If R⁶ represents a hydrogen atom the dialkylether group is unsubstituted and corresponds to a group “alkoxy” as defined herein separately. Preferred substituents R⁶ are selected from a C₁-C₃-alkoxy group, such as in particular methoxy and ethoxy.

The term “C₁-C₃-haloalkyl” means a linear or branched, saturated, monovalent C₁-C₃-alkyl group, having the meaning as defined above, in which one or more of the hydrogen atoms are replaced, identically or differently, with a halogen atom. Particularly, said halogen atom is a chlorine or fluorine atom. More particularly, said halogen atom is a fluorine atom and even more particularly, all said halogen atoms are fluorine atoms (“C₁-C₃-fluoroalkyl”). Said C₁-C₃-haloalkyl group is, for example, fluoromethyl, difluoromethyl, trifluoromethyl, 2-fluoroethyl, 2,2-difluoroethyl, 2,2,2-trifluoroethyl, pentafluoroethyl, 3,3,3-trifluoropropyl or 1,3-difluoropropan-2-yl, wherein a trifluoromethyl-group (CF₃) is particularly preferred.

The term “C₁-C₃-alkoxy” means a linear or branched, saturated, monovalent group of formula (C₁-C₃-alkyl)-O—, in which the term “C₁-C₃-alkyl” is as defined supra, e.g. a methoxy, ethoxy, n-propoxy or isopropoxy group, with a methoxy-group and an iso-propoxy group being particularly preferred.

The term cycloalkyloxy relates to a cycloalkyl-O— group, with a cycloalkyl group as defined below being bound via an oxygen (—O—). Cycloalkyloxy includes “C₃-C₆-cycloalkyloxy”, with C₆-cycloalkyloxy (cyclohexyloxy) being preferred.

A “carboxyl group” indicates a group [—(C═O)OH].

The term “mono- or di-alkylamino” indicates an amino group (—NH₂), wherein one or both hydrogens are replaced by the same of different C₁-C₃-alkyl groups. Preferred are mono- and dimethylamino groups, more preferred are dimethylamino groups, in particular as a substituent of “substituted C₁-C₃-alkyl” on the position of R⁴.

The term “aminocarbonyl group” indicates a group [NH₂—(C═O)—].

The term “mono- or dialkylaminocarbonyl group” indicates an aminocarbonyl group [NH₂—(C═O)—], wherein one or both hydrogen are replaced by a C₁-C₃-alkyl group. A preferred mono-alkylaminocarbonyl group is a methylaminocarbonyl group [CH₃NH(C═O)—]. A preferred di-alkylaminocarbonyl group is a dimethylaminocarbonyl group [(CH₃)₂N(C═O)—].

Generally, the term “aryl” includes aromatic hydrocarbon residues containing 6 to 14 carbon atoms (excluding the carbon atoms of the possible substituents), which may be monocyclic or bicyclic, including, for example: phenyl, naphthyl, phenanthrenyl and anthracenyl. Preferred is 6-membered aryl, such as phenyl.

Generally, the term “heteroaryl” includes heteroaromatic hydrocarbon residues containing 4 to 9 ring carbon atoms, which additionally contain 1 to 3 of the same or different heteroatoms selected from S, O and N in the ring, and therefore form 5- to 12-membered heteroaromatic residues which may be monocyclic or bicyclic.

Monocyclic heteroaryl groups preferably include 5- and 6-membered monocyclic heteroaryl groups, such as pyridyl (pyridinyl), pyridyl-N-oxide, pyridazinyl, pyrimidyl, pyrazinyl, thienyl (thiophenyl), furyl, pyrrolyl, pyrazolyl, imidazolyl, triazolyl, tetrazolyl, thiazolyl, isothiazolyl, oxazolyl or isoxazolyl, including from the group of 5-membered heteroaryl, for example thiazolyl such as thiazol-2-yl, 2-thiazol-2-yl, 2-thiazol-4-yl, thienyl (thiophenyl), such as thien-3-yl, pyrazolyl such as 1-pyrazol-4-yl, 3-pyrazol-5-yl, imidazolyl such as imidazole-2-yl, 2-imidazol-4-yl, 1-imidazol-4-yl, triazolyl such as 1-triazol-3-yl, 1-triazol-4-yl, such as 1,2,4-triazol-3-yl or 1,2,3-triazol-4-yl, oxazolyl such as 2-oxazol-4-yl, 2-oxazol-5-yl, iso-oxazolyl such as iso-oxazol-4-yl, oxadiazolyl such as 1,2,4-oxadiazol-3-yl, tetrazolyl and from the group of 6-membered heteroaryl, for example, pyridyl (pyridinyl) such as pyrid-1-yl, pyrid-2-yl, pyrid-3-yl, pyrid-4-yl, 2-pyrid-4-yl, 2-pyrid-6-yl, 3-pyrid-5-yl (pyridin-1-yl, pyridin-2-yl, pyridin-3-yl, pyridin-4-yl, 2-pyridin-4-yl, 2-pyridin-6-yl, 3-pyridin-5-yl, pyrimidin-2-yl, pyrimidin-4-yl, pyrimidin-5-yl. Preferred heteroaryl groups are pyridinyl, pyrimidinyl, imidazolyl, oxazolyl, iso-oxazolyl and tetrazolyl.

Bicyclic heteroaryl groups preferably include indolizinyl, indolyl, benzo[b]thienyl, benzo[b]furyl, indazolyl, quinolyl, isoquinolyl, naphthyridinyl, quinazolinyl, quinoxalinyl, and benzimidazolyl such as benzimidazol-2-yl, benzimidazol-4-yl, benzimidazol-5-yl. A benzimidazolyl group is particularly preferred.

Generally, the term “cycloalkyl” includes aliphatic rings containing 3 to 8, more preferably 3 to 6 ring carbon atoms. Cycloalkyl includes a cyclopropyl group, a cyclobutyl group, a cyclopentyl group, a cyclohexyl group, a cycloheptyl group and a cyclooctyl group, with a cyclopropyl group and a cyclohexyl group being preferred.

Generally the term “heterocyclyl” includes saturated or unsaturated mono- or bicyclic 4- to 8-membered heterocyclic residues containing 1 to 3, preferably 1 to 2 same or different hetero atoms selected from N, O and S, including azetidinyl, oxetanyl, pyrrolidinyl, pyrazolidinyl, imidazolidinyl, tetrahydrofuranyl, dioxolanyl, tetrahydrothiophenyl, oxathiolanyl, piperidinyl, piperazinyl, tetrahydropyranyl, thianyl, dithianyl, trithianyl, tetrahydrothiopyranyl, morpholinyl, thiomorpholynyl, dioxanyl, etc., such as azetidin-1-yl, azetidin-2-yl, azetidin-3-yl, tetrahydrofuran-2-yl, tetrahydrofuran-3-yl, tetrahydro-thiophen-2-yl, tetrahydro-thiophen-3-yl, pyrrolidin-1-yl, pyrrolidin-2-yl, pyrrolidin-3-yl, morpholin-1-yl, morpholin-2-yl, morpholin-3-yl, piperidin-1-yl, piperidin-2-yl, piperidin-3-yl, piperidin-4-yl, piperazin-1-yl, piperazin-2-yl, tetrahydropyran-2-yl, tetrahydropyran-3-yl, tetrahydropyran-4-yl, etc. Particularly preferred are 5- or 6-membered heterocyclyl groups, such as pyrrolidinyl, dioxolanyl, dioxanyl, piperidinyl, piperazinyl and morpholinyl residues, with a morpholinyl group and a piperazinyl group being particularly preferred.

The aryl, heteroaryl, bicyclic heteroaryl, cycloalkyl or heterocyclyl groups can be bound via a direct bond, a C₁-C₃-alkyl-chain, preferably a direct bond or a C₁- or C₂-alkyl-chain or an alkinyl-chain, such as preferably an ethinyl-chain (—C≡C—), or the aryl, heteroaryl, cycloalkyl or heterocyclyl groups can be condensed with aromatic rings forming fused ring systems as defined herein.

The aryl, heteroaryl, bicyclic heteroaryl, cycloalkyl or heterocyclyl group, including fused aryl, heteroaryl, bicyclic heteroaryl, cycloalkyl and heterocyclyl groups, may carry 1, 2 or 3 of the same or different substituents, independently selected from halogen as defined above such as preferably F, Br and Cl, C₁-C₃-alkyl such as preferably methyl or ethyl, C₁-C₃-haloalkyl as defined above such as preferably trifluoromethyl, C₁-C₃-alkoxy as defined above such as preferably methoxy and C₆-cycloalkyloxy. Particularly preferred is a methoxy-phenyl group and a chloro-phenyl group, in particular as a substituent of “substituted C₁-C₃-alkyl” on the position of R⁴. Also preferred is a pyridinyl-group or a methylpiperazinyl-group as a substituent of “substituted C₁-C₃-alkyl” on the position of R⁴.

The aryl, heteroaryl, bicyclic heteroaryl, cycloalkyl or heterocyclyl group may also carry 1, 2 or 3 substituents as defined above in context with the possible substituents of alkyl supra.

The aryl, heteroaryl, bicyclic heteroaryl, cycloalkyl or heterocyclyl groups as defined herein may form one of the groups A and/or B as defined herein.

In the formula (I-A) “I” represents an integer of 1 or 2, preferably I=1.

In the formula (I-A) “L¹” and “L²” each represent a linker group or a so-called spacer, i.e. an “alkyl-spacer”, which comprises 1 to 7 carbon atoms. Such an alkyl-spacer group or linker “L¹” and “L²” is independently selected from
a linear C₁-C₃-alkyl group —[CH₂]ₘ or —[CH₂]ₙ—, respectively, wherein m and n are independently an integer of 1, 2 or 3; or
a branched C₁-C₄-alkyl group, such as preferably a 2-dimethylethyl group

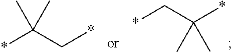

or
a C₃-C₆-cycloalkyl group, which forms a ring together with the nitrogen atom to which it is bonded, such as preferably for “L¹”

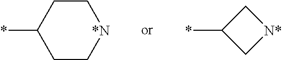

and
preferably for “L²”

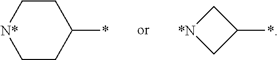

A 4-membered cycloalkyl group, which forms a ring together with the nitrogen atom to which it is bonded, is most preferred.

If the linker “L¹” and/or “L²” has the meaning of a linear C₁-C₃-alkyl group, the linker can be replaced in formula (I-A) by —[CH₂]ₘ and/or —[CH₂]ₙ— as defined herein. If both linker “L¹” and “L²” are linear C₁-C₃-alkyl, the compounds can be represented by the formula (I-B)

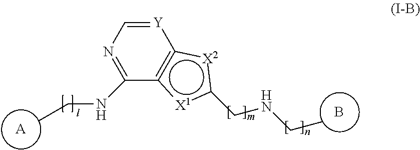

wherein
I is an integer of 1 or 2; and
m and n are independently an integer of 1, 2 or 3; and
all other substituents may have the meaning as defined anywhere herein.

In the formula (I-B) “m” and “n” independently represent an integer of 1, 2 or 3, preferably m=2 and preferably n=1 or 2, even more preferably m and n both represent 2.

In one aspect of the invention the linker “L²” has the meaning of a linker group comprising 1 to 7 carbon atoms selected from
a linear C₁-C₃-alkyl group —[CH₂]ₙ—, wherein n represents an integer of 1, 2 or 3,
a branched C₁-C₄-alkyl group, and
a C₃-C₆-cycloalkyl group, which forms a ring together with the nitrogen atom to which it is bonded, as defined herein.

In the formula (I-A) and/or (I-B) X¹ and X² are selected from:
X¹=N, S or O; and
X²=N, S or O;
   with the proviso that one of X¹ and X² is N.

In the formula (I-A) and/or (I-B)
Y represents N or CR⁵.

Therein, R⁵ represents an optional substituent in case of Y=CR⁵, and R⁵ is preferably selected from halogen, linear or branched C₁-C₃-alkyl and linear or branched C₁-C₃-haloalkyl, each as defined above. If no substituent is present, then R⁵ represents hydrogen.

In a further aspect, the compounds of the invention are represented by one of the following formulae (I-C), (I-D), (I-E), (I-F) or (I-G):

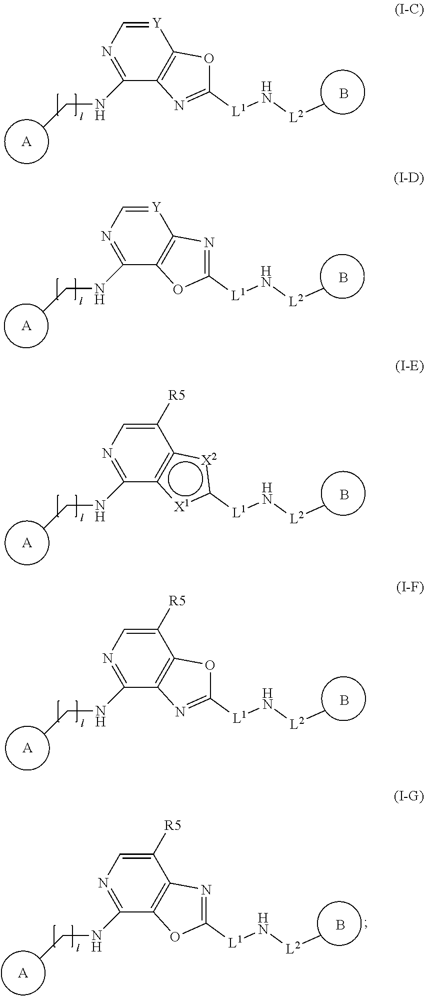

wherein the remaining substituents have the meaning as defined anywhere herein.

Generally, the group A represents a group (a-1)

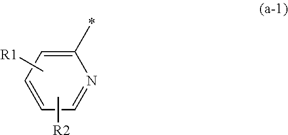

The group (a-1) is a pyridinyl-group, which carries 0 substituents (R¹/R² represent hydrogen) or 1 or 2 same or different substituents R¹/R², independently selected from halogen as defined above such as preferably F, Br and Cl, C₁-C₃-alkyl such as preferably methyl, C₁-C₃-haloalkyl as defined above such as preferably trifluoromethyl, and C₁-C₃-alkoxy as defined above such as preferably methoxy.

From the group (a-1) the following groups are preferred:

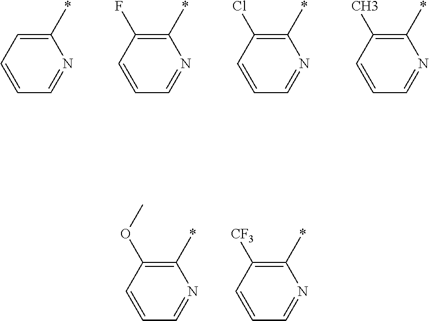

Therefrom a group (a-1) with the following structures are more preferred:

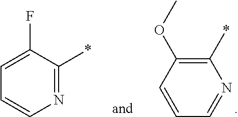

Therefrom a group (a-1) with the following structure is most preferred:

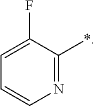

Generally, the group B represents one of the following groups (b-1), (b-2) and (b-3)

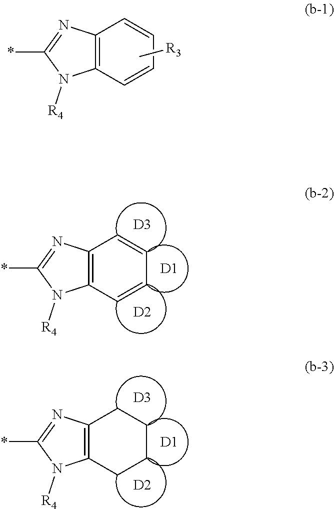

Therein, a group (b-1) represents a benzimidazolyl group, which carries 0 substituents R³ (i.e. R³ represents hydrogen) or 1, 2 or 3, preferably 1 or 2, same or different substituents R³ as defined herein. If present, then R³ preferably represents 1 substituent as defined anywhere herein.

As mentioned above, a substituent R³ can be selected from so-called “linear substituents”, including linear or branched C₁-C₃-alkyl, linear or branched C₁-C₃-haloalkyl and linear or branched C₁-C₃-alkoxy, or R³ can be selected from so-called “cyclic substituents”, including an aryl, heteroaryl, bicyclic heteroaryl, cycloalkyl and heterocyclyl group as defined above, which is bound via a direct bond or which is bound via a C₁-C₃-alkyl-chain, preferably a C₁-alkyl-chain, or an alkinyl-chain, such as preferably an ethinyl-chain.

R³ is not absent but a substituent as defined herein, excluding methyl, when R⁴ is hydrogen. Preferably, R³ is selected from the above defined group of “cyclic substituents” when R⁴ is hydrogen.

Preferably, R³ is selected from unsubstituted or substituted phenyl, unsubstituted or substituted 5- or 6-membered heteroaryl, unsubstituted or substituted bicyclic heteroaryl, unsubstituted or substituted 3- to 6-membered cycloalkyl, unsubstituted or substituted 5- or 6-membered heterocyclyl, unsubstituted or substituted 5- or 6-membered heterocyclylalkyl, unsubstituted or substituted 6-membered arylalkinyl or unsubstituted or substituted 5- or 6-membered heteroarylalkinyl, wherein a substituted aryl, heteroaryl and bicyclic heteroaryl group can carry 1, 2 or 3 substituents independently selected from halogen, C₁-C₃-alkyl, C₁-C₃-haloalkyl, and C₁-C₃-alkoxy.

An aryl, heteroaryl, bicyclic heteroaryl, cycloalkyl or heterocyclyl ring bound via a C₁-C₃-alkyl-chain, preferably a C₁-alkyl-chain, corresponds to R³ representing an arylalkyl, a heteroarylalkyl, a cycloalkylalkyl or a heterocyclylalkyl group, wherein “alkyl” preferably represents C₁-C₃-alkyl. A heterocyclylalkyl group, such as a piperazinylmethyl group or a morpholinylmethyl group is preferred.

An aryl, heteroaryl, bicyclic heteroaryl, cycloalkyl or heterocyclyl ring bound via an alkinyl-chain, such as preferably an ethinyl-chain, corresponds to R³ representing an arylalkinyl, a heteroarylalkinyl, a cycloalkylalkinyl or a heterocyclylalkinyl group, wherein “alkinyl” preferably represents ethinyl. An arylalkinyl group, such as a phenylethinyl group, and a heteroarylalkinyl group, such as a pyridinylethinyl group, are preferred. Therein the aryl, heteroaryl, bicyclic heteroaryl, cycloalkyl or heterocyclyl ring may carry 1, 2 or 3 of the same or different substituents selected from those defined herein for the respective groups, preferably selected from halogen as defined above such as preferably F, Br and Cl, C₁-C₃-alkyl such as preferably methyl, C₁-C₃-haloalkyl as defined above such as preferably trifluoromethyl, and C₁-C₃-alkoxy as defined above such as preferably methoxy. Therewith, such groups are also designated herein as “unsubstituted or substituted arylalkinyl”, “unsubstituted or substituted heteroarylalkinyl”, “unsubstituted or substituted cycloalkylalkinyl” and “unsubstituted or substituted heterocyclylalkinyl”.

As explained above, a group (b-1) preferably carries a cyclic substituent R³ resulting in a group B which contains at least 3 (three) rings, Examples of possible groups (b-1) with a cyclic substituent comprise the following:

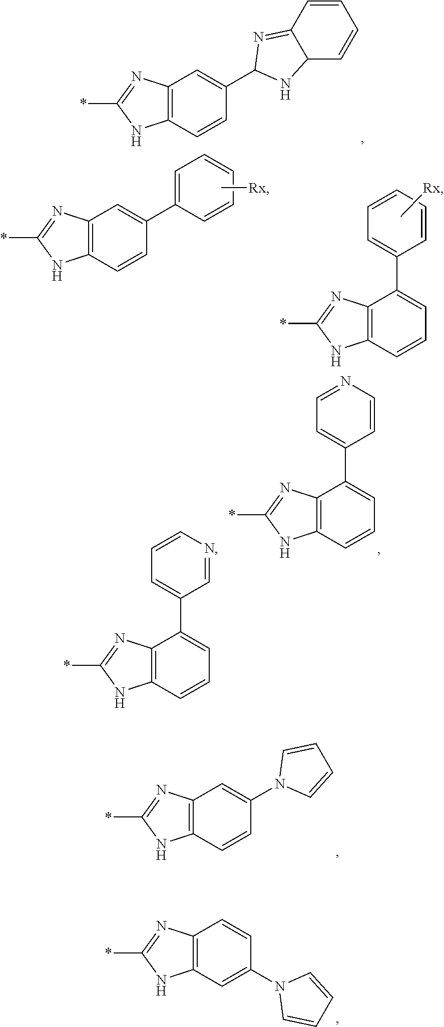

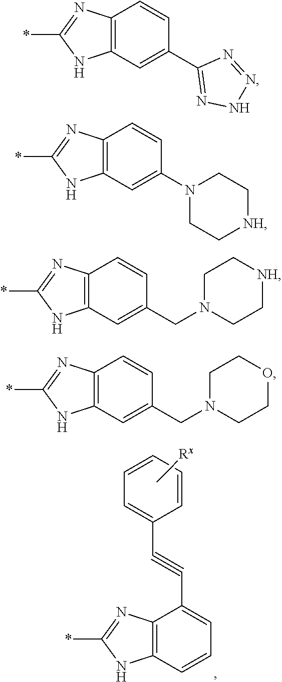

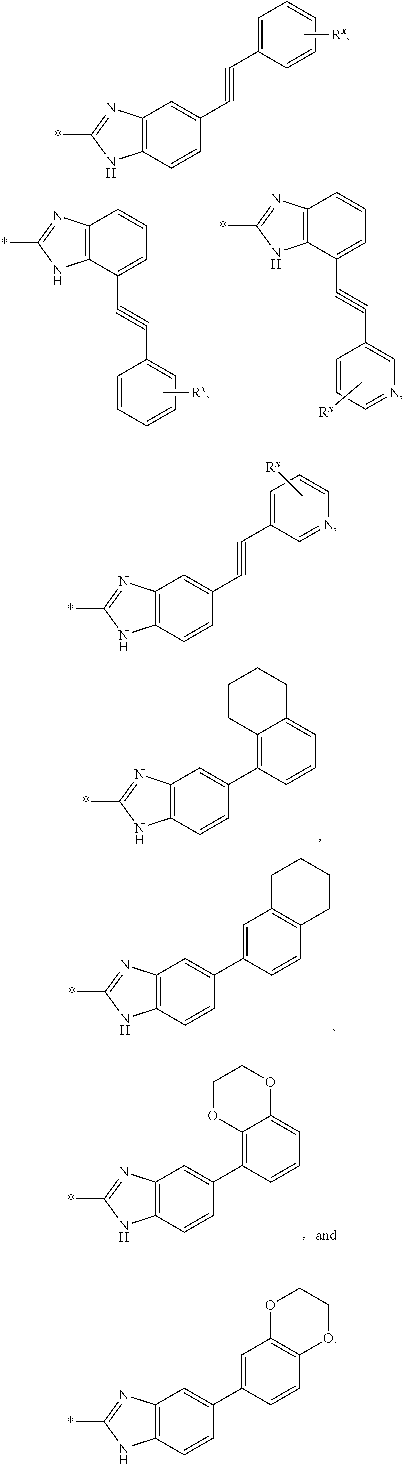

It is generally possible that an unsubstituted or substituted aryl ring is bound directly, corresponding to R³ or R⁴ representing “unsubstituted or substituted aryl”. Regarding possible substituents reference is made to the definition above. In particular for an aryl group directly bound to the group B, e.g. as the substituent R⁴, preferred substituents are selected from halogen, more preferably Cl, and C₁-C₃-alkoxy, more preferably methoxy.

The groups (b-2) and (b-3) represent fused (condensed) ring systems, wherein in formulae (b-2) and (b-3) a fused aryl, heteroaryl, cycloalkyl or heterocyclyl ring as defined above is present in one of the positions indicated by D1, D2 and D3, preferably forming a fused tricyclic ring system.

The groups (b-2) and (b-3) may optionally carry 1, 2 or 3 same or different substituents, which are independently selected from halogen, linear or branched C₁-C₃-alkyl, linear or branched C₁-C₃-haloalkyl and linear or branched C₁-C₃-alkoxy, each as defined above. Such optional substituent is hereinafter also represented by R^{x}. Such substituent(s) R^{x} on the group (b-2) or (b-3) refer to one or more additional substituents, i.e. further to the N-bound R⁴ substituent.

Examples of possible groups (b-2) with a fused tricyclic structure comprise the following:

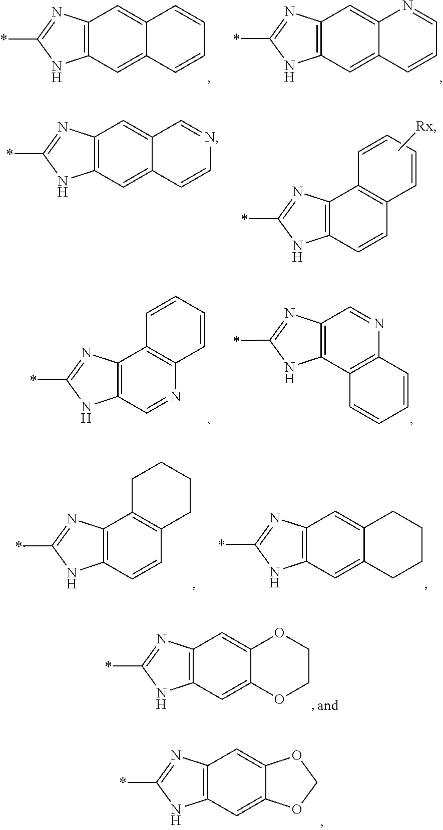

especially preferred is

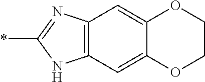 [SMILES: *c1nc2cc3c(cc2[nH]1)OCCO3]

Examples of possible groups (b-3) with a fused tricyclic structure comprise the following:

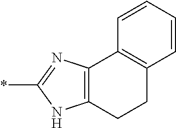 [SMILES: *c1nc2c([nH]1)CCc1ccccc1-2]

The compounds of the formula (I-A) and (I-B) of the present invention are characterized by comprising a substituent R⁴ in the group B, forming a “N-substituted” cyclic group B. In the embodiments of the invention, wherein R⁴ is not directly bound substituted or unsubstituted aryl, the “N-substituted” cyclic group B can be designated as “N-alkylated” (cyclic) group B. Therein, the term “N-alkylated” is understood to include a substitution with an alkyl group, a dialkylether group as well as cycloalkyl or a heterocyclyl-group as defined herein.

In such cases, particularly, the R⁴ substituent represents a linear or branched C₁-C₆-alkyl group, a dialkylether group [R⁶(CH₂)ₓ—O—CH₂)_{y}—] as defined above, a 3- to 6-membered cycloalkyl, or 5- or 6-membered heterocyclyl, which can be substituted or unsubstituted.

In the case of R⁴ representing a substituted alkyl group, reference is made to possible alkyl-substituents as defined above, wherein especially preferred alkyl substituents include alkoxy, unsubstituted cycloalkyl, unsubstituted or substituted 6-membered heterocyclyl, unsubstituted or substituted 6-membered aryl, unsubstituted heteroaryl and dialkylamino groups.

In the case of R⁴ representing a substituted dialkylether group, reference is made to the definition of “dialkylether” and its possible substituents R⁶ as defined above, wherein especially preferred dialkylether substituents include alkoxy groups.

In the case of R⁴ representing a substituted cycloalkyl or heterocyclyl group, reference is made to the definition of possible substituents of such groups anywhere herein, wherein especially preferred substituents include C₁-C₃-alkyl.

In the case of R⁴ representing a substituted aryl group, reference is made to possible aryl- (phenyl-) substituents as defined above, wherein especially preferred substituents include alkoxy or halogen groups.

Preferably, the R⁴ substituent aryl, alkyl, dialkylether, cycloalkyl and heterocyclyl can be substituted with 1 or 2 substituents, in particular such substituents as defined above for phenyl, for C₁-C₃-alkyl and for dialkylether.

In one aspect, the compounds (I-A) and (I-B) of the invention comprise a group (b-1) as defined herein carrying a substituent R⁴ which is not hydrogen but a group as defined herein for R⁴.

In a further aspect, the compounds (I-A) and (I-B) of the invention comprise a group (b-2).

In such embodiments, the following groups (b-2) are preferred:

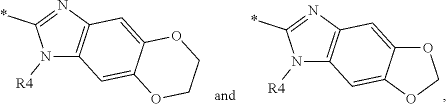

among which the following group is even more preferred:

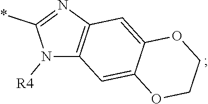

and
wherein R⁴ has the meaning as defined anywhere herein (including or excluding hydrogen).

Examples of such groups (b-2) comprise:

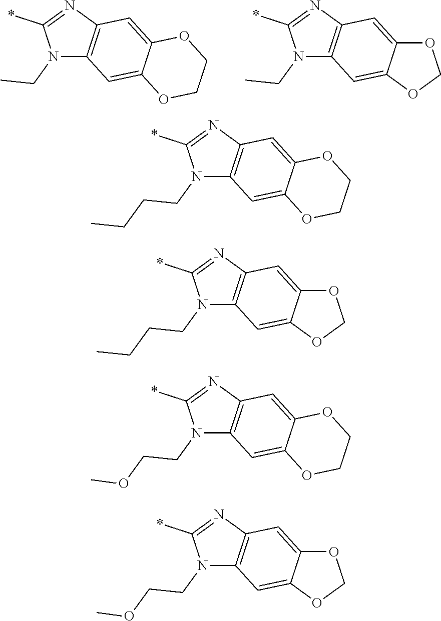

Among these Examples of group (b-2) those with a fused dioxane ring are preferred.

If used anywhere herein, e.g. in the formulae (a-1), (b-1), (b-2) and (b-3), “*” indicates the binding position.

A further aspect relates to compounds of the formula (I-A) and (I-B) as defined above, wherein
A represents a group (a-1)

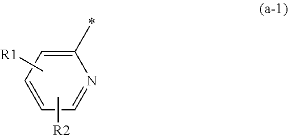

wherein * indicates the binding position;
R¹ and R² independently represent
   hydrogen,
   halogen,
   linear or branched C₁-C₃-alkyl,
   linear or branched C₁-C₃-haloalkyl, or
   linear or branched C₁-C₃-alkoxy;
B represents one of the following groups (b-1), (b-2) and (b-3)

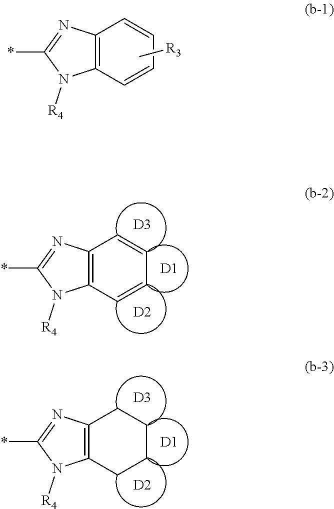

wherein * indicates the binding position;
R³ represents 0, 1, 2 or 3 substituents independently selected from
   linear or branched C₁-C₃-alkyl,
   linear or branched C₁-C₃-haloalkyl,
   linear or branched C₁-C₃-alkoxy,
   unsubstituted or substituted 6-membered aryl,
   unsubstituted or substituted 5- or 6-membered heteroaryl,
   unsubstituted or substituted bicyclic heteroaryl,
   3- to 6-membered cycloalkyl,
   5- or 6-membered heterocyclyl,
   5- or 6-membered heterocyclylalkyl, or
   6-membered arylalkinyl
wherein a substituted aryl, heteroaryl and bicyclic heteroaryl group can optionally be substituted with 1, 2 or 3 substituents independently selected from
   halogen,
   C₁-C₃-alkyl,
   C₁-C₃-haloalkyl, and
   C₁-C₃-alkoxy;
R⁴ represents
   linear or branched C₁-C₆-alkyl,
   a dialkylether group [R⁶(CH₂)ₓ—O—CH₂)_{y}—]
   with R⁶ representing a C₁-C₃-alkoxy group and
   with x and y independently representing an integer of 1, 2 or 3,
   3- to 6-membered cycloalkyl, or
   5- or 6-membered heterocyclyl,
   6-membered aryl,
wherein alkyl, cycloalkyl, heterocyclyl and aryl can optionally be substituted with 1 or 2 substituents, independently selected from
   C₁-C₃-alkoxy,
   carboxyl,
   aminocarbonyl,
   mono- or di-alkylaminocarbonyl,
   an amino (—NH₂) or mono- or di-alkylamino group
   3- to 6-membered cycloalkyl, and
   5- or 6-membered heterocyclyl,
   unsubstituted or substituted 6-membered aryl,
   unsubstituted or substituted 5- or 6-membered heteroaryl, and
   unsubstituted or substituted bicyclic heteroaryl,
wherein a substituted aryl, heteroaryl and bicyclic heteroaryl group can optionally be substituted with 1, 2 or 3 substituents independently selected from
   hydroxy,
   cyano,
   halogen,
   C₁-C₃-alkyl,
   C₁-C₃-haloalkyl,
   C₁-C₃-alkoxy,
   carboxyl,
   an amino (—NH₂) or mono- or di-alkylaminogroup,
   aminocarbonyl, and
   mono- or di-alkylaminocarbonyl,
wherein a monoalkylamino group and a monoalkylaminocarbonyl group may carry a further substituent on the mono-alkyl-chain, selected from
   C₁-C₃-alkoxy,
   unsubstituted or substituted 6-membered aryl, and
   unsubstituted or substituted 5- or 6-membered heteroaryl,
wherein a substituted aryl or heteroaryl group as a substituent of the mono-alkyl-chain can optionally be substituted with 1, 2 or 3 substituents independently selected from halogen, C₁-C₃-alkyl and C₁-C₃-haloalkyl;
and in formulae (b-2) and (b-3) one of D1, D2 and D3 is present and represents
   a fused 6-membered aryl ring,
   a fused 5- or 6-membered heteroaryl ring,
   a fused 5- or 6-membered cycloalkyl ring, or
   a fused 5- or 6-membered heterocyclyl ring;
and the groups (b-2) and (b-3) carry 0, 1, 2 or 3 substituents, which are independently selected from
   halogen,
   linear or branched C₁-C₃-alkyl,
   linear or branched C₁-C₃-haloalkyl,
   linear or branched C₁-C₃-alkoxy.

A further aspect relates to compounds of the formula (I-A) and (I-B) as defined above, wherein
A represents a group (a-1)

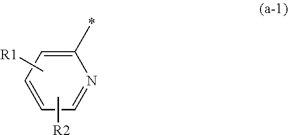

wherein * indicates the binding position;
R¹ and R² independently represent
   hydrogen,
   halogen,
   linear or branched C₁-C₃-alkyl,
   linear or branched C₁-C₃-haloalkyl, or
   linear or branched C₁-C₃-alkoxy;
B represents one of the following groups (b-1), (b-2) and (b-3)

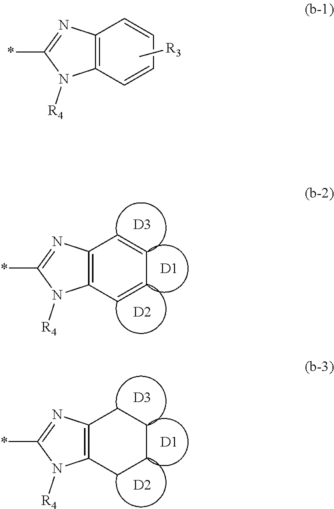

wherein * indicates the binding position;
R³ represents 0, 1, 2 or 3 substituents independently selected from
   linear or branched C₁-C₃-alkyl,
   linear or branched C₁-C₃-haloalkyl,
   linear or branched C₁-C₃-alkoxy,
   unsubstituted or substituted phenyl,
   unsubstituted or substituted 5- or 6-membered heteroaryl,
   unsubstituted or substituted bicyclic heteroaryl,
   6-membered heterocyclyl,
   6-membered heterocyclylalkyl,
   phenylethinyl, or
   pyridinylethinyl,
wherein a substituted phenyl, heteroaryl and bicyclic heteroaryl group can optionally be substituted with 1, 2 or 3 substituents independently selected from
   halogen,
   C₁-C₃-alkyl,
   C₁-C₃-haloalkyl, and
   C₁-C₃-alkoxy;
R⁴ represents
   linear or branched C₁-C₆-alkyl,
   a dialkylether group [R⁶(CH₂)ₓ—O—CH₂)_{y}—]
   with R⁶ representing a C₁-C₃-alkoxy group and
   with x and y independently representing an integer of 1, 2 or 3,
   5- or 6-membered unsubstituted heterocyclyl, or
   substituted or unsubstituted phenyl,
wherein substituents of phenyl are selected from
   halogen, and
   C₁-C₃-alkoxy; and
wherein alkyl can optionally be substituted with 1 or 2 substituents, independently selected from
   halogen,
   C₁-C₃-alkoxy,
   C₆-cycloalkyloxy,
   carboxyl,
   aminocarbonyl,
   mono-alkylaminocarbonyl,
   dialkylamino,
   3- to 6-membered cycloalkyl,
   unsubstituted or substituted 5- or 6-membered heterocyclyl,
   unsubstituted or substituted 6-membered aryl,
   unsubstituted or substituted 5- or 6-membered heteroaryl, and
   unsubstituted or substituted bicyclic heteroaryl,
wherein a substituted heterocyclyl, aryl, heteroaryl and bicyclic heteroaryl group can optionally be substituted with 1, 2 or 3 substituents independently selected from
   halogen,
   C₁-C₃-alkyl,
   C₁-C₃-haloalkyl,
   C₁-C₃-alkoxy,
   aminocarbonyl, and
   mono-alkylaminocarbonyl,
wherein a mono-alkylaminocarbonyl group may carry a further substituent on the mono-alkyl-chain, selected from halogen-substituted 5- or 6-membered heteroaryl;
and in formulae (b-2) and (b-3) one of D1, D2 and D3 is present and represents
   a fused phenyl ring,
   a fused 6-membered heteroaryl ring,
   a fused 6-membered cycloalkyl ring, or
   a fused 5- or 6-membered heterocyclyl ring;
and the groups (b-2) and (b-3) carry 0 or 1 substituent selected from
   halogen,
   linear or branched C₁-C₃-alkyl,
   linear or branched C₁-C₃-haloalkyl, and
   linear or branched C₁-C₃-alkoxy.

A further aspect relates to compounds of the formula (I-A), (I-B), (I-C), (I-D), (I-E), (I-F) or (I-G) as defined above, with the following group A

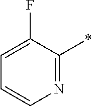 [SMILES: *c1ncccc1F]

and wherein the remaining substituents have the meaning as defined anywhere herein or in context with any of the aspects and embodiments above.

A further aspect relates to compounds of the formula (I-A), (I-B), (I-C), (I-D), (I-E), (I-F) or (I-G) as defined above, with the following group B

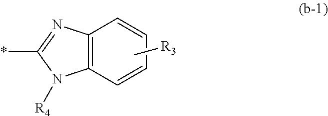

wherein R⁴ represents a substituent as defined anywhere herein excluding hydrogen, and/or wherein R³ represents 1 substituent selected from the above defined group of “cyclic substituents”, and wherein the remaining substituents have the meaning as defined anywhere herein or in context with any of the aspects and embodiments above.

A further aspect relates to compounds of the formula (I-A), (I-B), (I-C), (I-D), (I-E), (I-F) or (I-G) as defined above, with the following group B

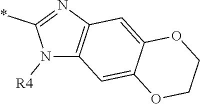

wherein R⁴ represents a substituent as defined anywhere herein, and wherein the remaining substituents have the meaning as defined anywhere herein or in context with any of the aspects and embodiments above.

A further aspect relates to compounds of the formula (I-F) or (I-G) as defined above:

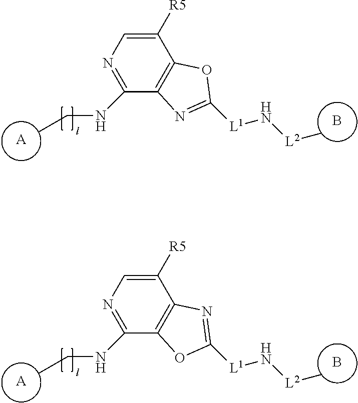

with the following groups A and B:

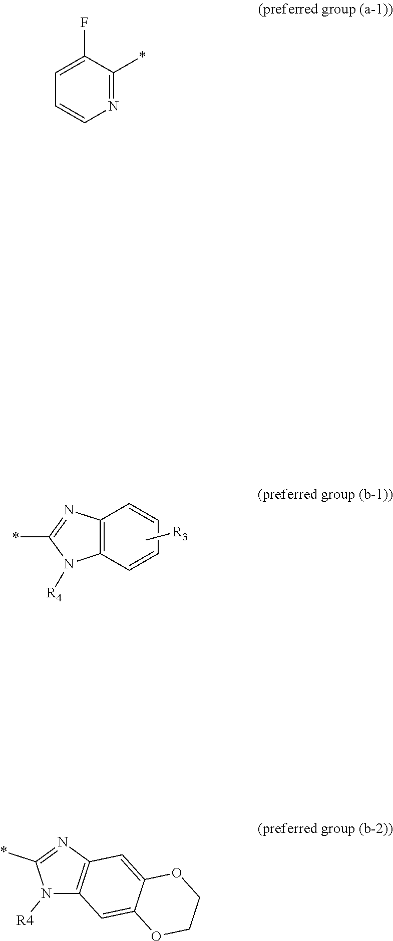

wherein in the above indicated preferred group (b-1) R⁴ represents a substituent as defined anywhere herein excluding hydrogen, and/or wherein R³ represents 1 substituent selected from the above defined group of “cyclic substituents”, and wherein the remaining substituents have the meaning as defined anywhere herein or in context with any of the aspects and embodiments above.

In a further aspect of the invention it is preferred that the compounds of the formula (I-A), (I-B), (I-C), (I-D), (I-E), (I-F) or (I-G) as defined anywhere herein are characterized in that one or more of the substituents defined therein are particularly selected as follows:
halogen substituents are selected from F, Cl and Br; preferably from F and Cl; and/or
linear or branched C₁-C₆-alkyl substituents are selected from methyl, ethyl, propyl, iso-propyl, n-butyl and iso-butyl; preferably from methyl, ethyl and propyl, more preferably from methyl or ethyl; and/or
C₁-C₃-alkoxy substituents are selected from methoxy and ethoxy; and/or
C₁-C₃-haloalkyl substituents are selected from difluoroethyl (—CH₂—CHF₂) and trifluoromethyl (CF₃); and/or
a substituted alkyl-group in the position R⁴ represents a substituted or unsubstituted C₁-C₃-alkyl group, preferably a substituted or unsubstituted C₁- or C₂-alkyl group; and/or
a bicyclic heteroaryl group is selected from a benzimidazolyl group.

In a further aspect of the invention it is preferred that for the compounds of the formula (I-A), (I-B), (I-C), (I-D), (I-E), (I-F) or (I-G) as defined anywhere herein the possible substituents are selected from one or from any combination of two or more of the following definitions:
[1] One of R¹ and R² represents hydrogen and the other one is selected from F, Cl, methyl, ethyl, difluoroethyl, trifluoromethyl, methoxy and ethoxy; preferably from F, Cl, methyl, trifluoromethyl and methoxy, while F is most preferred.
[2]R³ is selected from
   the group of “linear substituents”, comprising:
      linear or branched C₁-C₃-alkyl, preferably ethyl, propyl or iso-propyl;
      linear or branched C₁-C₃-haloalkyl, preferably difluoroethyl or trifluoromethyl;
      linear or branched C₁-C₃-alkoxy, preferably methoxy or ethoxy; or
   the group of “cyclic substituents”, comprising:
      unsubstituted or substituted phenyl;
      unsubstituted or substituted 5- or 6-membered heteroaryl;
      unsubstituted or substituted bicyclic heteroaryl;
      6-membered heterocyclyl;
      6-membered heterocyclylalkyl;
      phenylethinyl; and
      pyridinylethinyl,
   wherein a substituted phenyl, heteroaryl and bicyclic heteroaryl group can optionally be substituted with 1, 2 or 3 substituents independently selected from
      halogen, preferably F or Cl;
      C₁-C₃-alkyl, preferably methyl or ethyl;
      C₁-C₃-haloalkyl, preferably difluoroethyl or trifluoromethyl; and
      C₁-C₃-alkoxy, preferably methoxy or ethoxy;
   wherein R³ is preferably selected from the group of “cyclic substituents”.
[3]R⁴ is selected from
   unsubstituted or substituted 6-membered aryl, preferably phenyl; and
   linear or branched C₁-C₆-alkyl, preferably methyl or ethyl, which may carry 1 substituent selected from C₁-C₃-alkoxy, preferably methoxy or iso-propoxy, from dialkylamino, preferably dimethylamino, from heterocyclyl, preferably morpholinyl or N-substituted C₁-C₃-alkyl piperazin-1-yl, from unsubstituted or substituted 6-membered aryl, preferably phenyl, methoxy-phenyl and chloro-phenyl, and from unsubstituted or substituted 6-membered heteroaryl, preferably pyridinyl; and
   a dialkylether group [R⁶(CH₂)ₓ—O—CH₂)_{y}—], wherein R⁶ represents a C₁-C₃-alkoxy group, preferably a methoxy group; and wherein x and y independently represent an integer of 1, 2 or 3, preferably x and/or y represent 2,
or
R⁴ is selected from
   linear or branched C₁-C₆-alkyl, preferably methyl or ethyl, which may carry 1 substituent selected from C₁-C₃-alkoxy, preferably methoxy or iso-propoxy, from heterocyclyl, preferably morpholinyl, and from unsubstituted or substituted 6-membered aryl, preferably phenyl, methoxy-phenyl and chloro-phenyl; and
   a dialkylether group [R⁶(CH₂)ₓ—O—CH₂)_{y}—], wherein R⁶ represents a C₁-C₃-alkoxy group, preferably a methoxy group; and wherein x and y independently represent an integer of 1, 2 or 3, preferably x and/or y represent 2.
[4] R⁵ is selected from
   hydrogen,
   halogen, preferably F and Cl;
   linear or branched C₁-C₃-alkyl, preferably methyl; and
   linear or branched C₁-C₃-haloalkyl, preferably difluoroethyl and trifluoromethyl.
[5]L¹ and L² independently represent a linker group selected from
   a linear C₁-C₃-alkyl group —[CH₂]ₘ or —[CH₂]ₙ—, respectively, wherein m and n are independently an integer of 1, 2 or 3, preferably m and n are independently 1 or 2, more preferably m and n both represent 2;
   a branched C₁-C₄-alkyl group, preferably a 2-dimethylethyl group; and
   a C₃-C₆-cycloalkyl group which forms a ring together with the nitrogen atom to which it is bonded, preferably a group forming a 4-ring or a 6-ring together with the nitrogen atom to which it is bonded, preferably forming a 4-ring with the nitrogen atom to which they are bonded.

In a particularly preferred aspect the compounds according to the invention are selected from compounds according to formula (I-A), (I-B), (I-C), (I-E) and (I-F) defined above, including in particular the following compounds:

| No. | Compound/Structure |
|---|---|
| 1 | 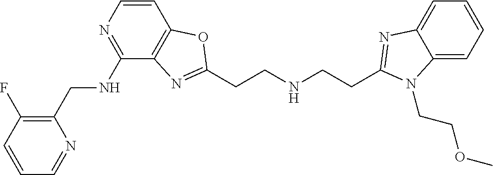 [SMILES: COCCn1c(CCNCCc2nc3c(NCc4ncccc4F)nccc3o2)nc2ccccc21] |
| 2 | 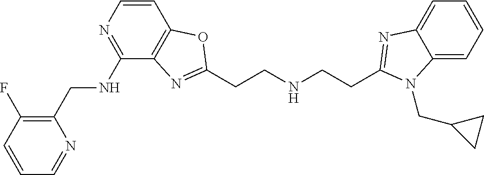 [SMILES: Fc1cccnc1CNc1nccc2oc(CCNCCc3nc4ccccc4n3CC3CC3)nc12] |
| 3 | 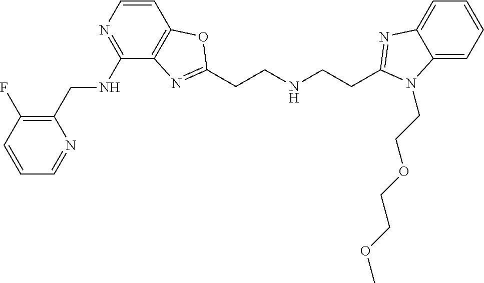 [SMILES: COCCOCCn1c(CCNCCc2nc3c(NCc4ncccc4F)nccc3o2)nc2ccccc21] |
| 4 | 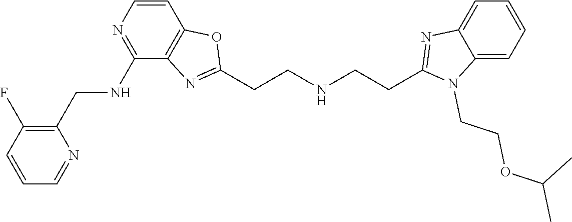 [SMILES: CC(C)OCCn1c(CCNCCc2nc3c(NCc4ncccc4F)nccc3o2)nc2ccccc21] |
| 5 | 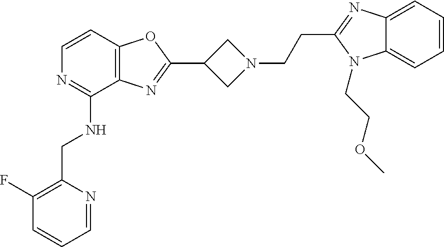 [SMILES: COCCn1c(CCN2CC(c3nc4c(NCc5ncccc5F)nccc4o3)C2)nc2ccccc21] |
| 6 | 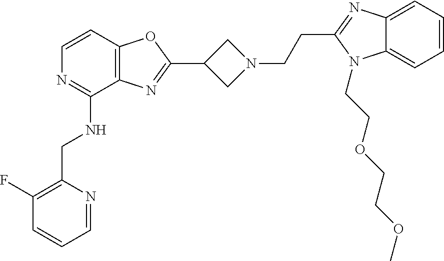 [SMILES: COCCOCCn1c(CCN2CC(c3nc4c(NCc5ncccc5F)nccc4o3)C2)nc2ccccc21] |
| 7 | 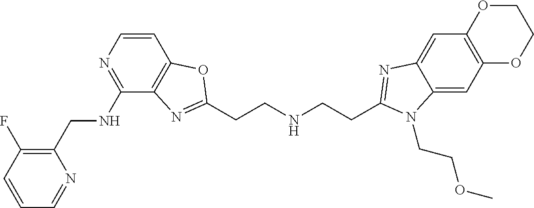 [SMILES: COCCn1c(CCNCCc2nc3c(NCc4ncccc4F)nccc3o2)nc2cc3c(cc21)OCCO3] |
| 8 | 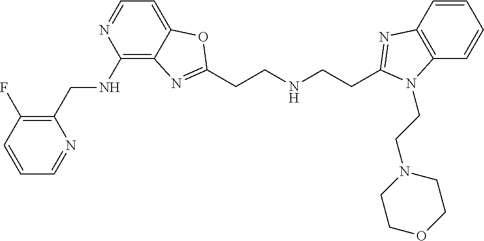 [SMILES: Fc1cccnc1CNc1nccc2oc(CCNCCc3nc4ccccc4n3CCN3CCOCC3)nc12] |
| 9 | 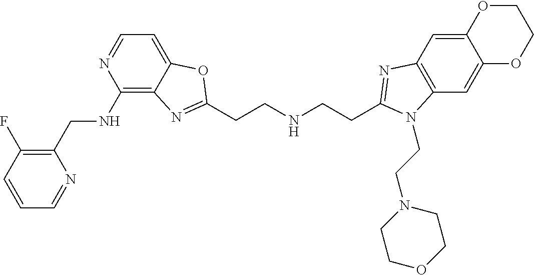 [SMILES: Fc1cccnc1CNc1nccc2oc(CCNCCc3nc4cc5c(cc4n3CCN3CCOCC3)OCCO5)nc12] |
| 10 | 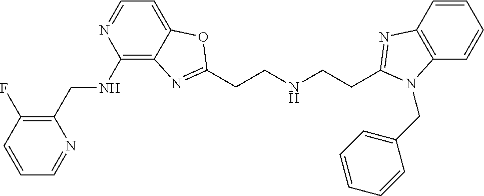 [SMILES: Fc1cccnc1CNc1nccc2oc(CCNCCc3nc4ccccc4n3Cc3ccccc3)nc12] |
| 11 | 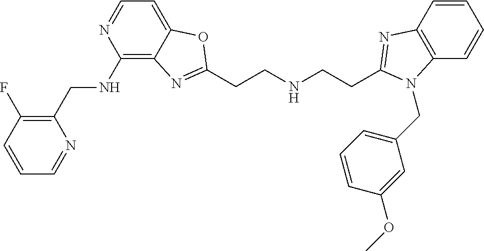 [SMILES: COc1cccc(Cn2c(CCNCCc3nc4c(NCc5ncccc5F)nccc4o3)nc3ccccc32)c1] |
| 12 | 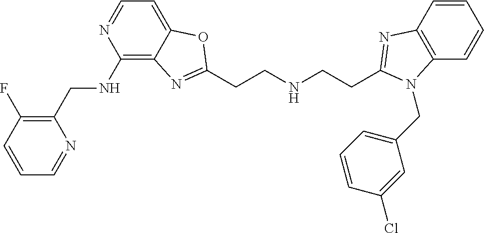 [SMILES: Fc1cccnc1CNc1nccc2oc(CCNCCc3nc4ccccc4n3Cc3cccc(Cl)c3)nc12] |
| 13 | 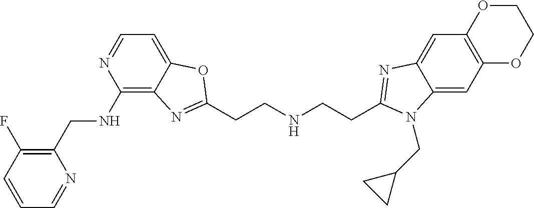 [SMILES: Fc1cccnc1CNc1nccc2oc(CCNCCc3nc4cc5c(cc4n3CC3CC3)OCCO5)nc12] |
| 14 | 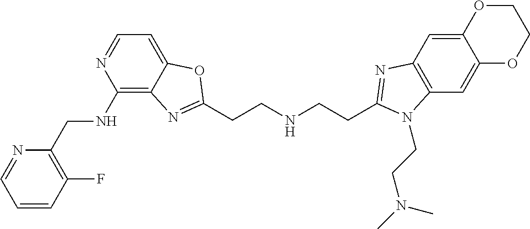 [SMILES: CN(C)CCn1c(CCNCCc2nc3c(NCc4ncccc4F)nccc3o2)nc2cc3c(cc21)OCCO3] |
| 15 | 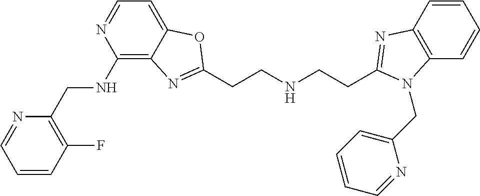 [SMILES: Fc1cccnc1CNc1nccc2oc(CCNCCc3nc4ccccc4n3Cc3ccccn3)nc12] |
| 16 | 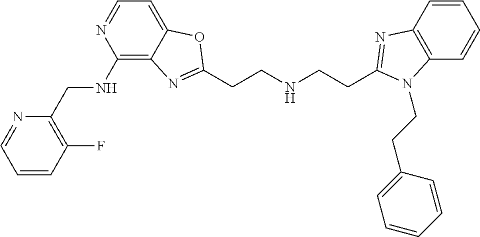 [SMILES: Fc1cccnc1CNc1nccc2oc(CCNCCc3nc4ccccc4n3CCc3ccccc3)nc12] |
| 17 | 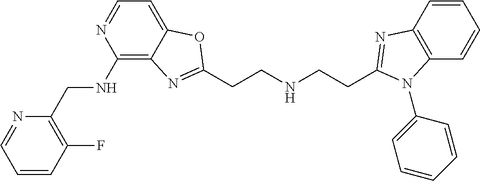 [SMILES: Fc1cccnc1CNc1nccc2oc(CCNCCc3nc4ccccc4n3-c3ccccc3)nc12] |
| 18 | 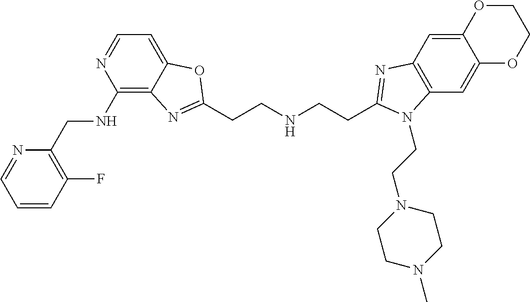 [SMILES: CN1CCN(CCn2c(CCNCCc3nc4c(NCc5ncccc5F)nccc4o3)nc3cc4c(cc32)OCCO4)CC1] |
| 19 | 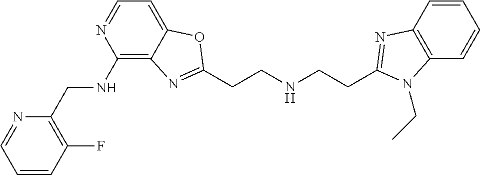 [SMILES: CCn1c(CCNCCc2nc3c(NCc4ncccc4F)nccc3o2)nc2ccccc21] |
| 20 | 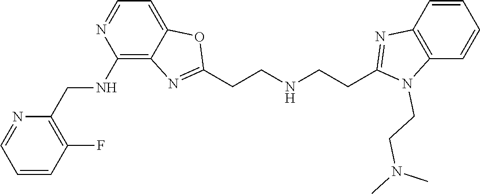 [SMILES: CN(C)CCn1c(CCNCCc2nc3c(NCc4ncccc4F)nccc3o2)nc2ccccc21] |
| 21 | 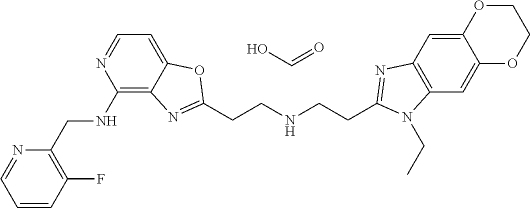 |
| 22 | 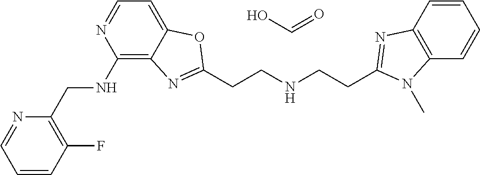 |
| 23 | 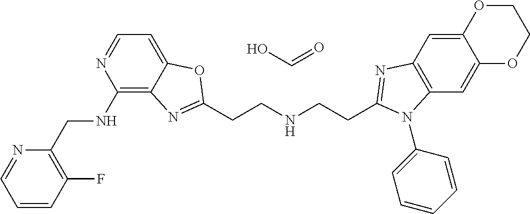 |

In any case, the following compounds from WO2020/123850 are excluded from the invention:

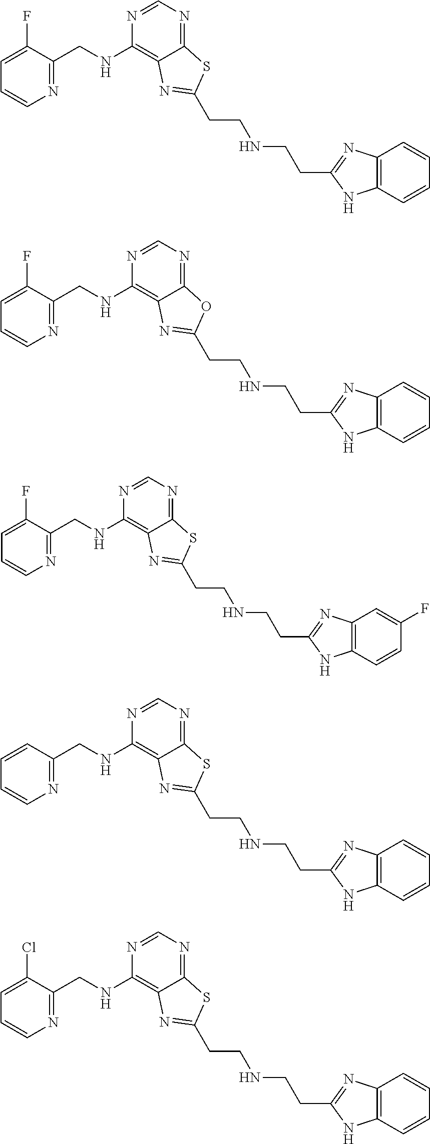 [SMILES: Fc1cccnc1CNc1ncnc2sc(CCNCCc3nc4ccccc4[nH]3)nc12 ; Fc1cccnc1CNc1ncnc2oc(CCNCCc3nc4ccccc4[nH]3)nc12 ; Fc1ccc2[nH]c(CCNCCc3nc4c(NCc5ncccc5F)ncnc4s3)nc2c1 ; c1ccc(CNc2ncnc3sc(CCNCCc4nc5ccccc5[nH]4)nc23)nc1 ; Clc1cccnc1CNc1ncnc2sc(CCNCCc3nc4ccccc4[nH]3)nc12]

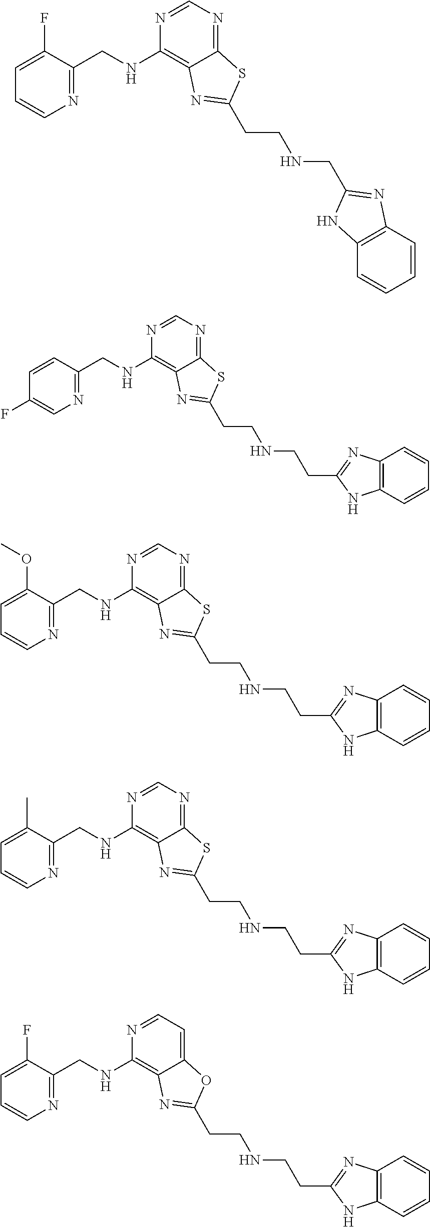 [SMILES: Fc1cccnc1CNc1ncnc2sc(CCNCc3nc4ccccc4[nH]3)nc12 ; Fc1ccc(CNc2ncnc3sc(CCNCCc4nc5ccccc5[nH]4)nc23)nc1 ; COc1cccnc1CNc1ncnc2sc(CCNCCc3nc4ccccc4[nH]3)nc12 ; Cc1cccnc1CNc1ncnc2sc(CCNCCc3nc4ccccc4[nH]3)nc12 ; Fc1cccnc1CNc1nccc2oc(CCNCCc3nc4ccccc4[nH]3)nc12]

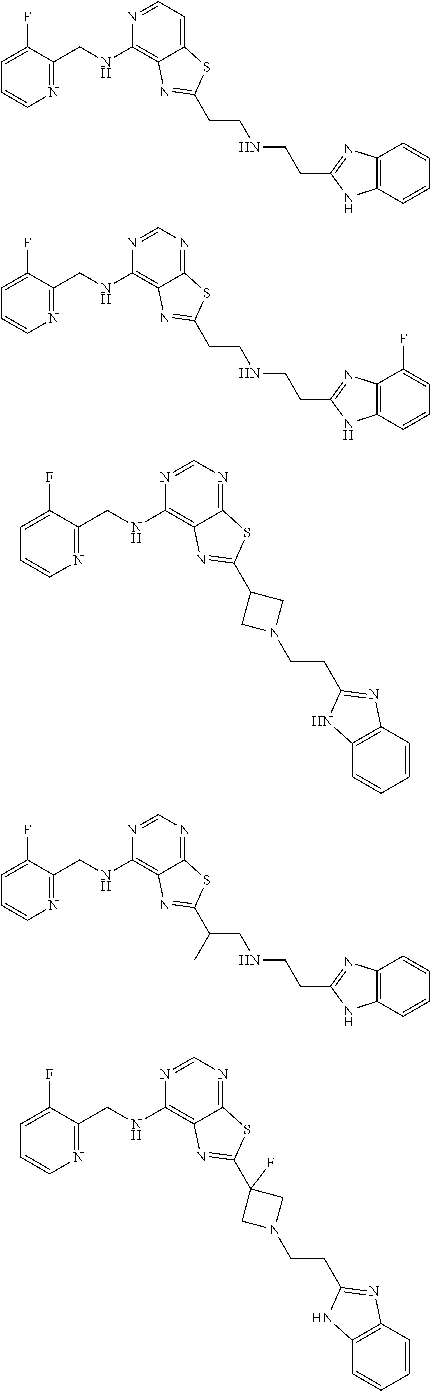 [SMILES: Fc1cccnc1CNc1nccc2sc(CCNCCc3nc4ccccc4[nH]3)nc12 ; Fc1cccnc1CNc1ncnc2sc(CCNCCc3nc4c(F)cccc4[nH]3)nc12 ; Fc1cccnc1CNc1ncnc2sc(C3CN(CCc4nc5ccccc5[nH]4)C3)nc12 ; CC(CNCCc1nc2ccccc2[nH]1)c1nc2c(NCc3ncccc3F)ncnc2s1 ; Fc1cccnc1CNc1ncnc2sc(C3(F)CN(CCc4nc5ccccc5[nH]4)C3)nc12]

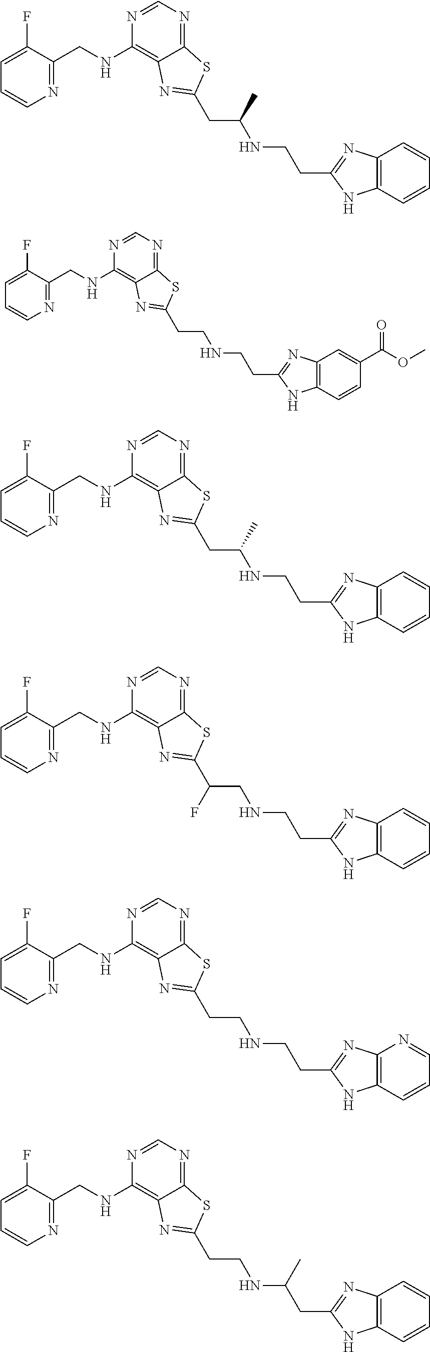 [SMILES: C[C@H](Cc1nc2c(NCc3ncccc3F)ncnc2s1)NCCc1nc2ccccc2[nH]1 ; COC(=O)c1ccc2[nH]c(CCNCCc3nc4c(NCc5ncccc5F)ncnc4s3)nc2c1 ; C[C@@H](Cc1nc2c(NCc3ncccc3F)ncnc2s1)NCCc1nc2ccccc2[nH]1 ; Fc1cccnc1CNc1ncnc2sc(C(F)CNCCc3nc4ccccc4[nH]3)nc12 ; Fc1cccnc1CNc1ncnc2sc(CCNCCc3nc4ncccc4[nH]3)nc12 ; CC(Cc1nc2ccccc2[nH]1)NCCc1nc2c(NCc3ncccc3F)ncnc2s1]

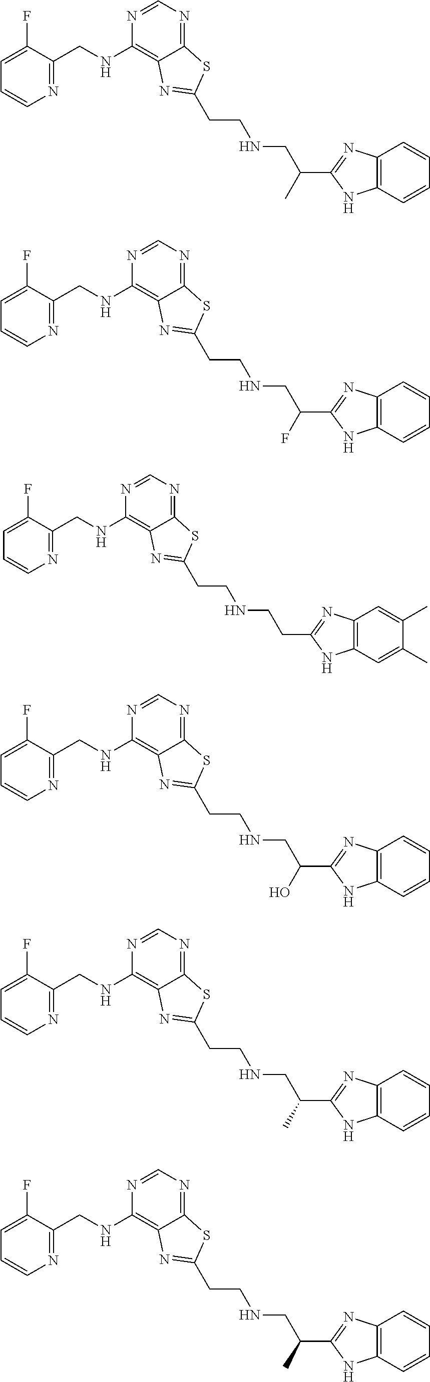 [SMILES: CC(CNCCc1nc2c(NCc3ncccc3F)ncnc2s1)c1nc2ccccc2[nH]1 ; Fc1cccnc1CNc1ncnc2sc(CCNCC(F)c3nc4ccccc4[nH]3)nc12 ; Cc1cc2nc(CCNCCc3nc4c(NCc5ncccc5F)ncnc4s3)[nH]c2cc1C ; OC(CNCCc1nc2c(NCc3ncccc3F)ncnc2s1)c1nc2ccccc2[nH]1 ; C[C@H](CNCCc1nc2c(NCc3ncccc3F)ncnc2s1)c1nc2ccccc2[nH]1 ; C[C@@H](CNCCc1nc2c(NCc3ncccc3F)ncnc2s1)c1nc2ccccc2[nH]1]

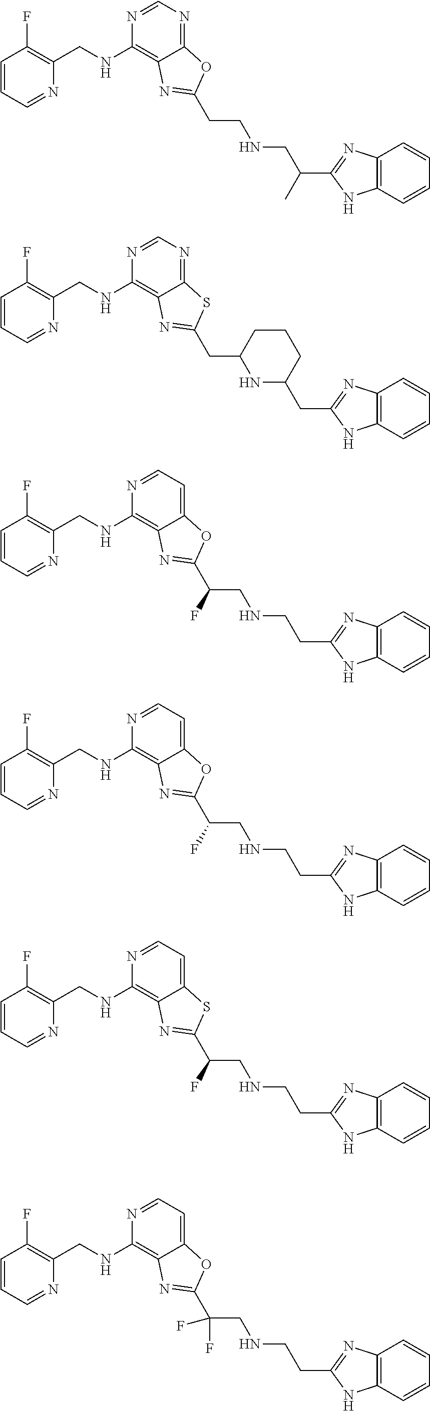 [SMILES: CC(CNCCc1nc2c(NCc3ncccc3F)ncnc2o1)c1nc2ccccc2[nH]1 ; Fc1cccnc1CNc1ncnc2sc(CC3CCCC(Cc4nc5ccccc5[nH]4)N3)nc12 ; Fc1cccnc1CNc1nccc2oc([C@H](F)CNCCc3nc4ccccc4[nH]3)nc12 ; Fc1cccnc1CNc1nccc2oc([C@@H](F)CNCCc3nc4ccccc4[nH]3)nc12 ; Fc1cccnc1CNc1nccc2sc([C@H](F)CNCCc3nc4ccccc4[nH]3)nc12 ; Fc1cccnc1CNc1nccc2oc(C(F)(F)CNCCc3nc4ccccc4[nH]3)nc12]

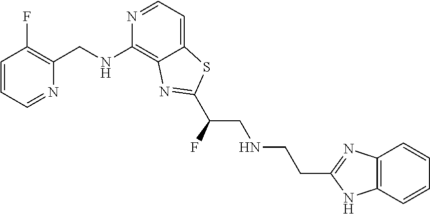 [SMILES: Fc1cccnc1CNc1nccc2sc([C@H](F)CNCCc3nc4ccccc4[nH]3)nc12]

The present invention relates to the novel ferroportin inhibitor compounds as defined anywhere herein, including pharmaceutically acceptable salts thereof. In a further aspect, the invention also includes solvates, hydrates and polymorphs of the compounds and their pharmaceutically acceptable salts as defined herein.

The salts, solvates or hydrates of the compounds of the formulae (I-A), (I-B), (I-C), (I-E), (I-F), or (I-G) as defined anywhere herein may be present in amorphous, polymorphous, crystalline and/or semi-crystalline (partly crystalline) form as well as in the form of a solvate (or hydrate) of a salt. Preferably the salts, solvates or hydrates of the present invention are present in crystalline and/or semi-crystalline (partly crystalline) form.

The preferred crystallinity of the salts or salt solvates of the present invention can be determined by using conventional analytical methods, such as especially by using the various X-ray methods, which permit a clear and simple analysis of the salt compounds. In particular, the grade of crystallinity can be determined or confirmed by using Powder X-ray diffraction (reflection) methods as described for example in the Examples below, or by using Powder X-ray diffraction (transmission) methods as described for example in the Examples below (both being hereinafter also abbreviated as PXRD). For crystalline solids having identical chemical composition, the different resulting crystal gratings are summarized by the term polymorphism.

Pharmaceutically acceptable salts of the compounds according to the invention include, for example, salts with suitable anions, such as carboxylates which may include formates, or acetates but also sulfonates, sulfates, chlorides, bromides, iodides, phosphates, tartrates, methane sulfonates, hydroxyethane sulfonates, glycinates, maleates, propionates, fumarates, toluene sulfonates, benzene sulfonates, trifluoroacetates, naphthalenedisulfonates-1,5, salicylates, benzoates, lactates, salts of malic acid, salts of 3-hydroxy-2-naphthoic acid-2, citrates and acetates. HCl salts are preferred.

Pharmaceutically acceptable salts of the compounds according to the invention further include, for example, salts with suitable pharmaceutically acceptable bases, such as, for example, salts with alkaline or alkaline-earth hydroxides, such as NaOH, KOH, Ca(OH)₂, Mg(OH)₂ etc., amine compounds such as ethylamine, diethylamine, triethylamine, ethyldiisopropylamine, ethanolamine, diethanolamine, triethanolamine, methylglucamine, dicyclohexylamine, dimethylaminoethanol, procaine, dibenzylamine, N-methylmorpholine, arginine, lysine, ethylenediamine, N-methylpiperidin, 2-amino-2-methyl-propanol-(1), 2-amino-2-methyl-propandiol-(1,3), 2-amino-2-hydroxyl-methyl-propandiol-(1,3) (TRIS) etc.

The novel compounds of the present invention may be present as solvates and/or hydrates, which may be formed by attraction, association, adsorption, adhesion, embedding or complexation of molecules of a solvent in the crystal grating of the salts of the present invention. The solvent molecules which may be embedded in the crystal grating may derive from the solvents used for crystallization as well as from water deriving from the relative humidity.

The extent to which a selected solvent or water leads to a solvate or hydrate in the process steps or during the crystallization step depends on the combination of process conditions and the various interactions between the selected compound, the counter anion from the selected acid and the selected solvent and humidity conditions. The salt solvates or hydrates may be preferred, as solvent or water molecules in the crystal structure are bound by strong intermolecular forces and thereby may represent an element of structure formation of these crystals which, in part, may improve stability of the salt. However, solvent and/or water molecules are also existing in certain crystal lattices which are bound by rather weak intermolecular forces. Such molecules are more or less integrated in the crystal structure forming, but to a lower energetic effect. The solvent and/or water content of the solvates is also dependent on the drying and ambient conditions (i.e. relative humidity). In the case of stable solvates or hydrates, there are usually clear stoichiometric ratios between the active compound (i.e. the salt) and the solvent or water. In many cases these ratios do not fulfil completely the stoichiometric value, normally it is approached by lower values compared to theory because of certain crystal defects. The ratio of organic molecules to solvent or water molecules for the weaker bound water may vary to a considerable extend, for example, extending over di-, tri- or tetra-hydrates. On the other hand, in amorphous solids, the molecular structure classification of solvent and/or water is not stoichiometric; the classification may however also be stoichiometric only by chance. In some cases, it is not possible to classify the exact stoichiometry of the solvent or water molecules, since layer structures form so that the embedded solvent or water molecules cannot be determined in defined form.

The solvent and/or water content in amorphous solids as well as in crystalline solvates or hydrates can, in general, be determined by conventional methods, such as e.g. by using the well-known Karl-Fischer titration method, by carrying out dynamic vapor sorption (DVS) measurements, by carrying out thermogravimetric measurements (TG-FTIR). Also elemental analysis or methods for structural analysis, such as ¹H NMR spectroscopy or Raman spectroscopy (FT Raman spectroscopy) may give information about the degree of solvate or hydrate formation and/or may be used to confirm or validate the results of the Karl-Fischer (KF), DVS or TG-FTIR measurements.

Examples of solvates and/or hydrates according to the present invention comprise for example, hemi- (0.5), mono-, sesqui- (1.5), di-, tri-, tetra-, penta-, hexa-, hepta-, octa-, nona-deca-, etc. solvates or hydrates, respectively. Further intermediate solvation-degrees are also possible, such as solvation with 2.5, 3.5, 4.5 etc. solvent and/or water molecules.

Preferred examples of solvates and/or hydrates comprise solvates/hydrates with about 0.5, 1, 1.5, 2.5, 3, 4 and 7 solvate/water molecules. Further preferred examples of solvates and/or hydrates comprise solvates/hydrates with about 0.5, 1, 1.5, 2.5, 3, 4, 6 and 7 solvate/water molecules. More preferred are hemi- and mono- solvates/hydrates with about 0.5 or 1 solvate/water molecules, wherein hemi- and mono-hydrates are particularly preferred. Anhydrous salts are also preferred. It is further possible, that solvent and/or water residues remain in the salt in non-stoichiometric amounts.

The formation of the salts of the compounds of the present invention can in particular be carried out by the methods described in the international application WO2018/192973.

As described therein, solvents used for crystallization comprise acetonitrile, dichloromethane (DCM), alcohols, such as especially methanol, ethanol, 2-propanol (iso-propanol), aldehydes, ketones, especially acetone, ethers, e.g. tetrahydrofuran (THF) or dioxane, esters, e.g. ethyl acetate, or alkanes, such as especially pentane, hexane, heptane or cyclohexane and water, and mixtures thereof. Preferred solvents used for crystallization are selected from the group consisting of acetonitrile, dichloromethane, methanol, ethanol, 2-propanol, ethyl acetate, THF, water and mixtures thereof.

Particularly preferred solvents used for crystallization are selected from the group consisting of acetonitrile, methanol, ethanol, 2-propanol, ethyl acetate, THF, water and mixtures thereof. Preferred water/solvent mixtures comprise mixtures of water, acetonitrile, and methanol or ethanol, wherein mixtures of water, acetontrile and methanol are preferred.

In principle, the salts, solvates, hydrates, and polymorphs of the compounds according to the present invention may be prepared by any commonly applied crystallization technique including vaporization crystallization, cooling crystallization, crystallization by addition of an antisolvent, seeding crystallization, template crystallization or crystallization from a melt.

In an especially preferred embodiment, salts, solvates, or hydrates of the compounds as defined anywhere herein are prepared using the especially preferred solvent mixtures in preparative high-performance liquid chromatography (HPLC). Preparation of salts, solvates or hydrates in this manner has the advantage that reaction products from compound syntheses can be purified while the compounds according to the invention can simultaneously be transferred to a desired phase. In such cases, suitable anions may be selected from formate, acetate or lactate. The respective anions are added to the HPLC solution by 0.05 w. %, 0.075 w. %, 0.1 w. %, 0.2 w. %, 0.3 w. %, 0.4 w. % or 0.5 w. %, preferred are 0.1 w. %. Subsequently, the preparative HPLC may be performed.

The novel compounds according to formula (I-A), (I-B), (I-C), (I-D), (I-E), (I-F) or (I-G) as defined anywhere herein, have been found to act as ferroportin inhibitors and are thus suitable for the use as a medicament, such as in particular for the use as ferroportin inhibitors.

As already explained above, ferroportin is the iron transport protein, which is responsible for the uptake of the released iron via the intestine and its transfer into the blood circulation, thereby conveying the iron to the appropriate tissues and organs. Inactivation or inhibition of the ferroportin disables the export of the iron, thereby reducing the absorption of iron in the intestine. Ferroportin inhibition in the sense of the present invention therefore includes the inhibition of iron transport from the cells into the blood circulation and the inhibition of iron absorption in the intestine. Therein, the inhibition of iron transport and/or iron reflux may be effected by different ways of mechanism, comprising for example inhibition of iron transport activity of ferroportin and thus inhibition of iron reflux, triggering internalization, degradation and/or reduction of ferroportin, administering hepcidin agonists, i.e. compounds which compete with hepcidin or by compounds, which inhibit the binding of hepcidin to ferroportin.

Ferroportin inhibition may be determined by measuring the inhibition of ferroportin mediated iron transport activity in an iron response assay (BLAzer-Assay), as described in more detail in the Examples below. Further, ferroportin inhibition may be determined by measuring ferroportin internalization and/or degradation in the Ferroportin Internalization and Degradation Assay (FACS) or by examining the Ferroportin Ubiquitination and Degradation, each as described in more detail in the Examples below. Further, ferroportin inhibition may be determined by measuring the activity as an hepcidin agonist, for example by determining the Hepcidin binding capacity to ferroportin in the Hepcidin Internalization Assay (J774), as described in more detail in the Examples below. Further, ferroportin inhibition may be determined by confirming the inhibition of hepcidin binding to ferroportin, for example in the Biophysical Ferroportin-Hepcidin Binding Assay (Hep Bind FP), as described in more detail in the Examples below. Further, ferroportin inhibition may be determined by determining the activity of a compound regarding its ability to block iron export via ferroportin, for example with a test for measuring inhibition of iron efflux, as described in more detail in the Examples below.

Ferroportin inhibition in the sense of the present invention can thus in particular be defined by exhibiting a ferroportin inhibiting activity in at least one of the aforementioned test methods, shown in particular by:
Inhibition of ferroportin mediated iron transport activity in an iron response assay (Blazer Assay): IC₅₀ value [μM] of not more than 100 (≤100), preferably not more than 50 (≤50), more preferably below 50 (<50).
Ferroportin Internalization and Degradation Assay (FACS): EC₅₀ value [μM] of not more than 100 (≤100), preferably not more than 50 (≤50), more preferably below 50 (<50).
Ferroportin Ubiquitination and Degradation: visually inspected effect in Western blots of “+ comparable to hepcidin”, “+/− intermediate effect” and “+/+/− stronger intermediate effect”, preferred is an effect “+” or “+/+/−”, most preferred is an effect “+”.
Hepcidin Internalization Assay (J774): IC₅₀ value [μM] of not more than 100 (≤100), preferably not more than 50 (≤50), more preferably below 50 (<50).
Biophysical Ferroportin-Hepcidin Binding Assay: IC₅₀ value [μM] of not more than 100 (≤100), preferably not more than 50 (≤50), more preferably below 50 (<50).
Inhibition of Iron Efflux: IC₅₀ value of not more than 100 (≤100), preferably not more than 50 (≤50), more preferably below 50 (<50).

Ferroportin inhibition may further be determined in in vivo models, as described in more detail in the Examples below. Suitable in vivo models may comprise, for example, examination of hypoferremia in naïve mice via measurement of serum iron reduction; examination of prevention of iron absorption in anemic rats via measurement of serum iron inhibition; examination of correction of hyperferremia in beta2-microglobulin deficient mice via measurement of serum iron reduction; examination of prevention of iron overload in beta2-microglobulin deficient mice via measurement of total iron in spleen or liver; examination of improvement of anemia, ineffective erythropoiesis and iron overload in a mouse model of β-thalassemia intermedia.

The activity of the compounds of the present invention as ferroportin inhibitors can in particular be determined by the methods as described in the Examples below.

As further already explained above, ferroportin inhibition may for example be effected by hepcidin, which is thus an essential regulating factor of iron absorption, inhibiting ferroportin and thus blocking iron transport from the cells into the blood circulation and iron absorption. It has further been found that several of the compounds as defined herein act as hepcidin mimetics or hepcidin agonists, which is also included by ferroportin inhibition in the sense of the present invention.

Accordingly, the compounds as defined in the present invention are also suitable for use in the inhibition of iron transport from the cells into the blood circulation and the inhibition of iron absorption in the intestine, as well as for the use as hepcidin mimetics or hepcidin agonists.

Due to the activity of the compounds as defined herein as ferroportin inhibitors, the compounds of the present invention are further particularly suitable for the use in the inhibition of iron transport mediated by ferroportin and thereby for the use in the prophylaxis and/or treatment of iron metabolism disorders leading to increased iron levels, of diseases related to or caused by increased iron levels, increased iron absorption or iron overload, such as in particular of tissue iron overload, of diseases associated with ineffective erythropoiesis, or of diseases caused by reduced levels of hepcidin. Further, the compounds of the present invention are suitable for the use in an adjunctive therapy by limiting the amount of iron available to pathogenic microorganisms, such as the bacterium Vibrio vulnificus, thereby preventing or treating infections caused by said pathogenic microorganisms.

Therein, diseases being associated with, being related to, being caused by or leading to increased iron levels, increased iron absorption, iron overload (e.g. tissue iron overload) or ineffective erythropoiesis comprise thalassemia, hemoglobinopathy, such as hemoglobin E disease (HbE), hemoglobin H disease (HbH), haemochromatosis, hemolytic anemia, such as sickle cell anemia (sickle cell disease) and congenital dyserythropoietic anemia.

Diseases being associated with, being related to, being caused by or leading to increased iron levels, increased iron absorption, iron overload (e.g. tissue iron overload) further comprise neurodegenerative diseases, such as for example Alzheimer's disease and Parkinson's disease, wherein the compounds are considered to be effective by limiting the deposition or increase of iron in tissue or cells.

The compounds of the present invention are further suitable for the use in the prophylaxis and/or treatment of formation of radicals, reactive oxygen species (ROS) and oxidative stress caused by excess iron or iron overload as well as in the prophylaxis and/or treatment of cardiac, liver and endocrine damage caused by excess iron or iron overload, and further in the prophylaxis and/or treatment of inflammation triggered by excess iron or iron overload.

Diseases associated with ineffective erythropoiesis comprise in particular myelodysplastic syndromes (MDS, myelodysplasia) and polycythemia vera as well as congenital dyserythropoietic anemia.

Further diseases, disorders and/or diseased conditions comprise iron overload caused by mutations in genes involved in sensing the systemic iron stores, such as hepcidin (Hamp1), hemochromatosis protein (HFE), hemojuvelin (HJV) and transferrin receptor 2 (TFR2), such as in particular diseases related to HFE and HJV gene mutations, chronic hemolysis associated diseases, sickle cell diseases, red cell membrane disorders, Glucose-6-phosphate dehydrogenase deficiency (G6PD deficiency), erythropoietic porphyria, Friedrich's Ataxia, as well as subgroups of iron overload such as transfusional iron overload, iron intoxication, pulmonary hemosiderosis, osteopenia, insulin resistance, African iron overload, Hallervorden-Spatz disease, hyperferritinaemia, ceruloplasmin deficiency, neonatal hemochromatosis and red blood cell disorders comprising thalassemia, including alpha thalassemia, beta thalassemia and delta thalassemia, thalassemia intermedia, sickle cell disease and myelodysplastic syndrome.

Further diseases and/or disorders and/or diseased conditions associated with elevated iron levels include, but are not limited to, diseases with elevated iron level, comprising ataxia, Friedrich's ataxia, age-related macular degeneration, age-related cataract, age-related retinal diseases and neurodegenerative disease, such as pantothenate kinase-associated neurodegeneration, restless leg syndrome and Huntington's disease,

The compounds of the present invention my further be suitable for the use in the prophylaxis and treatment of diseases caused by a lack of hepcidin.

In view thereof a further object of the present invention relates to a medicament containing one or more of the compounds as defined above, such as in particular a medicament for the prophylaxis and treatment in any of the indications, states, disorders or diseases as defined above.

A further object of the present invention relates to pharmaceutical compositions and medicaments comprising one or more of the compounds according to the invention as defined above as well as optionally one or more pharmacologically acceptable carriers and/or auxiliary substances and/or solvents. A further object of the present invention relates to pharmaceutical compositions and medicaments comprising one or more of the compounds according to the invention as defined above as well as optionally one or more further pharmaceutically effective compounds. The said pharmaceutical compositions contain, for example up to 99 weight-% or up to 90 weight-% or up to 80 weight-% or or up to 70 weight-% of the compounds of the invention, the remainder being each formed by pharmacologically acceptable carriers and/or auxiliaries and/or solvents and/or optionally further pharmaceutically active compounds.

Therein, the pharmaceutically acceptable carriers, auxiliary substances or solvents are common pharmaceutical carriers, auxiliary substances or solvents, including various organic or inorganic carrier and/or auxiliary materials as they are customarily used for pharmaceutical purposes, in particular for solid medicament formulations. Examples include excipients, such as saccharose, starch, mannitol, sorbitol, lactose, glucose, cellulose, talcum, calcium phosphate, calcium carbonate; binding agents, such as cellulose, methylcellulose, hydroxypropylcellulose, polypropyl pyrrolidone, gelatine, gum arabic, polyethylene glycol, saccharose, starch; disintegrating agents, such as starch, hydrolyzed starch, carboxymethylcellulose, calcium salt of carboxymethylcellulose, hydroxypropyl starch, sodium glycol starch, sodium bicarbonate, calcium phosphate, calcium citrate; lubricants, such as magnesium stearate, talcum, sodium laurylsulfate; flavorants, such as citric acid, menthol, glycin, orange powder; preserving agents, such as sodium benzoate, sodium bisulfite, paraben (for example methylparaben, ethylparaben, propylparaben, butylparaben); stabilizers, such as citric acid, sodium citrate, acetic acid and multicarboxylic acids from the titriplex series, such as, for example, diethylenetriaminepentaacetic acid (DTPA); suspending agents, such as methycellulose, polyvinyl pyrrolidone, aluminum stearate; dispersing agents; diluting agents, such as water, organic solvents; waxes, fats and oils, such as beeswax, cocoa butter; polyethylene glycol; white petrolatum; etc.

Liquid medicament formulations, such as solutions, suspensions and gels usually contain liquid carrier, such as water and/or pharmaceutically acceptable organic solvents. Furthermore, such liquid formulations can also contain pH-adjusting agents, emulsifiers or dispersing agents, buffering agents, preserving agents, wetting agents, gelatinizing agents (for example methylcellulose), dyes and/or flavouring agents, for example as defined above. The compositions may be isotonic, that is, they can have the same osmotic pressure as blood. The isotonicity of the composition can be adjusted by using sodium chloride and other pharmaceutically acceptable agents, such as, for example, dextrose, maltose, boric acid, sodium tartrate, propylene glycol and other inorganic or organic soluble substances. The viscosity of the liquid compositions can be adjusted by means of a pharmaceutically acceptable thickening agent, such as methylcellulose. Other suitable thickening agents include, for example, xanthan gum, carboxymethylcellulose, hydroxypropylcellulose, carbomer and the like. The preferred concentration of the thickening agent will depend on the agent selected.

Pharmaceutically acceptable preserving agents can be used in order to increase the storage life of the liquid composition. Benzyl alcohol can be suitable, even though a plurality of preserving agents including, for example, paraben, thimerosal, chlorobutanol and benzalkonium chloride can also be used.

The above-mentioned pharmaceutical compositions are suitable, for example, for intravenous, intraperitoneal, intramuscular, intravaginal, intrabuccal, percutaneous, subcutaneous, mucocutaneous, oral, rectal, transdermal, topical, intradermal, intragasteral or intracutaneous application and are provided, for example, in the form of pills, tablets, enteric-coated tablets, film tablets, layer tablets, sustained release formulations for oral, subcutaneous or cutaneous administration (in particular as a plaster), depot formulations, dragees, suppositories, gels, salves, syrup, granulates, suppositories, emulsions, dispersions, microcapsules, microformulations, nanoformulations, liposomal formulations, capsules, enteric-coated capsules, powders, inhalation powders, microcrystalline formulations, inhalation sprays, epipastics, drops, nose drops, nose sprays, aerosols, ampoules, solutions, juices, suspensions, infusion solutions or injection solutions etc.

A further aspect of the present invention relates to medicaments or combined preparations containing one or more of the compounds as defined above and at least one further pharmaceutically active compound, such as in particular a compound for the prophylaxis and treatment of iron overload and the associated symptoms, preferably an iron-chelating compound, or a compound for the prophylaxis and treatment of any of the states, disorders or diseases as defined above, such as in particular a pharmaceutically active compound for the prophylaxis and treatment of thalassemia, haemochromatosis, neurodegenerative diseases (such as Alzheimer's disease or Parkinson's disease) and the associated symptoms.

A further aspect of the present invention relates to the use of the compounds as defined above per se, in a combination therapy (fixed dose or free dose combinations for sequential use) with one or two other active ingredients (drugs). Such combination therapy comprises co-administration of the compounds of the present invention with the at least one additional pharmaceutically active compound (drug). Combination therapy in a fixed dose combination therapy comprises co-administration of the compounds of the present invention with the at least one additional pharmaceutically active compound in a fixed-dose formulation. Combination therapy in a free dose combination therapy comprises co-administration of the compounds of the present invention and the at least one additional pharmaceutically active compound in free doses of the respective compounds, either by simultaneous administration of the individual compounds or by sequential use of the individual compounds distributed over a time period. The at least one additional pharmaceutically active compound (drug) comprises in particular drugs for reducing iron overload (e.g. Tmprss6-ASO) or iron chelators, in particular curcumin, SSP-004184, Deferitrin, deferasirox, deferoxamine and/or deferiprone, or antioxidants such as n-acetyl cysteine, anti-diabetics such as GLP-1 receptor agonists, antibiotics such as vancomycin (Van) or tobramycin, drugs for the treatment of malaria, anticancer agents, antifungal drugs, drugs for the treatment of neurodegenerative diseases such as Alzheimer's disease and Parkinson's disease (e.g. dopamine agonists such as Levodopa), anti-viral drugs such as interferon-α or ribavirin, or immunosuppressants (cyclosporine A or cyclosporine A derivatives), iron supplements, vitamin supplements, red cell production stimulators, anti-inflammatory biologies, anti-thrombolytics, statins, vasopressors and inotropic compounds.

A further aspect of the present invention relates to the use of the above combinations for the prophylaxis and/or treatment of diseases caused by a lack of hepcidin or iron metabolism disorders, such as particularly iron overload states such as in particular thalassemia and hemochromatosis and other disorders as described in the present application.

A further aspect of the present invention relates to the use of the compounds as defined herein per se or the hereinabove described combination therapies, in combination with Blood transfusion.

The compounds, medicaments and or combined preparations according to the present invention may be administered orally, parentally, as well as intravenously.

For this purpose, the compounds according to the invention are preferably provided in medicaments or pharmaceutical compositions in the form of pills, tablets, such as enteric-coated tablets, film tablets and layer tablets, sustained release formulations for oral administration, depot formulations, dragees, granulates, emulsions, dispersions, microcapsules, microformulations, nanoformulations, liposomal formulations, capsules, such as enteric-coated capsules, powders, microcrystalline formulations, epipastics, drops, ampoules, solutions, suspensions, infusion solutions or injection solutions or in the form of a preparation suitable for inhalation.

In a preferred embodiment of the invention the compounds are administered in the form of a tablet or capsule, as defined above. These may be present, for example, as acid resistant forms or with pH dependent coatings.

The compounds of the present invention as the active substance can be administered, for example, with a unit dose of 0.001 mg/kg to 500 mg/kg body weight, for example 1 to 4 times a day. However, the dose can be increased or reduced depending on the age, weight, condition of the patient, severity of the disease or type of administration.

Accordingly, a further aspect of the present invention relates to compounds, medicaments, compositions and combined preparations as defined above for the preparation of a medicament, particularly for the prophylaxis and treatment of any indication, state, disorder or disease as defined above, in particular for oral or parenteral administration.

A further aspect of the present invention relates to a method for the prophylaxis and treatment as defined above, such as in particular for the prophylaxis and/or treatment of iron metabolism disorders being associated with or leading to increased iron levels and in particular iron overload, diseases related to or caused by increased iron levels or iron overload, iron storage diseases being associated with or leading to increased iron levels, and diseases being associated with ineffective erythropoiesis, the method comprising administering, to a patient (human or animal) in need thereof, a compound, a medicament, a composition or a combined preparation as defined above.

Therein, diseases being associated with, being related to, being caused by or leading to increased iron levels or iron overload are as defined above.

A further aspect of the present invention relates to the use of the compounds as defined above for the preparation of a medicament, particularly for the prophylaxis and treatment and of any indication, state, disorder or disease as defined above.

The compounds according to the invention of general structural formula (I-A) and (I-B) can basically be prepared as shown in the following general synthesis schemes I, II and III:

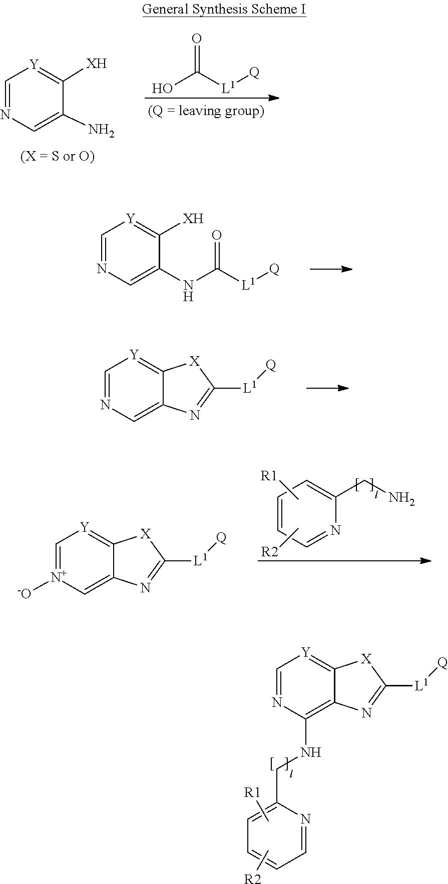

(wherein the leaving group Q may be NHBoc or NBoc and wherein the position of “XH” and “NH₂” can be exchanged to prepare the so-called “iso-compounds” as described herein).

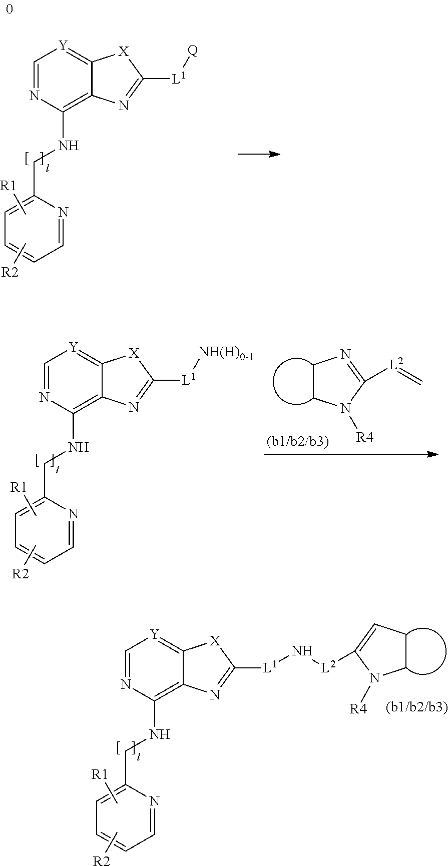

(wherein the structure indicated as “(b1/b2/b3)” represents the structures b-1, b-2 or b-3 as defined herein).

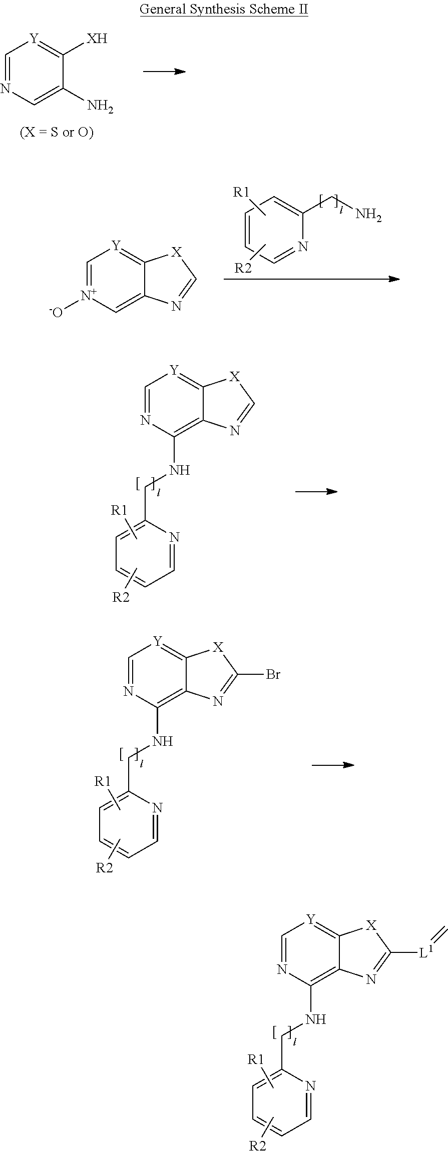

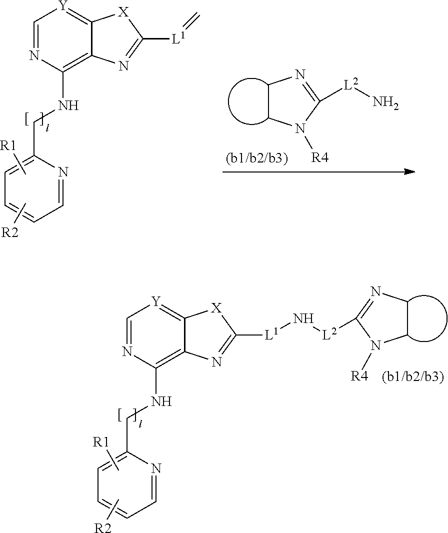

(wherein the position of “XH” and “NH₂” can be exchanged to prepare the so-called “iso-compounds” as described herein and wherein the structure indicated as “(b1/b2/b3)” represents the structures b-1, b-2 or b-3 as defined herein)

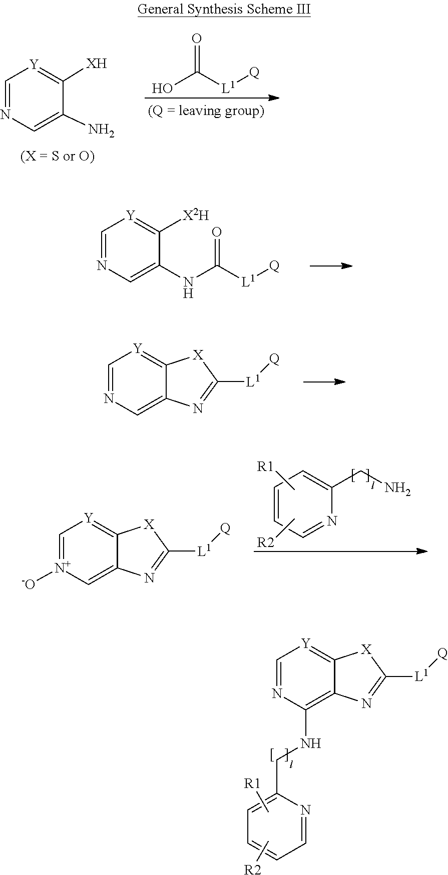

(wherein the leaving group Q may be OBn and wherein the position of “XH” and “NH₂” can be exchanged to prepare the so-called “iso-compounds” as described herein)

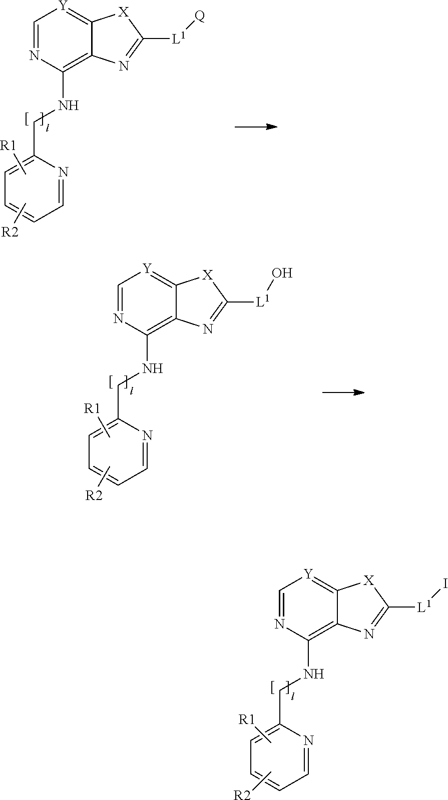

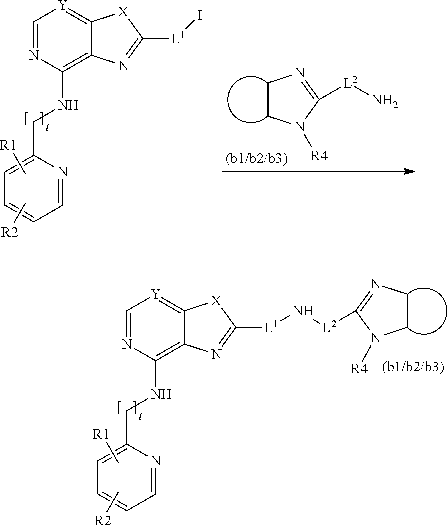

(wherein the structure indicated as “(b1/b2/b3)” represents the structures b-i, b-2 or b-3 as defined herein).

In particular, the following general procedures describe suitable preparation processes for preferred compounds of the invention:

### Synthesis of Intermediates

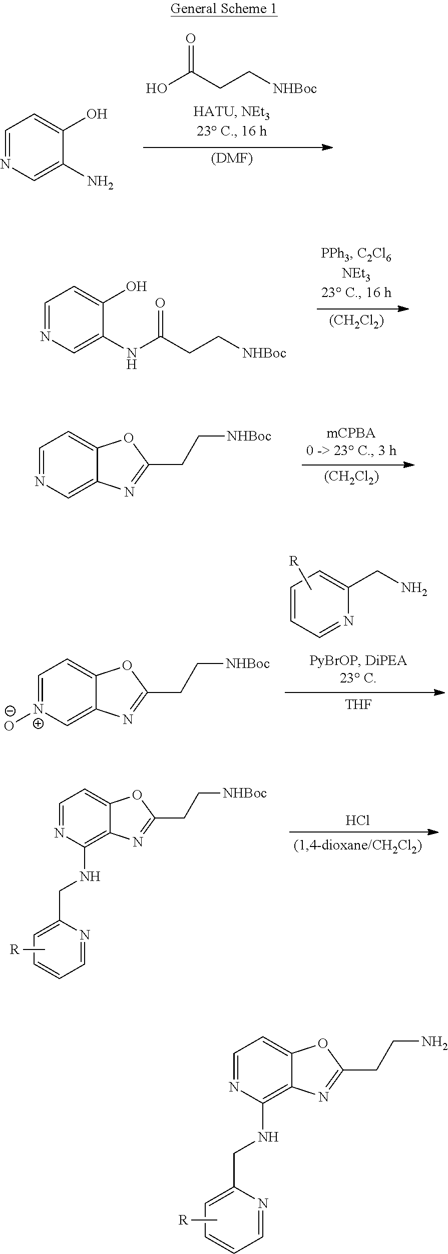

(wherein R indicates one substituent R¹ or R² as defined herein).

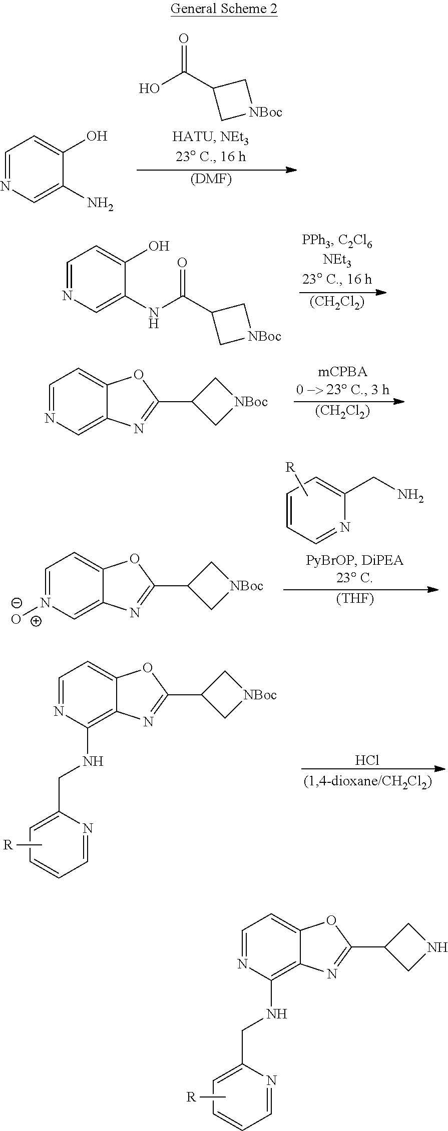

(wherein R indicates one substituent R¹ or R² as defined herein).

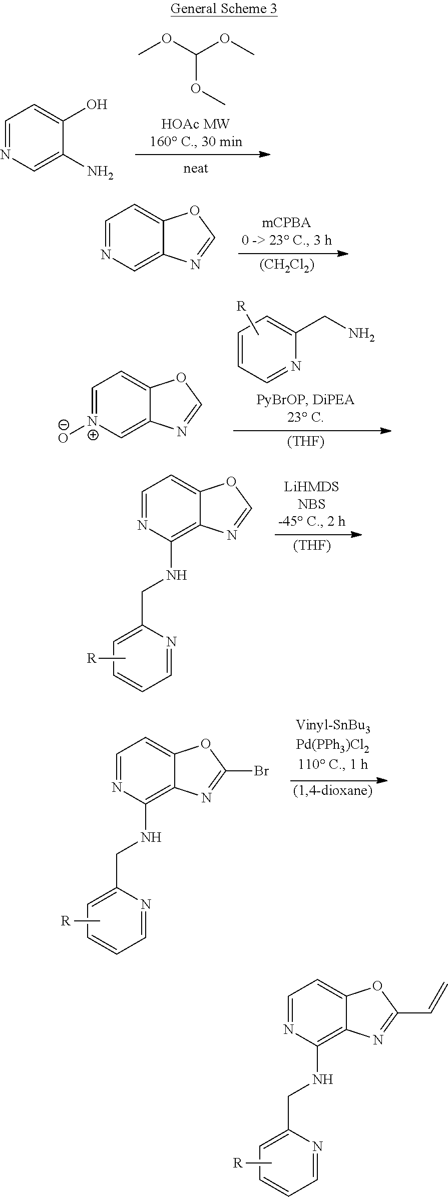

(wherein R indicates one substituent R¹ or R² as defined herein).

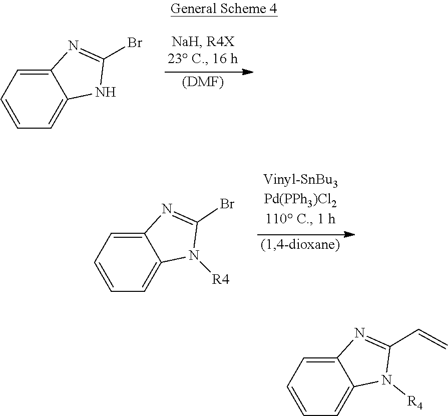

(wherein R⁴ may have the meaning as defined anywhere herein).

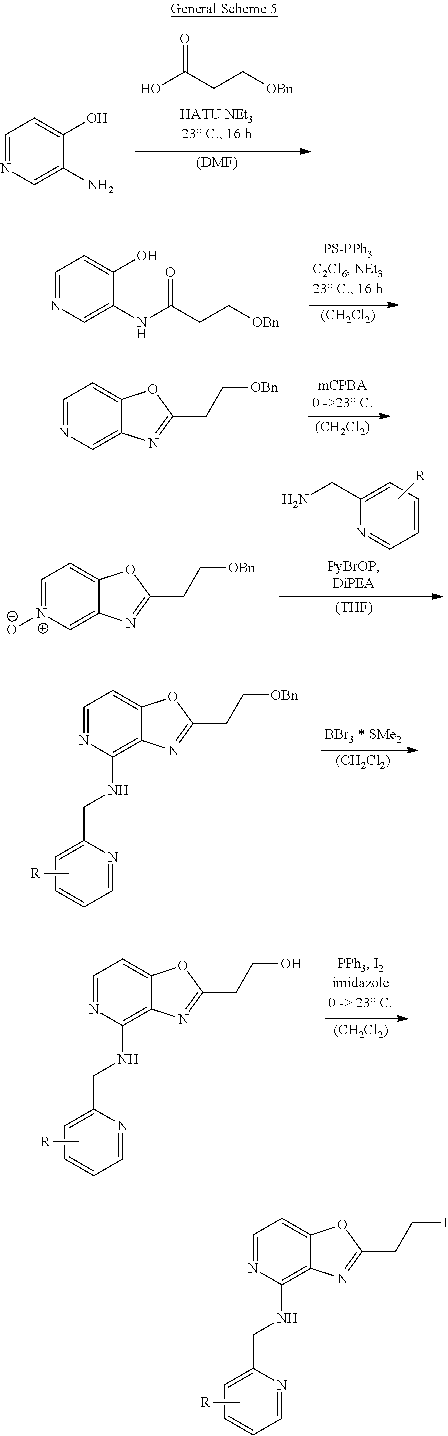

(wherein R indicates one substituent R¹ or R² as defined herein).

### Synthesis of Compounds of the Invention

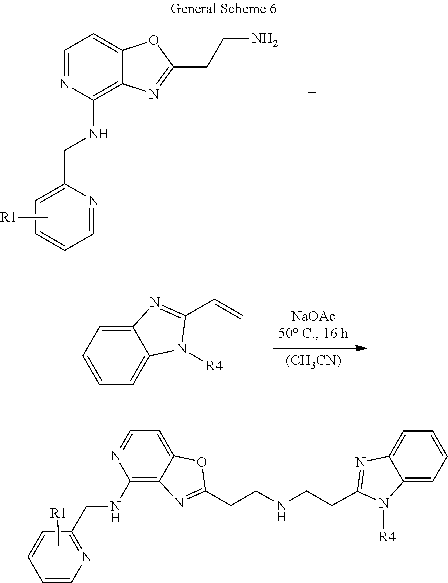

(wherein R¹ and R⁴ may have the meaning as defined anywhere herein).

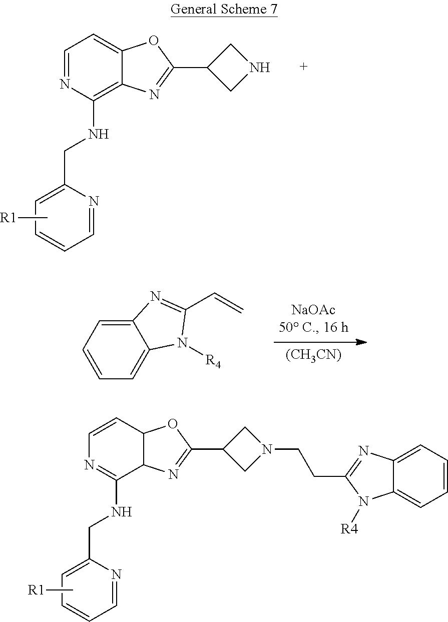

(wherein R¹ and R⁴ may have the meaning as defined anywhere herein).

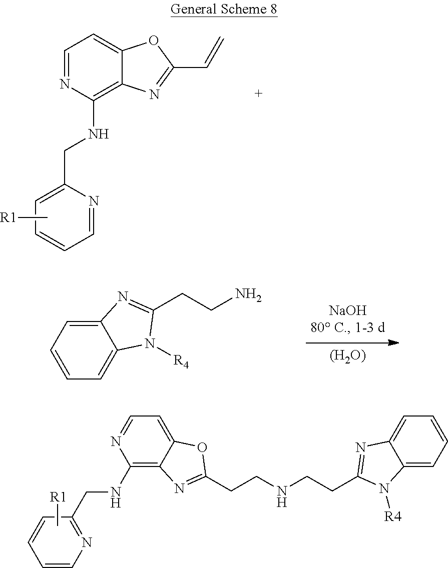

(wherein R¹ and R⁴ may have the meaning as defined anywhere herein).

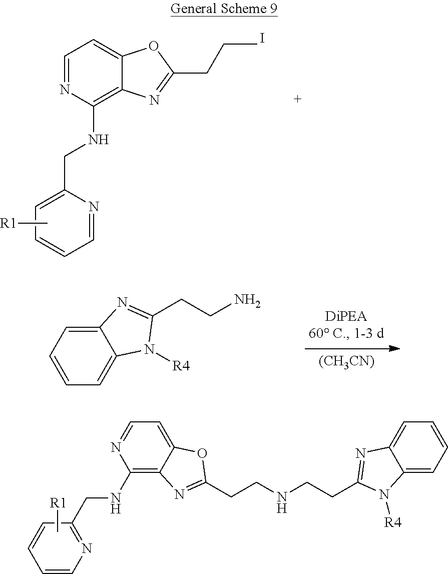

In a further aspect the invention covers the intermediate compounds obtainable in the preparation methods described herein, such as in particular the intermediate compounds resulting from the individual steps of the general reaction scheme above and as described further in detail herein. Details of the preparation conditions provide the Examples below.

### EXAMPLES

The invention is illustrated in more detail by the following examples. The examples are merely explanatory, and the person skilled in the art can extend the specific examples to further claimed compounds.

### Pharmacological Assays

### 1. Hepcidin Internalization Assay (J774)

This cellular assay allows quantification of the binding of hepcidin to ferroportin (Fpn) through microscopic detection of internalization of a fluorescently labeled hepcidin into J774 cells. J774 is a mouse macrophage cell line which was shown to express Fpn endogenously upon incubation with iron (Knutson et al, 2005). Binding of hepcidin to Fpn triggers internalization and degradation of both hepcidin and Fpn. However, the TMR (6-carboxytetramethylrhodamine) fluorophore attached to hepcidin remains associated with the cell after degradation of the hepcidin peptide backbone. Therefore, microscopic detection of cell-associated TMR fluorescence is a measure of hepcidin binding to Fpn and internalization of hepcidin and Fpn. If TMR-hepcidin is prevented from binding to Fpn, cellular TMR fluorescence remains low (Durrenberger et al, 2013). The effect of small molecular weight Fpn inhibitor compounds in this assay was evaluated in vitro as described below.

J774 cells, harvested from ca. 80% confluent cultures, were plated at 8×10⁵ cells/ml in complete medium (DMEM, 10% FBS, 1% Penicillin-Streptomycin) containing 200 μM Fe(III)NTA (nitrilotriacetic acid), 100 μl per well of 96 well MicroClear plates (Greiner; Cat. 655090) and grown at 37° C. with 5% CO₂. After overnight incubation, cells were washed 3 times with pre-warmed DMEM w/o phenol red, 30 μl/well of DMEM w/o phenol red was added after the final wash and 10 μl/well of dilution series of test compounds were added in triplicates. J774 cells were pre-incubated with test compounds at 37° C. with 5% CO₂ for 15 min. before TMR-hepcidin was added at 25 nM final concentration. Cells were incubated in a total volume of 50 μl at 37° C. with 5% CO₂ for 2 hours, then Hoechst 33342 dye was added to a final concentration of 0.5 μg/ml to stain nuclei and further incubated for 10 min. at 37° C. with 5% CO₂. Cells were washed 3 times with PBS and fixed in 100 μl of 4% paraformaldehyde in PBS for 15 min. at room temperature. After removal of the paraformaldehyde solution, cells were washed 3 times with PBS leaving 100 μl per well and the plates were sealed with foil plate seal. TMR (530-550 nm excitation/575-625 nm emission/400 ms exposure time) and Hoechst 33342 (360-370 nm excitation/420-460 nm emission/10 ms exposure time) fluorescence images were acquired using a ScanR plate imager (Olympus) with a 20× high NA objective. Four pictures were acquired per well and fluorescence channel covering ca. 1500 cells per well. The acquired image data was analysed with the ScanR image analysis software. Image analysis included detection of nuclei (Hoechst 33342 fluorescence), identification of cell-associated regions, application of a virtual channel and thresholding for rolling-ball-type background reduction, followed by application of the Sum(Mean) algorithm to measure the TMR fluorescence associated with cells as a quantitative measure for internalized TMR- hepcidin. IC₅₀ values were calculated with the Sum(Mean) raw data using “log(inhibitor) vs. response” curve fitting of Prism 5 software (GraphPad Software Inc., version 5.02). For each data set the fit of the “log(inhibitor) vs. response (three parameters)” model was compared to the fit of the “log(inhibitor) vs. response—Variable slope (four parameters)” model and the IC₅₀ data of the preferred model was used. IC₅₀ data of the Fpn inhibitors that were tested in the hepcidin internalization assay are listed in Table 1. The IC₅₀ of unlabeled hepcidin in this assay is 0.015±0.011 μM.

TABLE 1
Average (AVE) IC₅₀ data of Fpn inhibitors tested in the hepcidin internalization assay
is shown for multiple measurements

| | | IC50 |
|---|---|---|
| No. | Compound/Structure | [uM] |
| 1 | 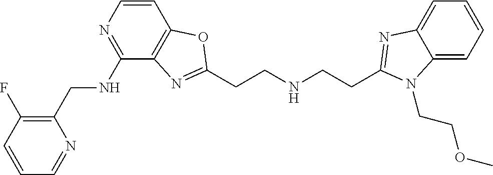 [SMILES: COCCn1c(CCNCCc2nc3c(NCc4ncccc4F)nccc3o2)nc2ccccc21] | 0.009 |
| 2 | 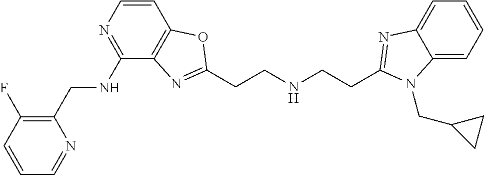 [SMILES: Fc1cccnc1CNc1nccc2oc(CCNCCc3nc4ccccc4n3CC3CC3)nc12] | 0.015 |
| 3 | 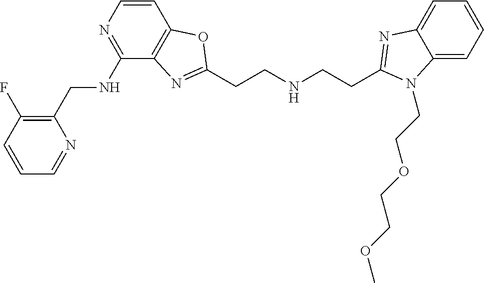 [SMILES: COCCOCCn1c(CCNCCc2nc3c(NCc4ncccc4F)nccc3o2)nc2ccccc21] | 0.009 |
| 4 | 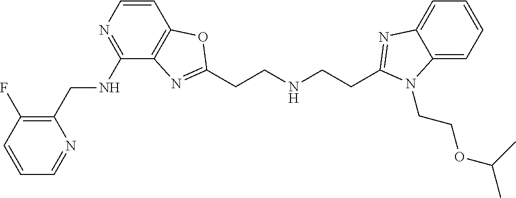 [SMILES: CC(C)OCCn1c(CCNCCc2nc3c(NCc4ncccc4F)nccc3o2)nc2ccccc21] | 0.004 |
| 5 | 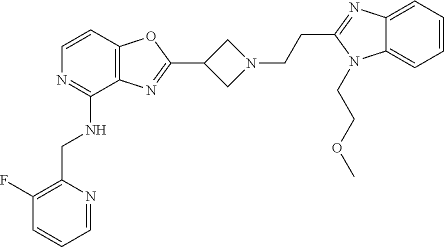 [SMILES: COCCn1c(CCN2CC(c3nc4c(NCc5ncccc5F)nccc4o3)C2)nc2ccccc21] | 0.007 |
| 6 | 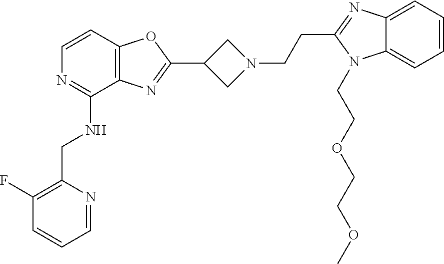 [SMILES: COCCOCCn1c(CCN2CC(c3nc4c(NCc5ncccc5F)nccc4o3)C2)nc2ccccc21] | 0.011 |
| 7 | 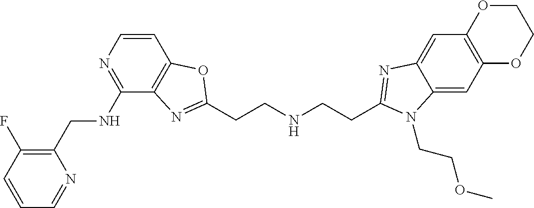 [SMILES: COCCn1c(CCNCCc2nc3c(NCc4ncccc4F)nccc3o2)nc2cc3c(cc21)OCCO3] | 0.083 |
| 8 | 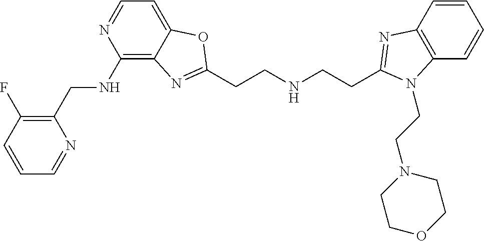 [SMILES: Fc1cccnc1CNc1nccc2oc(CCNCCc3nc4ccccc4n3CCN3CCOCC3)nc12] | 0.007 |
| 9 | 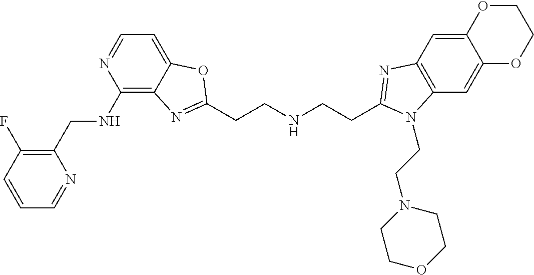 [SMILES: Fc1cccnc1CNc1nccc2oc(CCNCCc3nc4cc5c(cc4n3CCN3CCOCC3)OCCO5)nc12] | 0.827 |
| 10 | 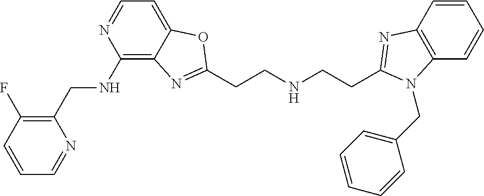 [SMILES: Fc1cccnc1CNc1nccc2oc(CCNCCc3nc4ccccc4n3Cc3ccccc3)nc12] | 0.008 |
| 11 | 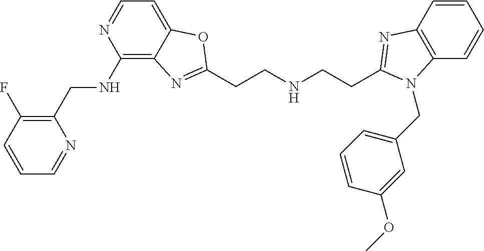 [SMILES: COc1cccc(Cn2c(CCNCCc3nc4c(NCc5ncccc5F)nccc4o3)nc3ccccc32)c1] | 0.005 |
| 12 | 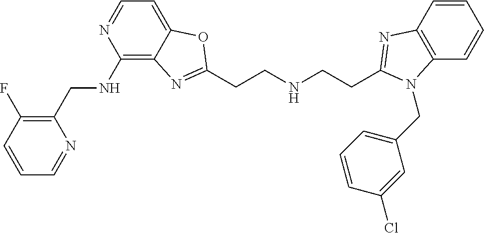 [SMILES: Fc1cccnc1CNc1nccc2oc(CCNCCc3nc4ccccc4n3Cc3cccc(Cl)c3)nc12] | 0.028 |
| 13 | 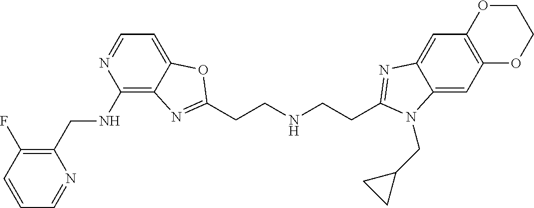 [SMILES: Fc1cccnc1CNc1nccc2oc(CCNCCc3nc4cc5c(cc4n3CC3CC3)OCCO5)nc12] | 0.005 |
| 14 | 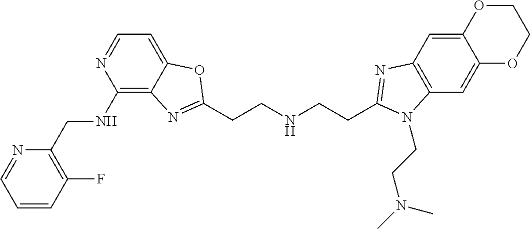 [SMILES: CN(C)CCn1c(CCNCCc2nc3c(NCc4ncccc4F)nccc3o2)nc2cc3c(cc21)OCCO3] | 0.81 |
| 15 | 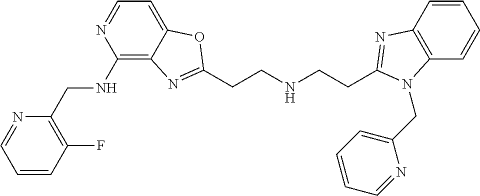 [SMILES: Fc1cccnc1CNc1nccc2oc(CCNCCc3nc4ccccc4n3Cc3ccccn3)nc12] | 9.50 |
| 16 | 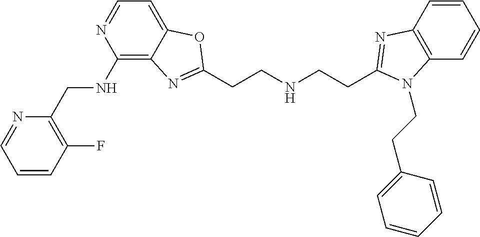 [SMILES: Fc1cccnc1CNc1nccc2oc(CCNCCc3nc4ccccc4n3CCc3ccccc3)nc12] | 40.2 |
| 17 | 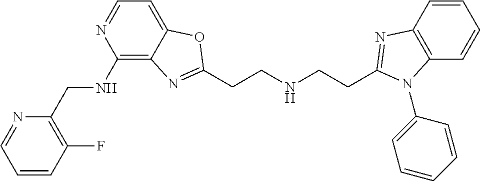 [SMILES: Fc1cccnc1CNc1nccc2oc(CCNCCc3nc4ccccc4n3-c3ccccc3)nc12] | 0.025 |
| 18 | 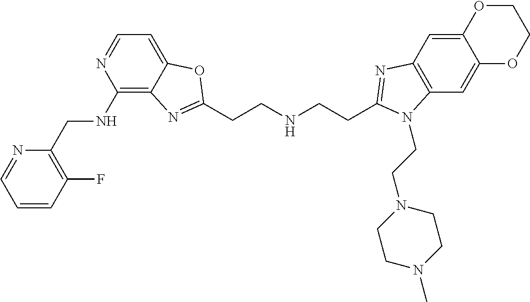 [SMILES: CN1CCN(CCn2c(CCNCCc3nc4c(NCc5ncccc5F)nccc4o3)nc3cc4c(cc32)OCCO4)CC1] | 20.0 |
| 19 | 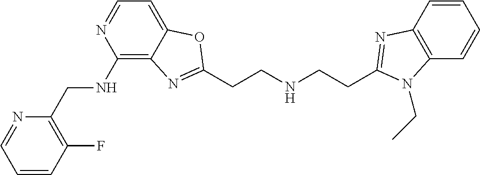 [SMILES: CCn1c(CCNCCc2nc3c(NCc4ncccc4F)nccc3o2)nc2ccccc21] | 0.018 |
| 20 | 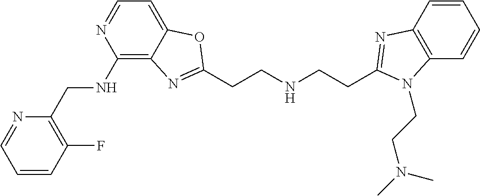 [SMILES: CN(C)CCn1c(CCNCCc2nc3c(NCc4ncccc4F)nccc3o2)nc2ccccc21] | 20.0 |
| 21 | 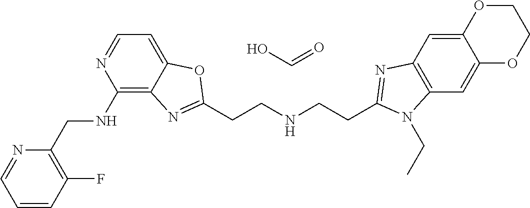 | 0.031 |
| 22 | 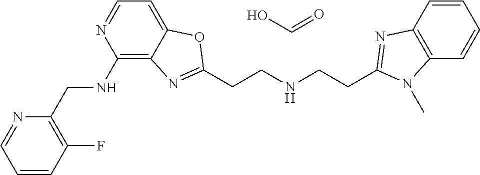 | 0.040 |
| 23 | 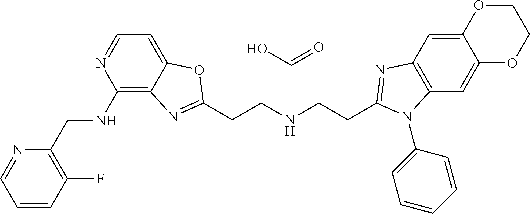 | 0.018 |

### 2. Biophysical Ferroportin-Hepcidin Binding Assay

This biophysical assay was developed to confirm inhibition of hepcidin binding to ferroportin (Fpn) more directly. Incubation of TMR-hepcidin with purified human Fpn isolated from Pichia pastoris yeast cells expressing human Fpn with a C-terminal FLAG affinity tag (Bonaccorsi di Patti, 2014) leads to increased fluorescence polarization (FP) of the TMR-hepcidin ligand. Small molecular weight Fpn inhibitors are tested for inhibition of binding of TMR-hepcidin to Fpn, as detected by dose-dependent decrease of the TMR FP signal, as described in detail below.

A mixture of 1.3 μM human Fpn and 30 nM TMR-hepcidin in FP assay buffer containing 50 mM Tris-HCl pH 7.3, 200 mM NaCl, 0.02% DDM, 0.1% BSA is plated into a 384 well black low volume round bottom plate (Corning, Cat. 3677) at 16 μl per well. 8 μl of serial dilutions of test compounds are added in duplicates to reach final Fpn and TMR-hepcidin concentrations of 1 M and 20 nM, respectively. Plates are incubated for 90 minutes at room temperature and parallel (S) and perpendicular (P) fluorescence is measured in a Synergy H1 fluorescence reader (BioTek). FP values are calculated in mP according to the following formula.

$$mP = \frac{{F}_{p a rallel} - {F}_{perpendicular}}{{F}_{parallel} + {F}_{perpendicular}}$$

IC₅₀ values are determined with the calculated mP values as described for the hepcidin internalization assay. The IC₅₀ of unlabeled hepcidin in this assay is about 0.37±0.067 μM.

### 3. Inhibition of Ferroportin Mediated Iron Export Activity in an Iron Response Assay

Intracellular iron levels are indirectly measured in this assay by monitoring the activity of a beta-liactamase (BLA) reporter ene fused to the human ferritin promoter and the associated iron regulatory element (IRE) contained within the 5′ untranslated region of the ferritin mRNA. Expression of ferroportin (Fpn) in such a cell line leads to iron efflux and lower iron levels as reflected by lower activity of the reporter gene. On the other hand, inhibition of Fpn-mediated iron efflux results in elevated cellular iron levels which is detected as increased reporter gene activity. Small molecular weight Fpn inhibitor compounds are tested for dose-dependent effects in this in vitro iron response assay as described below.

The HEK-293 cell line #354 is generated by stable integration of (i) a human Fpn-GFP fusion construct inserted in a derivative of the doxycycline-inducible pTRE-Tight-BI plasmid (Clontech, Cat. 631068) and (ii) a human ferritin promoter-BLA reporter gene into a derivative of the HEK-293 Tet-ON Advanced cell line (Clontech). To generate the ferritin-BLA reporter gene construct, a 1.4 kb fragment of the human ferritin H promoter is amplified by PCR from human genomic DNA (forward primer 5′-CAGGTTTGTGAGCATCCTGAA-3′; reverse primer 5′-GGCGGCGACTAAGGAGAGG-3′) and inserted in front of the BLA gene present in the pcDNA™6.2/cGeneBLAzer™-DEST plasmid (Invitrogen, Cat. 12578-043) thereby replacing the original CMV promoter and placing the IRE that regulates translation of the ferritin gene ca. 170 bp upstream of the start codon of the reporter gene. #354 cells are harvested from ca. 80% confluent cultures, seeded at 1.8×10⁵ cells/ml in DMEM/F12 GlutaMAX™ medium (Invitrogen, Cat. 31331-028) containing 10% FBS (Clontech, Cat. 631106), 1% Penicillin-Streptomycin, 200 μg/ml Hygromycin B (Invitrogen, Cat. 10687-010), Blasticidin 5 μg/ml, (Invitrogen, Cat. R210-01), 4 μg/ml doxycycline (Clontech, Cat. 631311), 50 μl per well of 384 well PDL-coated plates and grown at 37° C. with 5% CO₂. After overnight incubation, 10 μl/well of dilution series of the test compounds are added in quadruplicates and plates are further incubated overnight at 37° C. with 5% CO₂. Cells are washed 3 times with HBSS leaving 25 μl per well. BLA activity is detected by adding 5 μl/well of the GeneBlazer reagent CCF4-AM (Invitrogen, Cat. K1085) to the cells. After incubation of the plates in the dark at 18° C. for 60 min., blue and green fluorescence signals are measured in a Safire2 fluorescence plate reader (Tecan) with excitation at 410 nm and emissions at 458 nm (blue) and 522 nm (green). The ratio of blue/green fluorescence as a measure for BLA activity is calculated and EC₅₀ values are determined with the calculated blue/green fluorescence ratios as described for the hepcidin internalization assay. The EC₅₀ of hepcidin in this assay is about 0.096±0.063 μM (n=37).

### 4. Ferroportin Internalization and Degradation Assay

HEK-293 cell line #354 (described in example 3) is used to measure the capacity of the compounds to induce internalization and degradation of ferroportin (Fpn) by fluorescence activated cell sorting (FACS). Growing HEK-293 #354 cells in doxycycline containing media induced expression of human Fpn-GFP fusion protein on the cell surface. Data from 10 independent experiments show that cultivation of HEK #354 cells for 48 h in the presence of 4 μg/ml doxycycline induce in average 42.6%±6.4% Fpn-GFP-positive cells. Small molecular weight Fpn inhibitor compounds are tested for dose-dependent effects on the Fpn-GFP mean fluorescence intensity (MFI) on HEK-293 cell line #354, as described below.

HEK #354 cells are harvested from ca. 80% confluent cultures, seeded at 0.6×10⁶ cells/ml in DMEM/F12 GlutaMAX™ medium (Invitrogen, Cat. 31331-028) containing 10% FBS (Clontech, Cat. 631106), 1% Penicillin-Streptomycin (Invitrogen, Cat. 15140-122), 200 μg/ml Hygromycin B (Invitrogen, Cat. 10687-010), Blasticidin 5 μg/ml, (Invitrogen, Cat. R210-01), 4 μg/ml doxycycline (Clontech, Cat. 631311), 50 μl per well of 384 well plates (Greiner; Cat. 781091) and grown at 37° C. with 5% CO₂. After overnight incubation, 10 μl/well of dilution series of the test compounds are added in quadruplicates and plates are further incubated overnight at 37° C. with 5% CO₂. Cells are washed once with FACS buffer (PBS containing 1% FBS, 2 mM EDTA and 0.05% NaN₃), harvested in FACS buffer with 0.5 μg/ml propidium iodide (Sigma, Cat. P4864) and analyzed in a flow cytometer (CANTO™ II, BD Biosciences) equipped with high throughput sampler. Live HEK #354 cells are gated as propidium iodide negative population and analyzed for expression of Fpn-GFP. MFI of Fpn-GFP of >2000 live cells for each compound dilution is calculated using FlowJo (Tree Star's, Oregon) and the potency of the Fpn-inhibitors to induce internalization and degradation of Fpn-GFP is calculated as described for the hepcidin internalization assay. The average EC₅₀ value of hepcidin in this assay is about 0.004±0.002 μM.

### 5. Ferroportin Ubiquitination and Degradation

Exposure of cells expressing ferroportin (Fpn) to hepcidin is known to trigger ubiquitination and subsequent internalization and degradation of Fpn (Qiao, 2012). The potential of Fpn inhibitors to induce Fpn ubiquitination and degradation is investigated with an immunoprecipitation assay using the J774 mouse macrophage cell line which expresses Fpn upon treatment with iron.

J774 cells (DSMZ, Cat. ACC170) are seeded at 0.8×106 cells/ml in 15 ml of medium (DMEM Gibco Cat. 11971-025, 10% heat inactivated FBS Gibco Cat. 10500-064, 1% Penicillin-Streptomycin Gibco Cat. 15140-122) containing 200 μM Fe(III)-NTA into 10 cm tissue culture dishes (Greiner Cat. 664160) and grown overnight at 37° C. with 5% CO₂.

Cells are incubated with synthetic human hepcidin (Bachem, Cat. H-5926) or Fpn inhibitor compounds for 10 min or 120 min. Cells are washed and lysed with ice-cold lysis buffer (Pierce, Life Technologies, Cat. 87787) including 1× HALT protease inhibitor cocktail (Life technologies, Cat. 78429) and 10 mM iodoacetamide (Sigma, Cat. 16125) to stabilize ubiquitinated proteins. Immunoprecipitation is done using the Pierce Classic IP Kit (Life Technologies, Cat. 26146) following the manufacturer's protocol. Briefly, 2 mg protein in 1.25 ml IP lysis buffer is incubated by mixing for 1 h at 4° C. with control agarose beads to pre-clear the lysate and reduce nonspecific signal. Unbound lysate is then incubated overnight with 12 μg per reaction of the affinity purified anti-Fpn antibody F308 that is raised against a GST fusion protein of mouse Fpn amino acids 224-308. Immune complexes are captured by pipetting 14 μl settled Pierce Protein A/G Plus Agarose beads (Life Technologies, Cat. 20423) per reaction and the slurry is incubated for 1.5 h at 4° C. with gentle end-over-end mixing. The beads are washed and immune complexes are eluted directly with 75 μl SDS NuPAGE LDS sample buffer (Life Technologies, Cat. NP0007) containing DTT (Life Technologies, Cat. NP0009).

After immunoprecipitation samples are analyzed by Western blotting using a rabbit anti-mouse MTP1 antiserum (Alpha Diagnostic International, Cat. MTP11-A) and a mouse anti-mono- and polyubiquitinylated conjugates monoclonal antibody (Enzo Lifesciences, Cat. BML-PW8810) for detection of ferroportin and ubiquitin, respectively. Mouse monoclonal anti-rabbit IgG light chain (Abcam, Cat. ab99697) and anti-mouse IgG H&L (Abcam, Cat. ab6789) HRP conjugates are used as secondary antibodies.

### 6. Inhibition of Iron Efflux by Ferroportin Inhibitors

The activity of hepcidin and ferroportin inhibitor compounds regarding their ability to block iron export via ferroportin is tested on T47D cells (ECACC, Cat. 85102201) as described below.

Cells are plated in 24-well plates (Greiner, Cat. 662160) containing 350,000 cells/well and incubated overnight with 100 μM ⁵⁸Fe (⁵⁸Fe(II)-Sulfate, Vifor Pharma Batch No. ROR 3085) in 500 μM L-Ascorbic Acid (Sigma Aldrich, Cat. 795437) containing growth medium. Cells were washed once with 500 μl iron uptake buffer (IUB; PIPES 40 mM, Cat. P1851, Glucose Monohydrate 10 mM, Cat. 49158, Sodium Chloride 260 mM, Cat. 71379, Potassium Chloride 20 mM, Cat. P9541, Magnesium Sulfate 2 mM, Cat. 63138, Sigma Aldrich), then once with removal buffer (2 min incubation, BPDS 100 μM, Cat. 11890 and Na₂S₂O₄ 500 μM, Cat. 157953, Sigma Aldrich, in IUB) and again twice with IUB. A serial dilution of hepdicin (Bachem) or ferroportin inhibitors (4 μM-0.0064 μM, 5 fold dilution) is added in a total volume of 0.6 ml per well. Cells are incubated at 37° C. with 5% CO2 for 20 h. Supernatants are collected and ⁵⁸Fe is measured using inductively coupled plasma mass spectrometry (ICP-MS, Thermo Scientific, Element 2). Pellets are harvested for protein concentration measurements. Results are plotted as ng ⁵⁸Fe in supernatant per mg protein in cell lysates.

### Preparation of Example Compounds

### General Experimental Details

Commercially available reagents and solvents (HPLC grade) were used without further purification. ¹H NMR spectra were recorded on a Bruker DRX 500 MHz spectrometer, a Bruker DPX 250 MHz spectrometer or a Bruker Avance spectrometer 400 MHz in deuterated solvents. Chemical shifts (6) are in parts per million.

Compounds were purified by flash column chromatography on normal phase silica on Biotage Isolera systems using the appropriate SNAP cartridge and gradient. Alternatively, compounds were purified on reverse phase using Biotage Isolera systems with the appropriate C18 SNAP cartridge and reverse-phase eluent or by preparative HPLC (if stated otherwise).

### Abbreviations

EtOAc Ethylacetate
CH₂Cl₂ Dichloromethane
Et₂O Diethylether
MeOH Methanol
EtOH Ethanol
brine Aqueous saturated sodium chloride solution
Chloroform-d Deuterated chloroform
DMSO-d₆ Deuterated dimethylsulfoxid
s Singlet
br s Bright Singlet
d Doublet
dd Double Doublets
dt Doublet of Triplets
td Triplet of Doublets
hept. Heptett
m Multiplet
q Quartet
δ Chemical shift
ppm Parts per million
M Molarity
mm Millimolar
umol Mikromolar
g Gram
mg Milligram
l Liter
mL Milliliter
h Hours
min Minute
%-w/w Percentage by mass
TLC Thin layer chromatography
UHPLC Ultra high pressure liquid chromatography
MS Mass spectroscopy
ESI Electronic spray ionization
m/z mass to charge ratio
H⁺ Proton
MHz Mega Hertz
s.m. starting material
Jones Reagent CrO₃ in H₂SO₄
CrO₃ Chromium trioxide
HCl Hydrochloric acid
H₂SO₄ Sulfuric acid
NH₄Cl Ammonium chloride
Na₂SO₄ Sodium sulfate
NaOH Sodium hydroxide
Bn Benzyl
MS Mass spectra
ESI Electrospray ionisation
SNAP Biotage-column-brand name for flash column chromatography
R_{f} Retention Factor
TLC Thin Layer Chromatography

### Chemical Nomenclature

The chemical names of the intermediates and the final Example Compounds were generated by using Chem Draw Professional 17.0.

All R_{f} values were determined using the following TLC plates: Merck, TLC Silcagel 60 F₂₅₄.

### Preparation Details

### Intermediates

### A. Tert-butyl (3-((4-hydroxypyridin-3-yl)amino)-3-oxopropyl)carbamate

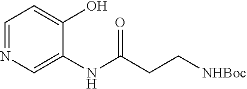 [SMILES: CC(C)(C)OC(=O)NCCC(=O)Nc1cnccc1O]

To a solution of 3-aminopyridin-4-ol (5.00 g, 44.0 mmol, 1 eq.) in N,N-dimethylformamid (90 mL) was added 3-((tert-butoxycarbonyl)amino)propanoic acid (8.42 g, 44.0 mmol, 1 eq.), triethylamine (12.4 mL, 88.1 mmol) and HATU (20.7 g, 52.9 mmol, 1.2 eq.). The reaction mixture was stirred at 23° C. for 16 hours. The solution was diluted with water and extracted with ethyl acetate (3×). The combined organic phase was washed with water and brine, dried over sodium sulfate, filtered and concentrated under reduced pressure. The crude oil was purified by flash column chromatography (heptanes/EtOAc, 0-100% EtOAc) to give the title tert-butyl (3-((4-hydroxypyridin-3-yl)amino)-3-oxopropyl)carbamate (11.7 g, 41.6 mmol, 94%) as a white foam.

LCMS (ESI) m/z=282.2. 1H NMR (400 MHz, DMSO) δ 11.47 (s, 1H), 9.02 (s, 1H), 8.83-8.59 (m, 1H), 7.63 (dd, J=7.1, 1.6 Hz, 1H), 6.76 (t, J=5.7 Hz, 1H), 6.23 (d, J=7.1 Hz, 1H), 3.22-3.13 (m, 3H), 2.57-2.49 (m, 2H), 1.35 (s, 9H) ppm.

### B. Tert-butyl (2-(oxazolo[4,5-c]pyridin-2-yl)ethyl)carbamate

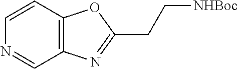 [SMILES: CC(C)(C)OC(=O)NCCc1nc2cnccc2o1]

To a suspension of polymer supported triphenyl phosphine (loading 1.6 mmol/g, 7.75 g, 12.4 mmol, 2 eq.) in dichloromethane (60 mL) was added hexachloroethane (1.83 g, 7.73 mmol, 1.25 eq.) and triethylamine (4.31 mL, 30.9 mmol, 5 eq.). The suspension was stirred for 5 min, before tert-butyl (3-((4-hydroxypyridin-3-yl)amino)-3-oxopropyl)carbamate (1.74 g, 6.19 mmol, 1.0 eq.) was added. The mixture was stirred 1 h at r.t. Another portion of 1,1,1,2,2,2-hexachloroethane (1.10 g) and triethylamine (1.42 mL) was added and stirring was continued at 23° C. for 16 hours. The reaction mixture was diluted with dichloromethane and acetonitrile and then filtered. The filtrate was concentrated and the resulting crude oil purified by flash column chromatography (Heptanes/EtOAc, 0-100% EtOAc) to obtain the desired tert-butyl (2-(oxazolo[4,5-c]pyridin-2-yl)ethyl)carbamate (1.12 g, 4.25 mmol, 69%).

LCMS (ESI) m/z=264.1. 1H NMR (400 MHz, DMSO) δ 8.97 (s, 1H), 8.51 (d, J=5.5 Hz, 1H), 7.02 (t, J=6.0 Hz, 1H), 3.41 (q, J=6.5 Hz, 2H), 3.07 (t, J=6.7 Hz, 2H), 1.30 (s, 9H) ppm.

### C. 2-(2-((tert-butoxycarbonyl)amino)ethyl)oxazolo[4,5-c]pyridine 5-oxide

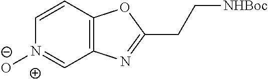

tert-butyl (2-(oxazolo[4,5-c]pyridin-2-yl)ethyl)carbamate (3.04 g, 11.5 mmol, 1 eq.) was dissolved in dichloromethane (100 mL). The solution was cooled to 0° C., before mCPBA (75% Wt, 5.31 g, 23.1 mmol, 2 eq.) was added. After 2 h the ice-bath was removed and stirring was continued at 23° C. until LCMS indicated full conversion of s.m. The reaction mixture was diluted with dichloromethane and washed with aq. sat. sodium bicarbonate solution and brine, dried over sodium sulfate, filtered and concentrated under reduced pressure. The crude material was suspended in methanol. The solid was filtered-off, washed with methanol and dried under vacuum to yield the desired 2-(2-((tert-butoxycarbonyl)amino)ethyl)oxazolo[4,5-c]pyridine 5-oxide (1.78 g, 6.37 mmol, 55%) as an off-white solid.

LCMS (ESI) m/z=280.3. 1H NMR (400 MHz, DMSO) δ 8.77 (d, J=1.8 Hz, 1H), 8.20 (dd, J=7.0, 1.8 Hz, 1H), 7.80 (d, J=7.1 Hz, 1H), 7.02 (t, J=5.9 Hz, 1H), 3.39 (q, J=6.4 Hz, 2H), 3.04 (t, J=6.6 Hz, 2H), 1.31 (s, 9H) ppm.

### D. Tert-butyl (2-(4-(((3-fluoropyridin-2-yl)methyl)amino)oxazolo[4,5-c]pyridin-2-yl)ethyl)carbamate

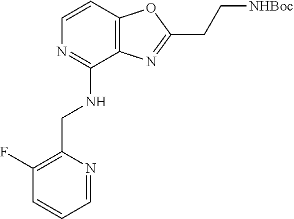 [SMILES: CC(C)(C)OC(=O)NCCc1nc2c(NCc3ncccc3F)nccc2o1]

To a solution of 2-(2-((tert-butoxycarbonyl)amino)ethyl)oxazolo[4,5-c]pyridine 5-oxide (1.78 g, 6.37 mmol, 1 eq.) and (3-fluoropyridin-2-yl)methanamine dihydrochloride (1.90 g, 9.56 mmol, 1.5 eq.) in dry tetrahydrofuran (60 mL) was added DIPEA (4.12 g, 5.55 mL, 31.9 mmol, 5 eq.) and bromotri(pyrrolidin-1-yl)phosphonium (I2-fluoraneyl)pentafluorophosphate(V) (4.47 g, 9.56 mmol, 1.5 eq.). The reaction mixture was stirred at 23° C. until LC/MS indicated full consumption of the s.m. The reaction mixture was diluted with ethyl acetate and washed with aq. sat. ammoniumchloride solution, aq. sat. sodium bicarbonate solution and brine. The organic phase was dried over sodium sulfate, filtered and concentrated under reduced pressure. The crude material was purified by flash column chromatography (CH₂Cl₂/MeOH/NH₃ 90/9/1) to yield the desired tert-butyl (2-(4-(((3-fluoropyridin-2-yl)methyl)amino)oxazolo[4,5-c]pyridin-2-yl)ethyl)carbamate (2.31 g, 5.96 mmol, 94%).

LCMS (ESI) m/z=388.6. 1H NMR (400 MHz, DMSO) δ 8.31 (dd, J=4.9, 1.8 Hz, 1H), 7.92-7.84 (m, 1H), 7.73 (ddd, J=10.0, 8.3, 1.3 Hz, 1H), 7.42 (dt, J=8.6, 4.5 Hz, 1H), 7.28 (d, J=6.6 Hz, 1H), 7.00 (t, J=5.9 Hz, 1H), 4.97 (s, 1H), 3.41-3.36 (m, 2H), 3.06 (t, J=6.6 Hz, 2H), 1.29 (s, 9H) ppm.

### E. 2-(2-aminoethyl)-N-((3-fluoropyridin-2-yl)methyl)oxazolo[4,5-c]pyridin-4-amine

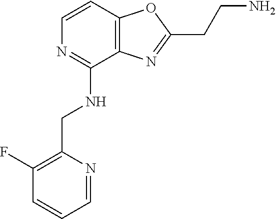 [SMILES: NCCc1nc2c(NCc3ncccc3F)nccc2o1]

tert-butyl (2-(4-(((3-fluoropyridin-2-yl)methyl)amino)oxazolo[4,5-c]pyridin-2-yl)ethyl) carbamate (2.31 g, 5.96 mmol, 1 eq.) was dissolved in dichloromethane (60 mL) and then treated with HCl in dioxane (4N, 14.9 mL, 59.6 mmol, 10 eq.). The reaction mixture was neutralized with 7N Ammonia in methanol and then concentrated. The crude mixture was purified by flash column chromatography (CH₂Cl₂/1N NH₃ in MeOH, 90/10) to afford the desired 2-(2-aminoethyl)-N-((3-fluoropyridin-2-yl)methyl)oxazolo[4,5-c]pyridin-4-amine (1.45 g, 5.05 mmol, 85%) as a pale yellow oil.

LCMS (ESI) m/z=288.2. 1H NMR (400 MHz, DMSO) δ 8.35 (dt, J=4.7, 1.5 Hz, 1H), 7.85 (d, J=5.7 Hz, 1H), 7.67 (ddd, J=10.3, 8.3, 1.3 Hz, 1H), 7.37 (dt, J=8.5, 4.4 Hz, 1H), 7.18 (t, J=5.6 Hz, 1H), 6.91 (d, J=5.7 Hz, 1H), 4.83 (dd, J=5.6, 1.8 Hz, 2H), 3.07-2.91 (m, 4H) ppm.

### F. tert-butyl 3-((4-hydroxypyridin-3-yl)carbamoyl)azetidine-1-carboxylate

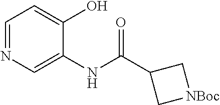 [SMILES: CC(C)(C)OC(=O)N1CC(C(=O)Nc2cnccc2O)C1]

To a solution of 3-aminopyridin-4-ol (3.00 g, 27.2 mmol, 1 eq.) in DMF (50 mL) was added 1-(tert-butoxycarbonyl)azetidine-3-carboxylic acid (5.48 g, 27.2 mmol, 1 eq.), triethylamine (7.59 mL, 54.5 mmol, 2 eq.) and HATU (12.4 g, 1.2 Eq, 32.7 mmol). The reaction mixture was stirred at 23° C. for 16 hours. The solution was diluted with water and extracted with ethyl acetate (3×). The combined organic phase was washed with water and brine, dried over sodium sulfate, filtered and concentrated under reduced pressure.

The crude oil was purified by flash column chromatography (CH₂Cl₂/MeOH, 0-15% MeOH) to give the desired tert-butyl 3-((4-hydroxypyridin-3-yl)carbamoyl)-azetidine-1-carboxylate (1.82 g, 6.20 mmol, 22.8%) as a white foam.

LCMS (ESI) m/z=288.2 [M-tBuOH]. 1H NMR (400 MHz, DMSO) δ 11.45 (s, 1H), 9.25 (s, 1H), 8.71 (d, J=3.5 Hz, 1H), 7.63 (d, J=7.1 Hz, 1H), 6.22 (d, J=7.1 Hz, 1H), 3.99-3.84 (m, 4H), 3.75 (tt, J=8.7, 5.7 Hz, 1H), 1.37 (s, 9H) ppm.

### G. tert-butyl 3-(oxazolo[4,5-c]pyridin-2-yl)azetidine-1-carboxylate

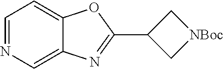 [SMILES: CC(C)(C)OC(=O)N1CC(c2nc3cnccc3o2)C1]

To a polymer supported triphenyl phosphine (loading 1.6 mmol/g, 7.75 g, 12.4 mmol, 2 eq.) in dichloromethane (60 mL) was added Hexachloroethane (1.84 g, 7.76 mmol, 1.25 eq.) and triethylamine (4.32 mL, 31.0 mmol, 5 eq.). The suspension was stirred for 5 min, before solid tert-butyl (3-((4-hydroxypyridin-3-yl)amino)-3-oxopropyl)carbamate (1.82 g, 6.20 mmol, 1 eq.) was added. The mixture was stirred 1 h at r.t. Another portion of 1,1,1,2,2,2-hexachloroethane (1.10 g) and triethylamine (1.42 mL) was added and stirring was continued at 23° C. for 16 hours. The reaction mixture was diluted with dichloromethane and acetonitrile and then filtered. The filtrate was concentrated and then purified by flash column chromatography (heptanes/EtOAc, 0-100% EtOAc) to obtain the desired tert-butyl 3-(oxazolo[4,5-c]pyridin-2-yl)azetidine-1-carboxylate (1.28 g, 4.65 mmol, 75%).

LCMS (ESI) m/z=276.2. 1H NMR (400 MHz, DMSO) δ 9.03 (d, J=1.0 Hz, 1H), 8.55 (d, J=5.6 Hz, 1H), 7.82 (dd, J=5.5, 1.0 Hz, 1H), 4.28 (d, J=8.4 Hz, 2H), 4.23-4.14 (m, 3H), 3.09 (qd, J=7.3, 3.3 Hz, 1H), 1.39 (s, 9H) ppm.

### H. 2-(1-(tert-butoxycarbonyl)azetidin-3-yl)oxazolo[4,5-c]pyridine 5-oxide

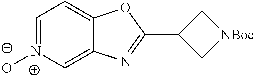 [SMILES: CC(C)(C)OC(=O)N1CC(c2nc3c[n+]([O-])ccc3o2)C1]

Tert-butyl 3-(oxazolo[4,5-c]pyridin-2-yl)azetidine-1-carboxylate (1.28 g, 4.65 mmol, 1 eq.) was dissolved in dichloromethane (45 mL). The solution was cooled to 0° C., before mCPBA (75% Wt, 2.14 g, 9.30 mmol, 2 eq.) was added. After 2 h the ice-bath was removed and stirring was continued at 23° C. until LCMS indicated full conversion of s.m. The reaction mixture was diluted with dichloromethane and washed with aq. sat. sodium bicarbonate solution and brine, dried over sodium sulfate, filtered and concentrated under reduced pressure. The crude material was suspended in methanol. The solid was filtered-off, washed with methanol and dried under vacuum to yield the desired 2-(1-(tert-butoxycarbonyl)-azetidin-3-yl)oxazolo[4,5-c]pyridine 5-oxide (716 mg, 2.46 mmol, 53%) as an off-white solid.

LCMS (ESI) m/z=292.2. 1H NMR (400 MHz, DMSO) δ 8.83 (dd, J=1.8, 0.6 Hz, 1H), 8.23 (dd, J=7.1, 1.8 Hz, 1H), 7.85 (dd, J=7.1, 0.7 Hz, 1H), 4.31-4.22 (m, 2H), 4.20-4.09 (m, 3H), 1.39 (s, 9H) ppm.

### I. Tert-butyl 3-(4-(((3-fluoropyridin-2-yl)methyl)amino)oxazolo[4,5-c]pyridin-2-yl)azetidine-1-carboxylate

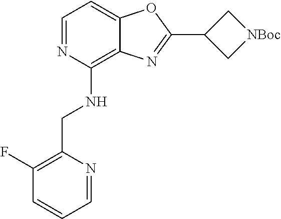 [SMILES: CC(C)(C)OC(=O)N1CC(c2nc3c(NCc4ncccc4F)nccc3o2)C1]

To a solution of 2-(1-(tert-butoxycarbonyl)azetidin-3-yl)oxazolo[4,5-c]pyridine 5-oxide (716 mg, 2.46 mmol, 1 eq.) and (3-fluoropyridin-2-yl)methanamine dihydrochloride (734 mg, 3.69 mmol, 1.5 eq.) in dry tetrahydrofuran (25 mL) was added DIPEA (2.14 mL, 12.3 mmol, 5 eq.) and bromotri(pyrrolidin-1-yl)phosphonium (I2-fluoraneyl)pentafluorophosphate(V) (1.73 g, 3.69 mmol, 1.5 eq.). The reaction mixture was stirred at 23° C. until LC/MS indicated full consumption of the s.m. The reaction mixture was diluted with ethyl acetate and washed with aq. sat. ammoniumchloride solution, aq. sat. sodium bicarbonate solution and brine. The organic phase was dried over sodium sulfate, filtered and concentrated under reduced pressure. The crude material was purified by flash column chromatography (CH₂Cl₂/MeOH/NH₃ 90/9/1) to yield the desired tert-butyl 3-(4-(((3-fluoropyridin-2-yl)methyl)amino)oxazolo[4,5-c]pyridin-2-yl)azetidine-1-carboxylate (978 mg, 2.45 mmol, 99%).

LCMS (ESI) m/z=400.3. 1H NMR (400 MHz, DMSO) δ 8.34 (dt, J=4.7, 1.5 Hz, 1H), 7.89 (d, J=5.8 Hz, 1H), 7.67 (ddd, J=10.3, 8.3, 1.3 Hz, 1H), 7.41-7.32 (m, 2H), 6.95 (d, J=5.8 Hz, 1H), 4.85 (dd, J=5.7, 1.8 Hz, 2H), 4.31-4.24 (m, 2H), 4.18-4.06 (m, 3H), 1.39 (s, 9H) ppm.

### J. 2-(azetidin-3-yl)-N-((3-fluoropyridin-2-yl)methyl)oxazolo[4,5-c]pyridin-4-amine

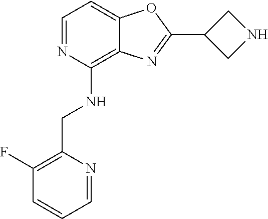 [SMILES: Fc1cccnc1CNc1nccc2oc(C3CNC3)nc12]

Tert-butyl 3-(4-(((3-fluoropyridin-2-yl)methyl)amino)oxazolo[4,5-c]pyridin-2-yl)azetidine-1-carboxylate (978 mg, 2.45 mmol, 1 eq.) was dissolved in dichloromethane (20 mL) and then treated with HCl in dioxane (4N, 6.12 mL, 24.5 mmol, 10 eq.). After full conversion of the s.m. the reaction mixture was concentrated under reduced pressure. The crude material was purified by SCX-column, eluting with 7N Ammonia in methanol to obtain the desired 2-(azetidin-3-yl)-N-((3-fluoropyridin-2-yl)methyl)oxazolo[4,5-c]pyridin-4-amine (530 mg, 1.77 mmol, 72%) as an oil.

LCMS (ESI) m/z=300.2. 1H NMR (400 MHz, DMSO) δ 8.36 (d, J=4.7 Hz, 1H), 7.93 (d, J=5.8 Hz, 1H), 7.70 (ddd, J=10.1, 8.3, 1.3 Hz, 2H), 7.39 (dt, J=8.5, 4.4 Hz, 3H), 6.99 (d, J=5.8 Hz, 2H), 4.90-4.86 (m, 3H), 4.48-4.39 (m, 1H), 4.34 (d, J=8.1 Hz, 4H) ppm.

### K. Oxazolo[4,5-c]pyridine

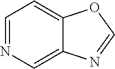

3-Aminopyridin-4-ol (1.50 g, 1 Eq, 13.6 mmol) was charged in a microwave vial and suspended in trimethyl orthoformate (14.9 mL, 136 mmol, 10 eq.). Acetic acid (1.17 mL, 20.4 mmol, 1.5 eq) was added, the vial was sealed and submitted to microwave irradiation (160° C., 30 min). The reaction mixture was concentrated and the residue was purified by flash chromatography (50% EtOAc in hexanes to 100% EtOAc) to provide the desired oxazolo[4,5-c]pyridine (1.17 g, 9.74 mmol, 72%) as a colorless oil which crystallized on standing.

LCMS (ESI) m/z=121.1. 1H NMR (400 MHz, DMSO) δ 9.12 (d, J=1.0 Hz, 1H), 8.89 (s, 1H), 8.60 (d, J=5.6 Hz, 1H), 7.89 (dd, J=5.6, 1.0 Hz, 1H) ppm.

### L. Oxazolo[4,5-c]pyridine 5-oxide

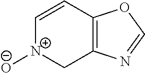

Oxazolo[4,5-c]pyridine (4.66 g, 38.8 mmol, 1 eq.) was dissolved in dichloromethane (40.0 mL). The solution was cooled to 0° C., before mCPBA (75% wt, 17.9 g, 77.6 mmol, 2.0 eq.) was added. After 2 h the ice-bath was removed and stirring was continued at 23° C. until TLC indicated full conversion of s.m. The reaction mixture was concentrated to approx. 20 mL, filtered and the filtrate was directly submitted to flash column chromatography (CH₂Cl₂/MeOH, MeOH 0-20%) affording the desired oxazolo[4,5-c]pyridine 5-oxide (4.02 g, 38.8 mmol, 76%) as a beige solid.

LCMS (ESI) m/z=137.2. 1H NMR (400 MHz, DMSO) δ 9.79 (s, 1H), 8.79 (d, J=2.9 Hz, 1H), 8.29 (d, J=1.6 Hz, 1H), 7.86 (dd, J=7.7, 2.9 Hz, 1H), 6.17 (d, J=7.7 Hz, 1H) ppm.

### M. N-((3-fluoropyridin-2-yl)methyl)oxazolo[4,5-c]pyridin-4-amine

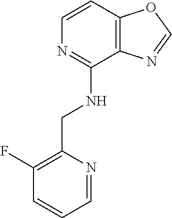 [SMILES: Fc1cccnc1CNc1nccc2ocnc12]

To a solution of oxazolo[4,5-c]pyridine 5-oxide (240 mg, 1.76 mmol, 1 eq.) and (3-fluoropyridin-2-yl)methanamine dihydrochloride (526 mg, 2.64 mmol, 1.5 eq.) in dry tetrahydrofuran (9 mL) was added DIPEA (1.14 g, 1.54 mL, 8.82 mmol, 5 eq.) and bromotri(pyrrolidin-1-yl)phosphonium hexafluorophosphate(V) (1.24 g, 2.64 mmol, 1.5 eq.). The reaction mixture was stirred at 23° C. until TLC indicated full consumption of the s.m. The reaction mixture was diluted with ethyl acetate and washed with aq. sat. ammoniumchloride solution, aq. sat. sodium bicarbonate solution and brine. The organic phase was dried over sodium sulfate, filtered and concentrated under reduced pressure. The crude material was purified by flash column chromatography (heptane/EtOAc, 0-100% EtOAc) to yield the desired N-((3-fluoropyridin-2-yl)methyl)oxazolo[4,5-c]pyridin-4-amine (315 mg, 1.29 mmol, 73%) as an off-white solid.

LCMS(ESI) m/z=245.2. 1H NMR (400 MHz, DMSO) δ 8.66 (s, 1H), 8.36 (dt, J=4.6, 1.5 Hz, 1H), 7.94 (d, J=5.8 Hz, 1H), 7.69 (ddd, J=10.1, 8.3, 1.3 Hz, 1H), 7.38 (p, J=4.4 Hz, 2H), 7.00 (d, J=5.8 Hz, 1H), 4.88 (dd, J=5.7, 1.8 Hz, 2H) ppm.

### N. 2-bromo-N-((3-fluoropyridin-2-yl)methyl)oxazolo[4,5-c]pyridin-4-amine

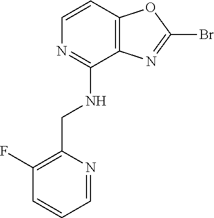 [SMILES: Fc1cccnc1CNc1nccc2oc(Br)nc12]

Under an inert atmosphere N-((3-fluoropyridin-2-yl)methyl)oxazolo[4,5-c]pyridin-4-amine (250 mg, 1 Eq, 1.02 mmol) was dissolved in tetrahydrofuran (8 mL) and cooled to −78° C. LiHMDS (360 mg, 2.15 mL, 1 molar, 2.1 Eq, 2.15 mmol) was added dropwise. The reaction mixture was allowed to warm-up to −40° C. within 60 min. Afterwards, the orange solution was re-cooled to −78° C. and then treated with N-bromosuccinimid (237 mg, 1.33 mmol, 1.3 eq.) in one portion. The reaction mixture was allowed to warm-up to 23° C. overnight. The reaction was quenches was quenched by addition of aq. sat. ammonium chloride solution and then extracted with EtOAc (3×). The combined organic phase was washed with brine, dried over sodium sulfate, filtered and concentrated under reduced pressure. The crude material was purified by flash column chromatography (heptane/EtOAc, 0-100% EtOAc) to afford the desired 2-bromo-N-((3-fluoropyridin-2-yl)methyl)oxazolo[4,5-c]pyridin-4-amine (80 mg, 0.25 mmol, 24%) as a slightly red solid.

LCMS (ESI) m/z=325.2, 323.2. 1H NMR (400 MHz, DMSO) δ 8.33 (dt, J=4.7, 1.6 Hz, 1H), 7.90 (d, J=5.8 Hz, 1H), 7.67 (ddd, J=9.9, 8.3, 1.3 Hz, 1H), 7.53 (t, J=5.7 Hz, 1H), 7.36 (dt, J=8.6, 4.4 Hz, 1H), 6.96 (d, J=5.8 Hz, 1H), 4.82 (dd, J=5.7, 1.8 Hz, 2H) ppm.

### O. N-((3-fluoropyridin-2-yl)methyl)-2-vinyloxazolo[4,5-c]pyridin-4-amine

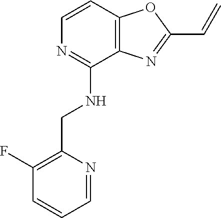 [SMILES: C=Cc1nc2c(NCc3ncccc3F)nccc2o1]

2-bromo-N-((3-fluoropyridin-2-yl)methyl)oxazolo[4,5-c]pyridin-4-amine (75 mg, 0.23 mmol, 1 eq.), Tributyl(vinyl)tin (88 μL, 0.30 mmol, 1.3 eq.) and bis(triphenylphosphine)palladium(II) chloride (16 mg, 23 μmol, 10 mol %) were suspended in 1,4-dioxane (3 mL). The reaction mixture was degassed with nitrogen for 5 min, before the reaction mixture was heated to 110° C. After TLC (approx. 1 h) showed complete conversion of the s.m., the reaction mixture was cooled to r.t. and filtered through a short pad of Celite. The filtercake was washed with dioxane. The combined filtrates were concentrated under reduced pressure. The crude material was purified by flash column chromatography (CH₂Cl₂/THF) to afford the desired N-((3-fluoropyridin-2-yl)methyl)-2-vinyloxazolo[4,5-c]pyridin-4-amine (48 mg, 0.18 mmol, 77%) as an off-white solid.

LCMS (ESI)=271.2. 1H NMR (400 MHz, DMSO) δ 8.35 (dt, J=4.6, 1.5 Hz, 1H), 7.92 (d, J=5.7 Hz, 1H), 7.68 (ddd, J=10.1, 8.4, 1.3 Hz, 1H), 7.40-7.33 (m, 2H), 6.94 (d, J=5.7 Hz, 1H), 6.81 (dd, J=17.5, 11.2 Hz, 1H), 6.36 (dd, J=17.6, 0.9 Hz, 1H), 5.93 (dd, J=11.2, 1.0 Hz, 1H), 4.85 (dd, J=5.7, 1.8 Hz, 2H) ppm.

### P. 2-bromo-1-(2-methoxyethyl)-1H-benzo[d]imidazole

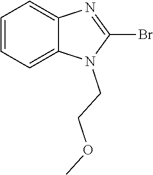

Under an inert atmosphere 2-bromo-1H-benzo[d]imidazole (3.00 g, 1 Eq, 15.2 mmol) was dissolved in N,N-Dimethylformamide (30 mL) at 23° C. to give a clear solution. Sodium hydride (0.73 g, 60% Wt, 1.2 Eq, 18.3 mmol) was added portion wise. The reaction mixture was stirred for 10 min, before 1-bromo-2-methoxyethane (2.54 g, 1.72 mL, 1.2 Eq, 18.3 mmol) was added. The reaction mixture was stirred at 23° C. for 16 hours. The reaction mixture was diluted with ethyl acetate and washed several times with water and brine. The organic phase was dried over sodium sulfate, filtered and concentrated under reduced pressure. The crude material was purified by flash column chromatography (CH₂Cl₂/MeOH) to obtain the desired 2-bromo-1-(2-methoxyethyl)-1H-benzo[d]imidazole (3.06 g, 12.0 mmol, 79%).

LCMS (ESI) m/z=257.1, 255.1. 1H NMR (400 MHz, DMSO) δ 7.66-7.55 (m, 2H), 7.25 (dtd, J=21.7, 7.4, 1.3 Hz, 2H), 4.43 (t, J=5.3 Hz, 2H), 3.68 (t, J=5.3 Hz, 2H), 3.21 (s, 3H) ppm.

### Q. 1-(2-methoxyethyl)-2-vinyl-1H-benzo[d]imidazole

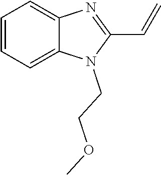

2-Bromo-1-(2-methoxyethyl)-1H-benzo[d]imidazole (1.00 g, 3.92 mmol, 1 eq.), tributyl(vinyl)tin (1.26 mL, 4.31 mmol, 1.1 eq.) and bis(triphenylphosphine)palladium(II) chloride (138 mg, 196 μmol, 5 mol %) were suspended in 1,4-dioxane (40 mL). The reaction mixture was degassed with nitrogen for 5 min, before the reaction mixture was heated to 110° C. After 1 hour TLC showed complete conversion of the s.m. The reaction mixture was cooled to r.t. and filtered through a short pad of Celite. The filtercake was washed with dioxane. The combined filtrates were concentrated under reduced pressure. The crude material was purified by flash column chromatography (CH₂Cl₂/THF) to afford the desired 1-(2-methoxyethyl)-2-vinyl-1H-benzo[d]imidazole (581 mg, 2.87 mmol, 73%) as an orange gel.

LCMS (ESI) m/z=203.3. 1H NMR (400 MHz, DMSO) δ 7.63-7.49 (m, 2H), 7.25-7.14 (m, 2H), 7.02 (dd, J=17.0, 11.0 Hz, 1H), 6.40 (dd, J=17.1, 2.2 Hz, 1H), 5.64 (dd, J=11.0, 2.2 Hz, 1H), 4.47 (t, J=5.2 Hz, 2H), 3.60 (t, J=5.2 Hz, 2H), 3.16 (s, 3H) ppm.

### R. 2-bromo-1-(cyclopropylmethyl)-1H-benzo[d]imidazole

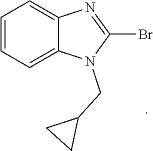 [SMILES: Brc1nc2ccccc2n1CC1CC1]

Under an inert atmosphere 2-bromo-1H-benzo[d]imidazole (3.00 g, 15.2 mmol, 1 eq.) was dissolved in N,N-dimethylformamide (30 mL) at 23° C. to give a clear solution. Sodium hydride (60% Wt, 0.73 g, 18.3 mmol, 1.2 eq.) was added portion wise. The reaction mixture was stirred for 10 min, before (bromomethyl)-cyclopropane (2.47 g, 18.3 mmol, 1.2 eq.) was added. The reaction mixture was stirred at 23° C. for 16 hours. The reaction mixture was quenched with water (5 mL) and then concentrated under reduced pressure. The concentrate was re-suspended in ethyl acetate, filtered, and again concentrated. The crude material was purified by flash column chromatography (CH₂Cl₂/MeOH) to obtain the desired 2-bromo-1-(cyclopropylmethyl)-1H-benzo[d]imidazole (2.42 g, 9.64 mmol, 63%).

LCMS (ESI) m/z=253.1, 251.1. 1H NMR (400 MHz, DMSO) δ 7.72-7.63 (m, 1H), 7.62-7.57 (m, 1H), 7.32-7.17 (m, 2H), 4.15 (d, J=7.0 Hz, 2H), 1.33-1.18 (m, 1H), 0.55-0.40 (m, 4H) ppm.

### S. 1-(cyclopropylmethyl)-2-vinyl-1H-benzo[d]imidazole

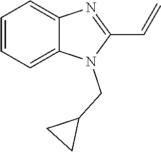

2-bromo-1-(cyclopropylmethyl)-1H-benzo[d]imidazole (1.00 g, 3.98 mmol, 1 eq.), Tributyl(vinyl)tin (1.29 mL, 4.18 mmol) and bis(triphenylphosphine)palladium(II) chloride (140 mg, 199 μmol, 5 mol %) were suspended in 1,4-dioxane (40 mL). The reaction mixture was degassed with nitrogen for 5 min, before the reaction mixture was heated to 110° C. After 1 hour TLC showed complete conversion of the s.m. The reaction mixture was cooled to 23° C. and filtered through a short pad of Celite. The filtercake was washed with dioxane. The combined filtrates were concentrated under reduced pressure. The crude material was purified by flash column chromatography (CH₂Cl₂/THF) to afford the desired 1-(cyclopropylmethyl)-2-vinyl-1H-benzo[d]imidazole (522 mg, 2.63 mmol, 66%) as a slightly yellow gel.

LCMS (ESI) m/z=199.2. 1H NMR (400 MHz, DMSO) δ 7.59 (dd, J=6.5, 2.3 Hz, 2H), 7.25-7.14 (m, 2H), 7.05 (dd, J=17.0, 10.9 Hz, 1H), 6.42 (dd, J=17.0, 2.2 Hz, 1H), 5.67 (dd, J=10.9, 2.2 Hz, 1H), 4.23 (d, J=6.9 Hz, 2H), 1.32-1.10 (m, 1H), 0.51-0.33 (m, 4H) ppm.

### T. 2-bromo-1-(2-(2-methoxyethoxy)ethyl)-1H-benzo[d]imidazole

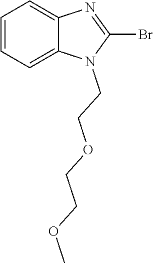 [SMILES: COCCOCCn1c(Br)nc2ccccc21]

Under an inert atmosphere 2-bromo-1H-benzo[d]imidazole (3.00 g, 15.2 mmol, 1 eq.) was dissolved in N,N-dimethylformamide (30 mL) at 23° C. to give a clear solution. Sodium hydride (60% Wt, 0.73 g, 18.3 mmol, 1.2 eq.) was added portion wise. The reaction mixture was stirred for 10 min, before 1-bromo-2-(2-methoxyethoxy)ethane (3.34 g, 18.3 mmol, 1.2 eq.) was added. The reaction mixture was stirred overnight. The reaction mixture was quenched with water (5 mL) and then concentrated under reduced pressure. The concentrate was re-suspended in ethyl acetate, filtered, and again concentrated. The crude material was purified by flash column chromatography (CH₂Cl₂/MeOH) to obtain the desired 2-bromo-1-(2-(2-methoxyethoxy)ethyl)-1H-benzo[d]imidazole (1.95 g, 6.52 mmol, 43%) as a colorless oil.

LCMS (ESI) m/z=299.2. 1H NMR (400 MHz, DMSO) δ 7.60 (ddt, J=13.3, 7.8, 0.8 Hz, 2H), 7.30-7.16 (m, 2H), 4.41 (t, J=5.4 Hz, 2H), 3.75 (t, J=5.4 Hz, 2H), 3.51-3.43 (m, 2H), 3.35-3.28 (m, 2H), 3.12 (s, 3H) ppm.

### U. 1-(2-(2-methoxyethoxy)ethyl)-2-vinyl-1H-benzo[d]imidazole

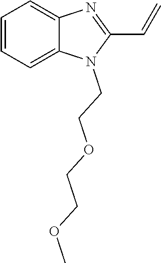 [SMILES: C=Cc1nc2ccccc2n1CCOCCOC]

2-bromo-1-(2-(2-methoxyethoxy)ethyl)-1H-benzo[d]imidazole (1.00 g, 3.34 mmol, 1 eq.), tributyl(vinyl)tin (1.13 mL, 3.38 mmol, 1.1 eq.) and bis(triphenylphosphine)palladium(II) chloride (235 mg, 334 μmol, 10 mol %) were suspended in 1,4-dioxane (30 mL). The reaction mixture was degassed with nitrogen for 5 min, before the reaction mixture was heated to 110° C. After TLC showed complete conversion of the s.m. (approx. 1 hour), the reaction mixture was cooled to 23° C. and filtered through a short pad of Celite. The filtercake was washed with dioxane. The combined filtrates were concentrated under reduced pressure. The crude material was purified by flash column chromatography (CH₂Cl₂/THF) to afford the desired 1-(2-(2-methoxyethoxy)ethyl)-2-vinyl-1H-benzo[d]imidazole (632 mg, 2.57 mmol, 77%) as a slightly yellow gel.

### V. 2-bromo-1-(2-isopropoxyethyl)-1H-benzo[d]imidazole

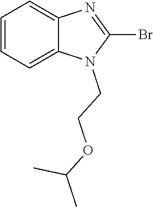 [SMILES: CC(C)OCCn1c(Br)nc2ccccc21]

Under an inert atmosphere 2-bromo-1H-benzo[d]imidazole (3.00 g, 15.2 mmol, 1 eq.) was dissolved in N,N-dimethylformamide (30 mL) at 23° C. to give a clear solution. Sodium hydride (60% Wt, 0.73 g, 18.3 mmol, 1.2 eq.) was added portion wise. The reaction mixture was stirred for 10 min, before 2-(2-bromoethoxy)propane (3.05 g, 18.3 mmol, 1.2 eq.) was added. The reaction mixture was stirred at 23° C. for 16 hours. The reaction mixture was quenched with water (5 mL) and then concentrated under reduced pressure. The concentrate was re-suspended in ethyl acetate, filtered, and again concentrated. The crude material was purified by flash column chromatography (CH₂Cl₂/MeOH) to obtain the desired 2-bromo-1-(2-isopropoxyethyl)-1H-benzo[d]imidazole (3.30 g, 11.7 mmol, 77%) as a colorless oil which crystallized on standing.

LCMS (ESI) m/z=285.3, 283.3. 1H NMR (400 MHz, DMSO) δ 7.64-7.52 (m, 2H), 7.23 (dtd, J=22.3, 7.4, 1.3 Hz, 2H), 4.37 (t, J=5.4 Hz, 2H), 3.68 (t, J=5.5 Hz, 2H), 3.43 (hept, J=6.0 Hz, 1H), 0.92 (d, J=6.0 Hz, 6H) ppm.

### W. 1-(2-isopropoxyethyl)-2-vinyl-1H-benzo[d]imidazole

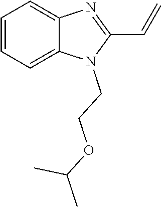 [SMILES: C=Cc1nc2ccccc2n1CCOC(C)C]

2-bromo-1-(2-isopropoxyethyl)-1H-benzo[d]imidazole (1.00 g, 3.53 mmol, 1 eq.), tributyl(vinyl)tin (1.14 mL, 3.88 mmol, 1.1 eq.) and bis(triphenylphosphine)palladium(II) chloride (248 mg, 353 μmol, 10 mol %) were suspended in 1,4-dioxane (30 mL). The reaction mixture was degassed with nitrogen for 5 min, before the reaction mixture was heated to 110° C. After TLC showed complete conversion of the s.m. (approx. 1 hour), the reaction mixture was cooled to 23° C. and filtered through a short pad of Celite. The filtercake was washed with dioxane. The combined filtrates were concentrated under reduced pressure. The crude material was purified by flash column chromatography (CH₂Cl₂/THF) to afford the desired 1-(2-isopropoxyethyl)-2-vinyl-1H-benzo[d]imidazole (660 mg, 2.87 mmol, 81%) as a slightly yellow gel.

LCMS (ESI) m/z=231.3. 1H NMR (400 MHz, DMSO) δ 7.62-7.49 (m, 2H), 7.24-7.13 (m, 2H), 7.03 (dd, J=17.1, 11.0 Hz, 1H), 6.38 (dd, J=17.1, 2.2 Hz, 1H), 4.42 (t, J=5.2 Hz, 2H), 3.62 (t, J=5.3 Hz, 2H), 3.39 (hept, J=6.0 Hz, 1H), 0.91 (d, J=6.1 Hz, 6H) ppm.

### X. 4-(2-(2-bromo-1H-benzo[d]imidazol-1-yl)ethyl)morpholine

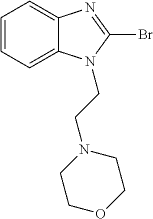 [SMILES: Brc1nc2ccccc2n1CCN1CCOCC1]

Under an inert atmosphere 2-bromo-1H-benzo[d]imidazole (1.00 g, 5.08 mmol, 1 eq.) was dissolved in dry N,N-Dimethylformamide (10 mL). The solution was cooled down to 0° C. before sodium hydride (60% Wt, 305 mg, 7.61 mmol, 1.5 eq.) was added. After 10 min 4-(2-bromoethyl)morpholine (1.48 g, 7.61 mmol, 1.5 eq.) was carefully added. The reaction mixture was slowly warmed up to 23° C. within 16 hours. The reaction mixture was quenched with water and extracted with ethyl acetate (5×). The combined organic phase was washed water (2×) and brine, dried over sodium sulfate, filtered and concentrated under reduced pressure. The crude material was purified by flash column chromatography (CH₂Cl₂/MeOH, 0-10% MeOH) affording the title 4-(2-(2-bromo-1H-benzo[d]imidazol-1-yl)ethyl)morpholine (1.25 g, 4.03 mmol, 79%).

LCMS (ESI) m/z=310.1. 1H NMR (400 MHz, DMSO) δ 7.60 (ddt, J=11.3, 7.8, 0.9 Hz, 2H), 7.24 (dtd, J=24.4, 7.3, 1.2 Hz, 2H), 4.34 (t, J=6.4 Hz, 2H), 3.51-3.48 (m, 4H), 2.63 (t, J=6.5 Hz, 2H), 2.45-2.37 (m, 4H) ppm.

### Y. 4-(2-(2-vinyl-1H-benzo[d]imidazol-1-yl)ethyl)morpholine

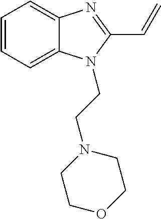 [SMILES: C=Cc1nc2ccccc2n1CCN1CCOCC1]

4-(2-(2-bromo-1H-benzo[d]imidazol-1-yl)ethyl)morpholine (555 mg, 1.79 mmol1, eq.), Tributyl(vinyl)tin (624 mg, 575 μL, 1.97 mmol, 1.1 eq.) and bis(triphenylphosphine)palladium(II) chloride (62.8 mg, 89.5 μmol, 5 mol %) were suspended in 1,4-Dioxane (15 mL). The reaction mixture was degassed with nitrogen for 5 min, before the reaction mixture was heated to 110° C. After TLC showed complete conversion of the s.m. (approx 4 hours), the reaction mixture was cooled to 23° C. and concentrated under reduced pressure. The crude material was purified by flash column chromatography (CH₂Cl₂/THF) to afford the desired 4-(2-(2-vinyl-1H-benzo[d]imidazol-1-yl)ethyl)morpholine (321 mg, 1.25 mmol, 69.7%) as a slightly yellow solid.

LCMS (ESI) m/z=258.2. 1H NMR (400 MHz, DMSO) δ 7.61-7.55 (m, 2H), 7.25-7.16 (m, 2H), 7.03 (dd, J=17.0, 11.0 Hz, 1H), 6.41 (dd, J=17.0, 2.2 Hz, 1H), 5.67 (dd, J=10.9, 2.2 Hz, 1H), 4.42 (t, J=6.4 Hz, 2H), 3.50 (t, J=4.6 Hz, 5H), 2.59 (t, J=6.4 Hz, 2H), 2.41 (dd, J=5.6, 3.7 Hz, 4H) ppm.

### Z. 1-benzyl-2-bromo-1H-benzo[d]imidazole

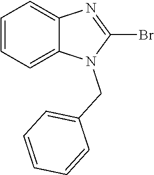 [SMILES: Brc1nc2ccccc2n1Cc1ccccc1]

Under an inert atmosphere 2-bromo-1H-benzo[d]imidazole (2.00 g, 10.2 mmol, 1 eq.) was dissolved in N,N-dimethylformamide (20 mL) at 23° C. to give a clear solution. Sodium hydride (60% Wt, 609 mg, 15.2 mmol, 1.5 eq.) was added portionwise. The reaction mixture was stirred for 10 min, before benzyl bromide (2.60 g, 15.2 mmol, 1.5 eq.) was added. The reaction mixture was stirred at 23° C. for 16 hours. The reaction mixture was quenched with water (100 mL) and extracted with ethyl acetate (3×). The combined organic phase was washed with water and brine, dried over sodium sulfate, filtered and concentrated under reduced pressure. The crude material was purified by flash column chromatography (Heptane/EtOAc) to obtain the desired 1-benzyl-2-bromo-1H-benzo[d]imidazole (2.55 g, 8.88 mmol, 88%) as an off-white solid.

LCMS (ESI) m/z=287.1.

### AA. 1-benzyl-2-vinyl-1H-benzo[d]imidazole

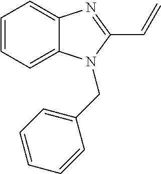 [SMILES: C=Cc1nc2ccccc2n1Cc1ccccc1]

1-benzyl-2-bromo-1H-benzo[d]imidazole (500 mg, 1.74 mmol, 1 eq.), tributyl(vinyl)tin (590 μL, 1.92 mmol, 1.1 eq.) and bis(triphenylphosphine)palladium(II) chloride (61.1 mg, 87.1 μmol, 5 mol %) were suspended in 1,4-dioxane (17 mL). The reaction mixture was degassed with nitrogen for 5 min, before the reaction mixture was heated to 110° C. After TLC showed complete conversion of the s.m. (approx. 2 hour), the reaction mixture was cooled to 23° C. and filtered through a short pad of Celite. The filtercake was washed with dioxane. The combined filtrates were concentrated under reduced pressure. The crude material was purified by flash column chromatography (CH₂Cl₂/THF) to afford the desired 1-benzyl-2-vinyl-1H-benzo[d]imidazole (230 mg, 982 μmol, 56%) as a slightly yellow gel.

LCMS (ESI) m/z=235.2. 1H NMR (400 MHz, DMSO) δ 7.66-7.60 (m, 1H), 7.58-7.53 (m, 1H), 7.34-7.28 (m, 2H), 7.27-7.23 (m, 1H), 7.23-7.17 (m, 2H), 7.14-7.10 (m, 2H), 7.08-7.01 (m, 1H), 6.43 (dd, J=17.0, 2.1 Hz, 1H), 5.66 (dd, J=10.9, 2.1 Hz, 1H), 5.61 (s, 2H) ppm.

### BB. 2-bromo-1-(3-methoxybenzyl)-1H-benzo[d]imidazole

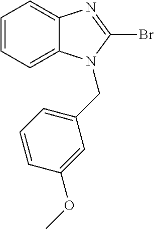 [SMILES: COc1cccc(Cn2c(Br)nc3ccccc32)c1]

Under an inert atmosphere 2-bromo-1H-benzo[d]imidazole (2.00 g, 10.2 mmol, 1 eq.) was dissolved in N,N-dimethylformamide (20 mL) at 23° C. to give a clear solution. Sodium hydride (60% Wt, 609 mg, 15.2 mmol, 1.5 eq.) was added portionwise. The reaction mixture was stirred for 10 min, before 1-(bromomethyl)-3-methoxybenzene (2.13 mL, 15.2 mmol, 1.5 eq.) was added. The reaction mixture was stirred at 23° C. for 16 hours. The reaction mixture was quenched with water (100 mL) and extracted with ethyl acetate (3×). The combined organic phase was washed with water and brine, dried over sodium sulfate, filtered and concentrated under reduced pressure. The crude material was purified by flash column chromatography (Heptane/EtOAc) to obtain the desired 2-bromo-1-(3-methoxybenzyl)-1H-benzo[d]imidazole (1.43 g, 4.51 mmol, 44%) as an off-white solid.

LCMS (ESI) m/z=317.2. 1H NMR (400 MHz, DMSO) δ 7.69-7.56 (m, 2H), 7.32-7.20 (m, 3H), 6.87 (ddd, J=8.2, 2.7, 0.9 Hz, 1H), 6.80 (t, J=2.1 Hz, 1H), 6.68 (ddd, J=7.6, 1.7, 0.9 Hz, 1H), 5.50 (s, 2H), 3.71 (s, 3H) ppm.

### CC. 1-(3-methoxybenzyl)-2-vinyl-1H-benzo[d]imidazole

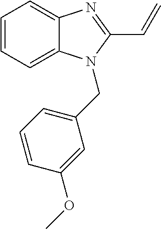 [SMILES: C=Cc1nc2ccccc2n1Cc1cccc(OC)c1]

2-bromo-1-(3-methoxybenzyl)-1H-benzo[d]imidazole (507 mg, 1.60 mmol, 1 eq.), tributyl(vinyl)tin (514 μL, 1.76 mmol, 1.1 eq.) and bis(triphenylphosphine)palladium(II) chloride (56.1 mg, 79.9 μmol, 5 mol %) were suspended in 1,4-dioxane (17 mL). The reaction mixture was degassed with nitrogen for 5 min, before the reaction mixture was heated to 110° C. After TLC showed complete conversion of the s.m. (approx. 2 hour), the reaction mixture was cooled to 23° C. and filtered through a short pad of Celite. The filtercake was washed with dioxane. The combined filtrates were concentrated under reduced pressure. The crude material was purified by flash column chromatography (CH₂Cl₂/THF) to afford the desired 1-(3-methoxybenzyl)-2-vinyl-1H-benzo[d]imidazole (280 mg, 1.06 mmol, 66%) as a slightly yellow gel.

LCMS (ESI) m/z=265.2. 1H NMR (400 MHz, DMSO) δ 7.68-7.50 (m, 2H), 7.20 (tt, J=4.7, 2.8 Hz, 3H), 7.06 (ddd, J=17.0, 11.0, 1.1 Hz, 1H), 6.82 (dd, J=8.4, 2.5 Hz, 1H), 6.71 (t, J=2.0 Hz, 1H), 6.43 (dt, J=17.0, 1.6 Hz, 1H), 5.66 (dt, J=10.9, 1.6 Hz, 1H), 5.57 (s, 2H), 3.68 (d, J=1.1 Hz, 3H) ppm.

### DD. 1-bromo-1-(3-chlorobenzyl)-1H-benzo[d]imidazole

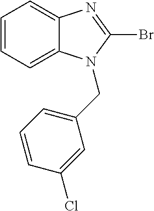 [SMILES: Clc1cccc(Cn2c(Br)nc3ccccc32)c1]

Under an inert atmosphere 2-bromo-1H-benzo[d]imidazole (2.00 g, 10.2 mmol, 1 eq.) was dissolved in N,N-dimethylformamide (20 mL) at 23° C. to give a clear solution. Sodium hydride (60% Wt, 609 mg, 15.2 mmol, 1.5 eq.) was added portionwise. The reaction mixture was stirred for 10 min, before 1-(bromomethyl)-3-chlorobenzene (2.00 mL, 15.2 mmol, 1.5 eq.) was added. The reaction mixture was stirred at 23° C. for 16 hours. The reaction mixture was quenched with water (100 mL) and extracted with ethyl acetate (3×). The combined organic phase was washed with water and brine, dried over sodium sulfate, filtered and concentrated under reduced pressure. The crude material was purified by flash column chromatography (Heptane/EtOAc) to obtain the desired 2-bromo-1-(3-chlorobenzyl)-1H-benzo[d]imidazole (2.57 g, 7.99 mmol, 79%) as an off-white solid.

LCMS (ESI) m/z=321.1. 1H NMR (400 MHz, DMSO) δ 7.69-7.57 (m, 2H), 7.39-7.33 (m, 2H), 7.31-7.19 (m, 3H), 7.10-7.01 (m, 1H), 5.54 (s, 2H) ppm.

### EE. 1-(3-chlorobenzyl)-2-vinyl-1H-benzo[d]imidazole

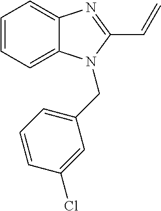 [SMILES: C=Cc1nc2ccccc2n1Cc1cccc(Cl)c1]

2-bromo-1-(3-chlorobenzyl)-1H-benzo[d]imidazole (800 mg, 2.49 mmol, 1 eq.), tributyl(vinyl)tin (800 μL, 2.74 mmol, 1.1 eq.) and bis(triphenylphosphine)palladium(II) chloride (87.3 mg, 124 μmol, 5 mol %) were suspended in 1,4-dioxane (18 mL). The reaction mixture was degassed with nitrogen for 5 min, before the reaction mixture was heated to 110° C. After TLC showed complete conversion of the s.m. (approx. 2 hour), the reaction mixture was cooled to 23° C. and filtered through a short pad of Celite. The filtercake was washed with dioxane. The combined filtrates were concentrated under reduced pressure. The crude material was purified by flash column chromatography (CH₂Cl₂/THF) to afford the desired 1-(3-chlorobenzyl)-2-vinyl-1H-benzo[d]imidazole (451 mg, 1.68 mmol, 68%) as a slightly yellow gel.

1H NMR (400 MHz, DMSO) δ 7.67-7.61 (m, 1H), 7.59-7.55 (m, 1H), 7.35-7.30 (m, 2H), 7.24-7.18 (m, 3H), 7.11-6.96 (m, 2H), 6.44 (dd, J=17.0, 2.1 Hz, 1H), 5.68 (dd, J=11.0, 2.1 Hz, 1H), 5.64 (s, 2H) ppm.

### FF. 3-(benzyloxy)-N-(4-hydroxypyridin-3-yl)propanamide

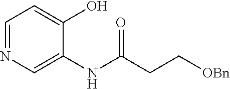 [SMILES: O=C(CCOCc1ccccc1)Nc1cnccc1O]

To a solution of 3-aminopyridin-4-ol (3.06 g, 27.7 mmol, 1 eq.) in DMF (80 mL) was added 3-(benzyloxy)propanoic acid (5.00 g, 27.7 mmol, 1 eq.), triethylamine (5.62 g, 7.73 mL, 55.5 mmol, 2 eq.) and HATU (12.7 g, 33.3 mmol, 1.2 eq.). The reaction mixture was stirred at 23° C. for 16 hours. The solution was diluted with water and extracted with ethyl acetate (3×). The combined organic phase was washed with water and brine, dried over sodium sulfate, filtered and concentrated under reduced pressure. The crude oil was purified by flash column chromatography (CH₂Cl₂/MeOH, 0-15% MeOH) to give the desired 3-(benzyloxy)-N-(4-hydroxypyridin-3-yl)propanamide (6.78 g, 24.9 mmol, 90%) as a white foam.

LCMS (ESI) m/z=273.1. 1H NMR (400 MHz, DMSO) δ 9.33 (s, 1H), 8.82 (d, J=1.5 Hz, 1H), 7.77 (dd, J=7.0, 1.6 Hz, 1H), 7.39-7.31 (m, 4H), 7.29-7.21 (m, 1H), 6.43 (d, J=7.0 Hz, 1H), 4.50 (s, 2H), 3.71 (t, J=6.1 Hz, 2H), 2.74 (t, J=6.1 Hz, 2H) ppm.

### GG. 2-(2-(benzyloxy)ethyl)oxazolo[4,5-c]pyridine

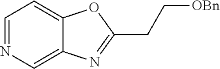 [SMILES: c1ccc(COCCc2nc3cnccc3o2)cc1]

To polymer supported triphenyl phosphine (polymer supported, loading 1.6 mmol/g, 12.6 g) in dichloromethane (100 mL) was added hexachloroethane (2.97 g, 12.5 mmol, 1.25 eq.) and triethylamine (5.07 g, 6.99 mL, 50.1 mmol, 5 eq.). The suspension was stirred for 5 min, before solid 3-(benzyloxy)-N-(4-hydroxypyridin-3-yl)propanamide (2.73 g, 10.0 mmol, 1 eq.) was added. The mixture was stirred 1 h at r.t. Another portion of hexachloroethane (1.78 g, 0.75 eq.) and triethylamine (2.33 mL) was added and stirring was continued at 23° C. for 16 hours. The reaction mixture was diluted with dichloromethane and acetonitrile and then filtered. The filtrate was concentrated under reduced pressure and then purified by flash column chromatography (Heptane/EtOAc, 0-100% EtOAc) to obtain the desired 2-(2-(benzyloxy)ethyl)oxazolo[4,5-c]pyridine (1.68 g, 6.61 mmol, 66%) as a yellow oil which crystallized on standing.

LCMS (ESI) m/z=255.2. 1H NMR (400 MHz, DMSO) δ 9.01 (d, J=0.9 Hz, 1H), 8.54 (d, J=5.5 Hz, 1H), 7.80 (dd, J=5.5, 1.0 Hz, 1H), 7.35-7.22 (m, 5H), 4.53 (s, 2H), 3.93 (t, J=6.3 Hz, 2H), 3.30 (t, J=6.4 Hz, 2H) ppm.

### HH. 2-(2-(benzyloxy)ethyl)oxazolo[4,5-c]pyridine 5-oxide

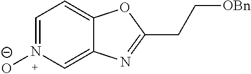 [SMILES: [O-][n+]1ccc2oc(CCOCc3ccccc3)nc2c1]

2-(2-(benzyloxy)ethyl)oxazolo[4,5-c]pyridine (1.68 g, 6.61 mmol, 1 eq.) was dissolved in dichloromethane (60 mL). The solution was cooled to 0° C., before mCPBA (75% Wt, 3.04 g, 13.2 mmol, 2 eq.) was added. After 2 h the ice-bath was removed and stirring was continued at 23° C. until LCMS indicated full conversion of s.m. The reaction mixture was diluted with dichloromethane and washed with aq. sat. sodium bicarbonate solution and brine, dried over sodium sulfate, filtered and concentrated under reduced pressure. The crude material was re-crystallized in methanol. The solid was filtered-off, washed with methanol and dried under vacuum to yield the desired 2-(2-(benzyloxy)ethyl)oxazolo[4,5-c]pyridine 5-oxide (1.30 g, 4.81 mmol, 73%) as an off-white solid.

LCMS (ESI) m/z=271.3. 1H NMR (400 MHz, DMSO) δ 8.79 (d, J=1.8 Hz, 1H), 8.21 (dd, J=7.0, 1.8 Hz, 1H), 7.81 (d, J=7.0 Hz, 1H), 7.35-7.20 (m, 5H), 4.51 (s, 2H), 3.89 (t, J=6.3 Hz, 2H), 3.27 (t, J=6.3 Hz, 2H) ppm.

### II. 2-(2-(benzyloxy)ethyl)-N-((3-fluoropyridin-2-yl)methyl)oxazolo[4,5-c]pyridin-4-amine

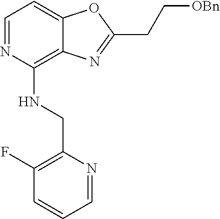 [SMILES: Fc1cccnc1CNc1nccc2oc(CCOCc3ccccc3)nc12]

To a solution of 2-(2-(benzyloxy)ethyl)oxazolo[4,5-c]pyridine 5-oxide (1.30 g, 4.81 mmol, 1 eq.) and (3-fluoropyridin-2-yl)methanamine dihydrochloride (1.44 g, 7.21 mmol, 1.5 eq.) in dry tetrahydrofuran (50 mL) was added N,N-diisopropylethylamine (3.11 g, 4.19 mL, 24.0 mmol, 5 eq.) and bromotri(pyrrolidin-1-yl)phosphonium (I2-fluoraneyl)pentafluoro-phosphate(V) (3.37 g, 7.21 mmol, 1.5 eq.). The reaction mixture was stirred at 23° C. until LC/MS indicated full consumption of the s.m. The reaction mixture was diluted with ethyl acetate and washed with aq. sat. ammoniumchloride solution, aq. sat. sodium bicarbonate solution and brine. The organic phase was dried over sodium sulfate, filtered and concentrated under reduced pressure. The crude material was purified by flash column chromatography (CH₂Cl₂/MeOH/NH₃ 90/9/1) to yield the desired 2-(2-(benzyloxy)ethyl)-N-((3-fluoropyridin-2-yl)methyl)oxazolo[4,5-c]pyridin-4-amine (1.76 g, 4.65 mmol, 97%).

LCMS (ESI) m/z=379.4. 1H NMR (400 MHz, DMSO) δ 8.34 (dt, J=4.7, 1.5 Hz, 1H), 7.87 (d, J=5.8 Hz, 1H), 7.67 (ddd, J=10.1, 8.4, 1.3 Hz, 1H), 7.36 (dt, J=8.6, 4.4 Hz, 1H), 7.33-7.23 (m, 5H), 7.19 (t, J=5.6 Hz, 1H), 6.91 (d, J=5.7 Hz, 1H), 4.83 (dd, J=5.6, 1.7 Hz, 2H), 4.51 (s, 2H), 3.88 (t, J=6.4 Hz, 2H), 3.22 (t, J=6.4 Hz, 2H) ppm.

### JJ. 2-(4-(((3-fluoropyridin-2-yl)methyl)amino)oxazolo[4,5-c]pyridin-2-yl)ethan-1-ol

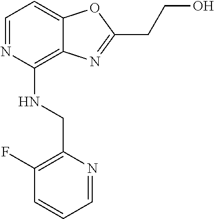 [SMILES: OCCc1nc2c(NCc3ncccc3F)nccc2o1]

2-(2-(benzyloxy)ethyl)-N-((3-fluoropyridin-2-yl)methyl)oxazolo[4,5-c]pyridin-4-amine (500 mg, 1.32 mmol, 1 eq.) was dissolved in dichloromethane (12 mL). The colorless solution was treated with Boron tribromide dimethyl sulfide complex (1M in dichloromethane, 3.96 mL, 3.96 mmol, 1 eq.) in two portion (2 eq. immediately+1 eq. after 30 min). The reaction mixture was stirred at 23° C. until TLC indicated full conversion of s.m. The reaction mixture was quenched by slow addition of aq. sat. sodium bicarbonate solution. The mixture was stirred for 30 min, followed by separation of the two phases. The aq. phase was extracted with dichloromethane (3×). The combined organic phase was dried over sodium sulfate, filtered and concentrated under reduced pressure. The crude material was purified by flash column chromatography (CH₂Cl₂/MeOH, MeOH 0-20%) obtaining the desired 2-(4-(((3-fluoropyridin-2-yl)methyl)amino)oxazolo[4,5-c]pyridin-2-yl)ethan-1-ol (180 mg, 624 μmol, 47%) as a colorless oil which crystallized on standing.

LCMS (ESI) m/z=289.2. 1H NMR (400 MHz, DMSO) δ 8.37 (dt, J=4.7, 1.5 Hz, 1H), 7.87 (d, J=5.8 Hz, 1H), 7.69 (ddd, J=10.3, 8.3, 1.3 Hz, 1H), 7.38 (dt, J=8.6, 4.4 Hz, 1H), 7.20 (t, J=5.6 Hz, 1H), 6.93 (d, J=5.7 Hz, 1H), 4.93 (t, J=5.5 Hz, 1H), 4.85 (dd, J=5.7, 1.8 Hz, 2H), 3.87 (q, J=6.1 Hz, 2H), 3.07 (t, J=6.4 Hz, 2H) ppm.

### KK. N-((3-fluoropyridin-2-yl)methyl)-2-(2-iodoethyl)oxazolo[4,5-c]pyridin-4-amine

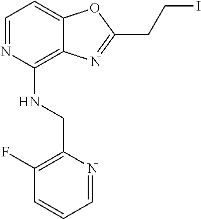 [SMILES: Fc1cccnc1CNc1nccc2oc(CCI)nc12]

Triphenylphosphine (1.11 g, 4.24 mmol, 1.3 eq.) was dissolved in dichloromethane (7 mL). Iodine (1.08 g, 4.24 mmol, 1.3 eq.) was added in small portions. 10 min later imidazole (300 mg, 4.40 mmol, 1.35 eq.) was added. After additional 10 min the reaction mixture was cooled down to 0° C. and 2-(4-(((3-fluoropyridin-2-yl)methyl)amino)oxazolo[4,5-c]pyridin-2-yl)ethan-1-ol (940 mg, 1 Eq, 3.26 mmol) was added. After TLC showed complete conversion of the s.m. aq. sat. sodium thiosulfate solution was added to quench the reaction. The reaction mixture was separated and the aqueous phase was re-extracted with dichloromethane (3×). The combined organic phase was washed with water and brine, dried over sodium sulfate, filtered and concentrated under reduced pressure.

The crude material was purified by flash column chromatography (Heptane/EtOAc) to obtain the desired N-((3-fluoropyridin-2-yl)methyl)-2-(2-iodoethyl)oxazolo[4,5-c]pyridin-4-amine (450 mg, 1.13 mmol, 35%) as a white solid.

1H NMR (400 MHz, DMSO) δ 8.35 (dt, J=4.7, 1.5 Hz, 1H), 7.89 (d, J=5.8 Hz, 1H), 7.68 (ddd, J=10.1, 8.3, 1.3 Hz, 1H), 7.40-7.31 (m, 2H), 6.96 (d, J=5.8 Hz, 1H), 4.85 (dd, J=5.6, 1.8 Hz, 2H), 3.73-3.46 (m, 4H) ppm.

### EXAMPLE COMPOUNDS

### Example Compound No. 1: N-((3-fluoropyridin-2-yl)methyl)-2-(2-((2-(1-(2-methoxyethyl)-1H-benzo[d]imidazol-2-yl)ethyl)amino)ethyl)oxazolo[4,5-c]pyridin-4-amine

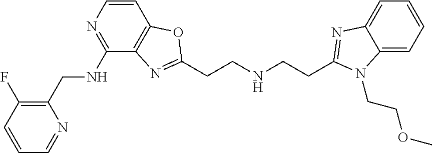 [SMILES: COCCn1c(CCNCCc2nc3c(NCc4ncccc4F)nccc3o2)nc2ccccc21]

2-(2-aminoethyl)-N-((3-fluoropyridin-2-yl)methyl)oxazolo[4,5-c]pyridin-4-amine (303 mg, 1.05 mmol, 1 eq.), 1-(2-methoxyethyl)-2-vinyl-1H-benzo[d]imidazole (213 mg, 1.05 mmol, 1 eq.) and ammonium acetate (97.6 mg, 1.27 mmol, 1.2 eq.) were suspended in acetonitrile (10 mL). The reaction mixture was heated to 50° C. for 24 hours. The reaction mixture was dry-loaded on silica and purified by flash column chromatography (CH₂Cl₂/MeOH 0-20% MeOH) obtaining N-((3-fluoropyridin-2-yl)methyl)-2-(2-((2-(1-(2-methoxyethyl)-1H-benzo[d]imidazol-2-yl)ethyl)amino)ethyl)oxazolo[4,5-c]pyridin-4-amine (205 mg, 419 μmol, 39.7%) as a brownish oil.

LCMS (ESI) m/z=490.8. ¹H NMR (400 MHz, DMSO) δ 8.36 (dt, J=4.7, 1.5 Hz, 1H), 7.88 (d, J=5.8 Hz, 1H), 7.69 (ddd, J=10.3, 8.3, 1.3 Hz, 1H), 7.52-7.45 (m, 2H), 7.42-7.34 (m, 1H), 7.21-7.07 (m, 3H), 6.91 (d, J=5.8 Hz, 1H), 4.85 (dd, J=5.6, 1.7 Hz, 2H), 4.35 (t, J=5.3 Hz, 2H), 3.61 (t, J=5.2 Hz, 2H), 3.17 (s, 3H), 3.10 (d, J=11.8 Hz, 6H), 3.02 (td, J=6.1, 1.7 Hz, 2H) ppm.

### Example Compound No. 2: 2-(2-((2-(1-(cyclopropylmethyl)-1H-benzo[d]imidazol-2-yl)ethyl)amino)ethyl)-N-((3-fluoropyridin-2-yl)methyl)oxazolo[4,5-c]pyridin-4-amine

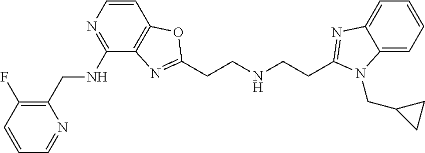 [SMILES: Fc1cccnc1CNc1nccc2oc(CCNCCc3nc4ccccc4n3CC3CC3)nc12]

2-(2-Aminoethyl)-N-((3-fluoropyridin-2-yl)methyl)oxazolo[4,5-c]pyridin-4-amine (200 mg, 696 μmol, 1 eq.), 1-(cyclopropylmethyl)-2-vinyl-1H-benzo[d]imidazole (138 mg, 696 μmol, 1 eq.) and ammonium acetate (64.4 mg, 835 μmol, 1.2 eq.) were suspended in acetonitrile (7 mL). The reaction mixture was heated to 50° C. for 24 hours. The reaction mixture was dry-loaded on silica and purified by flash column chromatography (CH₂Cl₂/MeOH 0-20% MeOH) obtaining 2-(2-((2-(1-(cyclopropylmethyl)-1H-benzo[d]imidazol-2-yl)ethyl)amino)ethyl)-N-((3-fluoropyridin-2-yl)methyl)oxazolo[4,5-c]pyridin-4-amine (132 mg, 272 μmol, 39%) as a brownish oil.

LCMS (ESI) m/z=486.5. ¹H NMR (400 MHz, DMSO) δ 8.34 (dt, J=4.7, 1.5 Hz, 1H), 7.86 (d, J=5.7 Hz, 1H), 7.67 (ddd, J=10.3, 8.3, 1.3 Hz, 1H), 7.52-7.44 (m, 2H), 7.36 (dt, J=8.6, 4.4 Hz, 1H), 7.18-7.07 (m, 3H), 6.90 (d, J=5.8 Hz, 1H), 4.83 (dd, J=5.7, 1.8 Hz, 2H), 4.07 (d, J=6.9 Hz, 2H), 3.09 (d, J=6.0 Hz, 6H), 3.01 (td, J=6.5, 1.6 Hz, 2H), 1.17 (tq, J=9.8, 3.6 Hz, 1H), 0.49-0.34 (m, 4H) ppm.

### Example Compound No. 3: N-((3-fluoropyridin-2-yl)methyl)-2-(2-((2-(1-(2-(2-methoxyethoxy)ethyl)-1H-benzo[d]imidazol-2-yl)ethyl)amino)ethyl)oxazolo[4,5-c]pyridin-4-amine

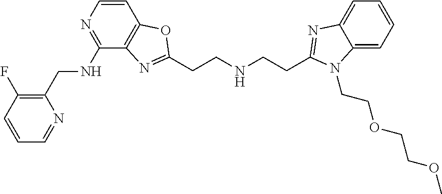 [SMILES: COCCOCCn1c(CCNCCc2nc3c(NCc4ncccc4F)nccc3o2)nc2ccccc21]

2-(2-Aminoethyl)-N-((3-fluoropyridin-2-yl)methyl)oxazolo[4,5-c]pyridin-4-amine (200 mg, 696 μmol, 1 eq.), 1-(2-(2-methoxyethoxy)ethyl)-2-vinyl-1H-benzo[d]imidazole (171 mg, 696 μmol, 1 eq.) and ammonium acetate (64.4 mg, 835 μmol, 1.2 eq.) were suspended in acetonitrile (7 mL). The reaction mixture was heated to 50° C. for 24 hours. The reaction mixture was dry-loaded on silica and purified by flash column chromatography (CH₂Cl₂/MeOH 0-20% MeOH) obtaining N-((3-fluoropyridin-2-yl)methyl)-2-(2-((2-(1-(2-(2-methoxyethoxy)ethyl)-1H-benzo[d]imidazol-2-yl)ethyl)amino)ethyl)-oxazolo[4,5-c]pyridin-4-amine (25 mg, 47 μmol, 7%) as a brownish oil.

LCMS (ESI) m/z=534.7. ¹H NMR (400 MHz, DMSO) δ 8.34 (dt, J=4.7, 1.5 Hz, 1H), 7.87 (d, J=5.8 Hz, 1H), 7.67 (ddd, J=10.1, 8.4, 1.3 Hz, 1H), 7.48 (td, J=7.7, 1.3 Hz, 2H), 7.36 (dt, J=8.6, 4.5 Hz, 1H), 7.18-7.07 (m, 3H), 6.90 (d, J=5.8 Hz, 1H), 4.83 (dd, J=5.6, 1.8 Hz, 2H), 4.34 (t, J=5.3 Hz, 2H), 3.68 (t, J=5.3 Hz, 2H), 3.45-3.36 (m, 2H), 3.33-3.23 (m, 2H), 3.16-3.12 (m, 6H), 3.10 (s, 3H), 3.06 (t, J=4.2 Hz, 3H) ppm.

### Example Compound No. 4: N-((3-fluoropyridin-2-yl)methyl)-2-(2-((2-(1-(2-isopropoxyethyl)-1H-benzo[d]imidazol-2-yl)ethyl)amino)ethyl)oxazolo[4,5-c]pyridin-4-amine

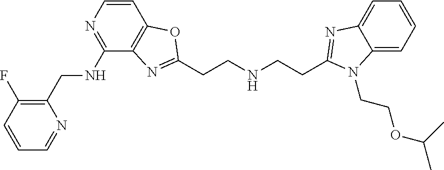 [SMILES: CC(C)OCCn1c(CCNCCc2nc3c(NCc4ncccc4F)nccc3o2)nc2ccccc21]

2-(2-Aminoethyl)-N-((3-fluoropyridin-2-yl)methyl)oxazolo[4,5-c]pyridin-4-amine (200 mg, 1 696 μmol, 1 eq.), 1-(2-isopropoxyethyl)-2-vinyl-1H-benzo[d]imidazole (160 mg, 696 μmol, 1 eq.) and ammonium acetate (64.4 mg, 835 μmol, 1.2 eq.) were suspended in acetonitrile (7 mL). The reaction mixture was heated to 50° C. for 24 hours. The reaction mixture was dry-loaded on silica and purified by flash column chromatography (CH₂Cl₂/MeOH 0-20% MeOH) obtaining N-((3-fluoropyridin-2-yl)methyl)-2-(2-((2-(1-(2-isopropoxyethyl)-1H-benzo[d]imidazol-2-yl)ethyl)amino)ethyl)oxazolo[4,5-c]pyridin-4-amine (88 mg, 0.17 mmol, 24%) as a dark yellow oil.

LCMS (ESI) m/z=518.5. ¹H NMR (400 MHz, DMSO) δ 8.34 (dt, J=4.7, 1.5 Hz, 1H), 7.86 (d, J=5.8 Hz, 1H), 7.67 (ddd, J=9.9, 8.3, 1.3 Hz, 1H), 7.49-7.44 (m, 2H), 7.36 (dt, J=8.6, 4.4 Hz, 1H), 7.17-7.06 (m, 3H), 6.89 (d, J=5.8 Hz, 1H), 4.83 (dd, J=5.6, 1.7 Hz, 2H), 4.30 (t, J=5.3 Hz, 2H), 3.61 (t, J=5.3 Hz, 2H), 3.38 (p, J=6.1 Hz, 1H), 3.12-3.00 (m, 8H), 0.90 (d, J=6.0 Hz, 6H) ppm.

### Example Compound No. 5: N-((3-fluoropyridin-2-yl)methyl)-2-(1-(2-(1-(2-methoxyethyl)-1H-benzo[d]imidazol-2-yl)ethyl)azetidin-3-yl)oxazolo[4,5-c]pyridin-4-amine

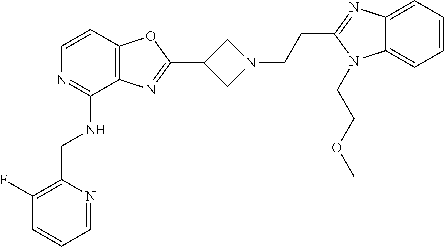 [SMILES: COCCn1c(CCN2CC(c3nc4c(NCc5ncccc5F)nccc4o3)C2)nc2ccccc21]

2-(Azetidin-3-yl)-N-((3-fluoropyridin-2-yl)methyl)oxazolo[4,5-c]pyridin-4-amine (100 mg, 334 μmol, 1 eq.), 1-(2-methoxyethyl)-2-vinyl-1H-benzo[d]imidazole (67.6 mg, 334 μmol, 1 eq.) and ammonium acetate (30.9 mg, 401 μmol, 1.2 eq.) were suspended in acetonitrile (3 mL). The reaction mixture was heated to 50° C. for 24 hours. The reaction mixture was dry-loaded on silica and purified by flash column chromatography (CH₂Cl₂/MeOH 0-20% MeOH) obtaining the desired N-((3-fluoropyridin-2-yl)methyl)-2-(1-(2-(1-(2-methoxyethyl)-1H-benzo[d]imidazol-2-yl)ethyl)azetidin-3-yl)oxazolo[4,5-c]pyridin-4-amine (60 mg, 0.12 mmol, 36%) as an colorless oil.

LCMS (ESI) m/z=502.6. ¹H NMR (400 MHz, DMSO) δ 8.34 (dt, J=4.7, 1.5 Hz, 1H), 7.87 (d, J=5.8 Hz, 1H), 7.56-7.44 (m, 2H), 7.40-7.31 (m, 1H), 7.23 (t, J=5.6 Hz, 1H), 7.19-7.09 (m, 2H), 6.92 (d, J=5.8 Hz, 1H), 4.83 (dd, J=5.6, 1.7 Hz, 2H), 4.37 (t, J=5.2 Hz, 2H), 3.93 (p, J=7.3 Hz, 1H), 3.66 (t, J=7.5 Hz, 2H), 3.62 (t, J=5.2 Hz, 2H), 3.44 (t, J=6.9 Hz, 2H), 3.17 (s, 3H), 2.99-2.85 (m, 4H) ppm.

### Example Compound No. 6: N-((3-fluoropyridin-2-yl)methyl)-2-(1-(2-(1-(2-(2-methoxyethoxy)ethyl)-1H-benzo[d]imidazol-2-yl)ethyl)azetidin-3-yl)oxazolo[4,5-c]pyridin-4-amine

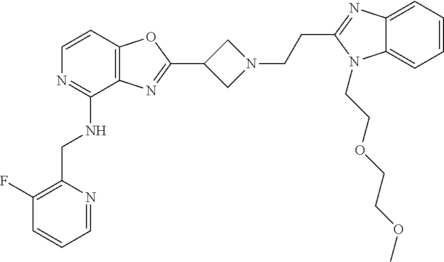 [SMILES: COCCOCCn1c(CCN2CC(c3nc4c(NCc5ncccc5F)nccc4o3)C2)nc2ccccc21]

2-(Azetidin-3-yl)-N-((3-fluoropyridin-2-yl)methyl)oxazolo[4,5-c]pyridin-4-amine (100 mg, 334 μmol, 1 eq.), 1-(2-(2-methoxyethoxy)ethyl)-2-vinyl-1H-benzo[d]imidazole (82.3 mg, 334 μmol, 1 eq.) and ammonium acetate (30.9 mg, 401 μmol, 1.2 eq) were suspended in acetonitrile (3 mL). The reaction mixture was heated to 50° C. for 24 hours. The reaction mixture was dry-loaded on silica and then purified by flash column chromatography (CH₂Cl₂/MeOH 0-20% MeOH) obtaining the desired N-((3-fluoropyridin-2-yl)methyl)-2-(1-(2-(1-(2-(2-methoxyethoxy)ethyl)-1H-benzo[d]imidazol-2-yl)ethyl) azetidin-3-yl)oxazolo[4,5-c]pyridin-4-amine (57 mg, 0.10 mmol, 31%) as a colorless oil.

LCMS (ESI) m/z=546.7. ¹H NMR (400 MHz, DMSO) δ 8.34 (dt, J=4.7, 1.5 Hz, 1H), 7.57-7.45 (m, 2H), 7.36 (dt, J=8.6, 4.4 Hz, 1H), 7.23 (t, J=5.6 Hz, 1H), 7.18-7.09 (m, 2H), 6.92 (d, J=5.7 Hz, 1H), 4.83 (dd, J=5.6, 1.8 Hz, 2H), 4.37 (t, J=5.2 Hz, 2H), 3.94 (p, J=7.3 Hz, 1H), 3.73-3.64 (m, 4H), 3.50-3.39 (m, 4H), 3.12 (s, 3H), 3.01-2.87 (m, 4H) ppm. —CH₂— signal covered by H₂O signal.

### Example Compound No. 7: N-((3-fluoropyridin-2-yl)methyl)-2-(2-((2-(1-(2-methoxyethyl)-6,7-dihydro-1H-[1,4]dioxino-[2′,3′:4,5]benzo[1,2-d]imidazol-2-yl)ethyl)amino)ethyl)oxazolo[4,5-c]pyridin-4-amine

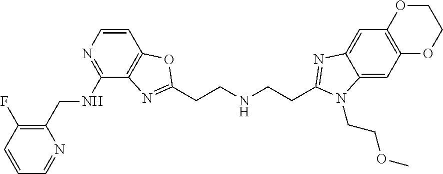 [SMILES: COCCn1c(CCNCCc2nc3c(NCc4ncccc4F)nccc3o2)nc2cc3c(cc21)OCCO3]

2-(1-(2-methoxyethyl)-6,7-dihydro-1H-[1,4]dioxino[2′,3′:4,5]benzo[1,2-d]imidazol-2-yl) ethan-1-amine dihydrochloride (51.8 mg, 148 μmol, 1 eq.) and sodium hydroxide (63 mg, 30% Wt, 474 μmol, 3.2 Eq) were dissolved in water (1.5 mL). N-((3-fluoropyridin-2-yl)methyl)-2-vinyloxazolo[4,5-c]pyridin-4-amine (40.0 mg, 148 μmol, 1 eq.) was added and the reaction mixture was heated to 80° C. for 48 hour. The reaction mixture was allowed to cool down to 23° C. before the pH was adjusted to pH=7 using 3N hydrochloric acid. The reaction mixture was concentrated and purified by flash column chromatography (CH₂Cl₂/MeOH, 0→20% MeOH) followed by SCX-columns (1-7N NH₃ in MeOH) affording the desired N-((3-fluoropyridin-2-yl)methyl)-2-(2-((2-(1-(2-methoxyethyl)-6,7-dihydro-1H-[1,4]dioxino[2′,3′:4,5]-benzo[1,2-d]imidazol-2-yl)ethyl)amino)ethyl)oxazolo[4,5-c]pyridin-4-amine (15 mg, 27 μmol, 19%) as a pale yellow solid.

LCMS (ESI) m/z=548.6. ¹H NMR (400 MHz, DMSO) δ 8.20 (dt, J=4.7, 1.5 Hz, 1H), 7.87 (d, J=5.5 Hz, 1H), 7.73 (ddd, J=9.9, 8.3, 1.3 Hz, 1H), 7.36 (dt, J=8.6, 4.4 Hz, 1H), 6.94 (d, J=12.8 Hz, 2H), 6.56 (d, J=5.5 Hz, 1H), 5.63 (d, J=1.7 Hz, 2H), 4.27-4.15 (m, 8H), 3.15 (s, 3H), 3.05-2.92 (m, 6H), 2.90 (d, J=5.9 Hz, 2H) ppm.

### Example Compound No. 8: N-((3-fluoropyridin-2-yl)methyl)-2-(2-((2-(1-(2-morpholinoethyl)-1H-benzo[d]imidazol-2-yl)ethyl)amino)ethyl)oxazolo[4,5-c]pyridin-4-amine

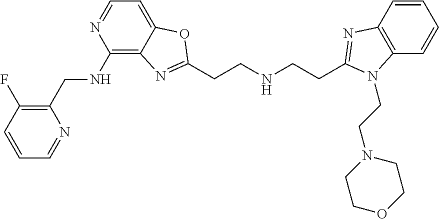 [SMILES: Fc1cccnc1CNc1nccc2oc(CCNCCc3nc4ccccc4n3CCN3CCOCC3)nc12]

2-(2-aminoethyl)-N-((3-fluoropyridin-2-yl)methyl)oxazolo[4,5-c]pyridin-4-amine (150 mg, 522 μmol, 1 eq.), 4-(2-(2-vinyl-1H-benzo[d]imidazol-1-yl)ethyl)morpholine (134 mg, 522 μmol, 1 eq.) and ammonium acetate (48.3 mg, 627 μmol, 1.2 eq.) were suspended in acetonitrile (5 mL). The reaction mixture was heated to 50° C. overnight. The reaction mixture was concentrated onto silica and then purified by flash column chromatography (CH₂Cl₂/MeOH 0-20% MeOH) obtaining N-((3-fluoropyridin-2-yl)methyl)-2-(2-((2-(1-(2-morpholinoethyl)-1H-benzo[d]imidazol-2-yl)ethyl)amino)ethyl)oxazolo[4,5-c]pyridin-4-amine (32.3 mg, 59.3 μmol, 11.4%) as a yellowish solid.

LCMS (ESI) m/z=545.1. ¹H NMR (400 MHz, DMSO) δ 8.34 (ddt, J=4.8, 3.1, 1.5 Hz, 1H), 7.86 (d, J=5.8 Hz, 1H), 7.67 (ddd, J=10.0, 8.3, 1.3 Hz, 1H), 7.49-7.43 (m, 2H), 7.36 (dt, J=8.5, 4.4 Hz, 1H), 7.17-7.07 (m, 3H), 6.89 (d, J=5.7 Hz, 1H), 4.83 (dd, J=5.6, 1.7 Hz, 2H), 4.25 (t, J=6.5 Hz, 2H), 3.49 (t, J=4.6 Hz, 4H), 3.11-3.04 (m, 6H), 3.04-2.98 (m, 2H), 2.56 (t, J=6.5 Hz, 2H), 2.38 (dd, J=5.6, 3.4 Hz, 4H) ppm.

### Example compound No. 9: N-((3-fluoropyridin-2-yl)methyl)-2-(2-((2-(1-(2-morpholinoethyl)-6,7-dihydro-1H-[1,4]dioxino[2′,3′:4,5]benzo[1,2-d]imidazol-2-yl)ethyl)amino)ethyl)oxazolo[4,5-c]pyridin-4-amine

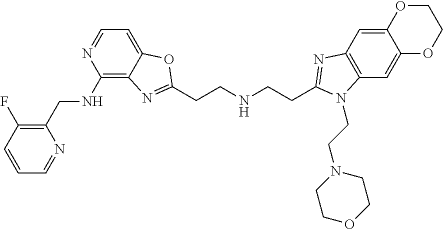 [SMILES: Fc1cccnc1CNc1nccc2oc(CCNCCc3nc4cc5c(cc4n3CCN3CCOCC3)OCCO5)nc12]

2-(1-(2-morpholinoethyl)-6,7-dihydro-1H-[1,4]dioxino[2′,3′:4,5]benzo[1,2-d]imidazol-2-yl)ethan-1-amine trihydrochloride (163 mg, 370 μmol, 1 eq.) and sodium hydroxide (207 mg, 30% Wt, 1.55 mmol, 4.2 eq.) were dissolved in water (3 mL). N-((3-fluoropyridin-2-yl)methyl)-2-vinyloxazolo[4,5-c]pyridin-4-amine (100 mg, 370 μmol, 1 eq.) was added and the reaction mixture was heated to 80° C. for 48 hour. The reaction mixture was allowed to cool down to 23° C. before the pH was adjusted to pH=7 using 3N hydrochloric acid. The reaction mixture was concentrated and purified by reverse phase column chromatography (H₂O/(CH₃CN/MeOH)+0.1% TFA, 95/%→5/95) followed by SCX-column (1-7N NH₃ in MeOH) affording the desired N-((3-fluoropyridin-2-yl)methyl)-2-(2-((2-(1-(2-morpholino-ethyl)-6,7-dihydro-1H-[1,4]dioxino[2′,3′:4,5]benzo[1,2-d]imidazol-2-yl)ethyl)amino)ethyl)-oxazolo[4,5-c]pyridin-4-amine (25.0 mg, 41.5 μmol, 11.2%) as a yellow solid.

LCMS (ESI) m/z=603.6. ¹H NMR (400 MHz, DMSO) δ 8.20 (dt, J=4.7, 1.5 Hz, 1H), 7.86 (d, J=5.4 Hz, 1H), 7.77-7.69 (m, 1H), 7.36 (s, 1H), 7.04-6.89 (m, 3H), 6.56 (d, J=5.5 Hz, 1H), 5.62 (d, J=1.6 Hz, 2H), 4.23-4.18 (m, 4H), 4.13 (t, J=6.7 Hz, 2H), 3.49 (t, J=4.6 Hz, 4H), 3.02-2.94 (m, 6H), 2.93-2.88 (m, 2H), 2.54-2.49 (m, 2H), 2.39-2.34 (m, 4H) ppm.

### Example Compound No. 10: 2-(2-((2-(1-benzyl-1H-benzo[d]imidazol-2-yl)ethyl)amino)ethyl)-N-((3-fluoropyridin-2-yl)methyl)oxazolo[4,5-c]pyridin-4-amine

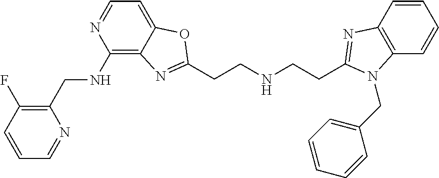 [SMILES: Fc1cccnc1CNc1nccc2oc(CCNCCc3nc4ccccc4n3Cc3ccccc3)nc12]

2-(2-aminoethyl)-N-((3-fluoropyridin-2-yl)methyl)oxazolo[4,5-c]pyridin-4-amine (100 mg, 348 μmol, 1 eq.), 1-benzyl-2-vinyl-1H-benzo[d]imidazole (92.0 mg, 348 μmol, 1 eq.) and ammonium acetate (32.2 mg, 418 μmol, 1.2 eq.) were suspended in acetonitrile (2.5 mL). The reaction mixture was heated to 50° C. overnight. The reaction mixture was concentrated onto silica and then purified by flash column chromatography (CH₂Cl₂/MeOH 0-20% MeOH) obtaining 2-(2-((2-(1-benzyl-1H-benzo[d]imidazol-2-yl)ethyl)amino)ethyl)-N-((3-fluoropyridin-2-yl)methyl)oxazolo[4,5-c]pyridin-4-amine (35.1 mg, 67.3 μmol, 19%) as a brownish oil.

LCMS (ESI) m/z=522.5. ¹H NMR (400 MHz, DMSO) δ 8.33 (dt, J=4.7, 1.5 Hz, 1H), 7.86 (d, J=5.7 Hz, 1H), 7.67 (ddd, J=10.0, 8.3, 1.3 Hz, 1H), 7.57-7.49 (m, 1H), 7.47-7.40 (m, 1H), 7.40-7.33 (m, 1H), 7.31-7.22 (m, 3H), 7.19-7.11 (m, 3H), 7.11-7.05 (m, 2H), 6.89 (d, J=5.8 Hz, 1H), 5.47 (s, 2H), 4.83 (dd, J=5.6, 1.7 Hz, 2H), 3.14-3.06 (m, 6H), 3.01 (t, J=6.4 Hz, 2H) ppm.

### Example Compound No. 11 N-((3-fluoropyridin-2-yl)methyl)-2-(2-((2-(1-(3-methoxybenzyl)-1H-benzo[d]-imidazol-2-yl)ethyl)amino)ethyl)oxazolo[4,5-c]pyridin-4-amine

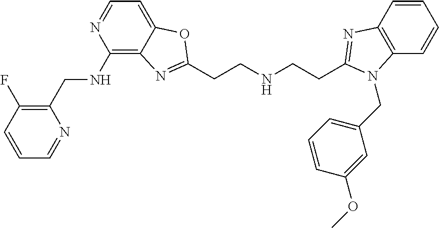 [SMILES: COc1cccc(Cn2c(CCNCCc3nc4c(NCc5ncccc5F)nccc4o3)nc3ccccc32)c1]

2-(2-aminoethyl)-N-((3-fluoropyridin-2-yl)methyl)oxazolo[4,5-c]pyridin-4-amine (100 mg, 348 μmol, 1 eq.), 1-(2-methoxybenzyl)-2-vinyl-1H-benzo[d]imidazole (81.6 mg, 348 μmol, 1 eq.) and ammonium acetate (32.2 mg, 418 μmol, 1.2 eq.) were suspended in acetonitrile (2.5 mL). The reaction mixture was heated to 50° C. overnight. The reaction mixture was concentrated onto silica and then purified by flash column chromatography (CH₂Cl₂/MeOH 0-20% MeOH) obtaining N-((3-fluoropyridin-2-yl)methyl)-2-(2-((2-(1-(2-methoxybenzyl)-1H-benzo[d]imidazol-2-yl)ethyl)amino)ethyl)oxazolo[4,5-c]pyridin-4-amine (52.2 mg, 92.8 μmol, 27%) as a brownish oil.

LCMS (ESI) m/z=552.6. ¹H NMR (400 MHz, DMSO) δ 8.33 (dt, J=4.7, 1.5 Hz, 1H), 7.86 (d, J=5.8 Hz, 1H), 7.67 (ddd, J=10.1, 8.3, 1.3 Hz, 1H), 7.55-7.49 (m, 1H), 7.46-7.40 (m, 1H), 7.36 (dt, J=8.6, 4.4 Hz, 1H), 7.22-7.09 (m, 4H), 6.89 (d, J=5.8 Hz, 1H), 6.81 (ddd, J=8.3, 2.7, 0.9 Hz, 1H), 6.67 (t, J=2.0 Hz, 1H), 6.60-6.55 (m, 1H), 5.43 (s, 2H), 4.83 (dd, J=5.6, 1.7 Hz, 2H), 3.66 (s, 3H), 3.11-3.04 (m, 6H), 3.00 (t, J=6.3 Hz, 2H) ppm.

### Example Compound No. 12: 2-(2-((2-(1-(3-chlorobenzyl)-1H-benzo[d]imidazol-2-yl)ethyl)amino)ethyl)-N-((3-fluoropyridin-2-yl)methyl)oxazolo[4,5-c]pyridin-4-amine

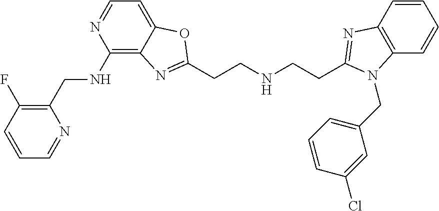 [SMILES: Fc1cccnc1CNc1nccc2oc(CCNCCc3nc4ccccc4n3Cc3cccc(Cl)c3)nc12]

2-(2-aminoethyl)-N-((3-fluoropyridin-2-yl)methyl)oxazolo[4,5-c]pyridin-4-amine (175 mg, 609 μmol, 1 eq.), 1-(3-chlorobenzyl)-2-vinyl-1H-benzo[d]imidazole (164 mg, 609 μmol, 1 eq.) and ammonium acetate (56.3 mg, 731 μmol, 1.2 eq.) were suspended in acetonitrile (5 mL). The reaction mixture was heated to 50° C. overnight. The reaction mixture was concentrated onto silica and then purified by flash column chromatography (CH₂Cl₂/MeOH 0-20% MeOH) obtaining 2-(2-((2-(1-(3-chlorobenzyl)-1H-benzo[d]imidazol-2-yl)ethyl)amino)ethyl)-N-((3-fluoropyridin-2-yl)methyl)oxazolo[4,5-c]pyridin-4-amine (65.1 mg, 117 μmol, 19.2%) as a brownish oil.

LCMS (ESI) m/z=557.2. ¹H NMR (400 MHz, DMSO) δ 8.33 (dt, J=4.7, 1.5 Hz, 1H), 7.87 (d, J=5.8 Hz, 1H), 7.67 (ddd, J=10.0, 8.3, 1.3 Hz, 1H), 7.56-7.51 (m, 1H), 7.49-7.40 (m, 1H), 7.40-7.33 (m, 1H), 7.34-7.30 (m, 2H), 7.22-7.09 (m, 4H), 7.04-6.96 (m, 1H), 6.90 (d, J=5.8 Hz, 1H), 5.50 (s, 2H), 4.84 (dd, J=5.6, 1.7 Hz, 2H), 3.22-3.11 (m, 6H), 3.05 (t, J=6.9 Hz, 2H) ppm.

### Example Compound No. 13: 2-(2-((2-(1-(cyclopropylmethyl)-6,7-dihydro-1H-[1,4]dioxino[2′,3′:4,5]benzo[1,2-d]-imidazol-2-yl)ethyl)amino)ethyl)-N-((3-fluoropyridin-2-yl)methyl)oxazolo[4,5-c]pyridin-4-amine

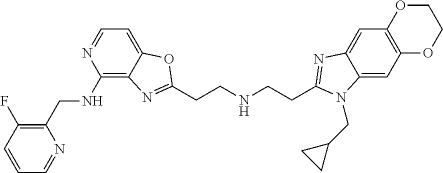 [SMILES: Fc1cccnc1CNc1nccc2oc(CCNCCc3nc4cc5c(cc4n3CC3CC3)OCCO5)nc12]

N-((3-fluoropyridin-2-yl)methyl)-2-(2-iodoethyl)oxazolo[4,5-c]pyridin-4-amine (125 mg, 314 μmol, 1 eq.) and 2-(1-(cyclopropylmethyl)-6,7-dihydro-1H-[1,4]dioxino[2′,3′:4,5]benzo[1,2-d]imidazol-2-yl)ethan-1-amine dihydrochloride (109 mg, 314 μmol, 1 eq.) were suspended in acetonitrile (2 mL). N,N-diisopropyl-ethylamine (130 mg, 175 μL, 1.00 mmol, 3.2 eq.) was added and the reaction mixture was heated to 60° C. for 48 hours. The reaction mixture was concentrated and then purified by reverse phase column chromatography (H₂O/(CH₃CN/MeOH)+0.1% TFA, 95/% a 5/95) followed by SCX-column (1-7N NH₃ in MeOH) affording the desired 2-(2-((2-(1-(cyclopropylmethyl)-6,7-dihydro-1H-[1,4]dioxino[2′,3′:4,5]-benzo[1,2-d]imidazol-2-yl)ethyl)amino)ethyl)-N-((3-fluoropyridin-2-yl)methyl)oxazolo[4,5-c]-pyridin-4-amine (33.4 mg, 61.4 μmol, 20%) as a yellow solid.

LCMS (ESI) m/z=544.6. ¹H NMR (400 MHz, DMSO) δ 8.33 (s, 1H), 7.85 (d, J=5.7 Hz, 1H), 7.67 (ddd, J=10.1, 8.3, 1.3 Hz, 1H), 7.36 (dt, J=8.6, 4.4 Hz, 1H), 7.14 (t, J=5.6 Hz, 1H), 7.03-6.96 (m, 2H), 6.91-6.86 (m, 2H), 4.83 (dd, J=5.6, 1.7 Hz, 2H), 4.20 (tt, J=5.3, 2.7 Hz, 4H), 3.96 (t, J=7.7 Hz, 2H), 3.07-3.03 (m, 4H), 2.99 (q, J=6.8 Hz, 2H), 2.91 (t, J=6.4 Hz, 2H), 1.17-1.07 (m, 1H), 0.43 (ddd, J=8.2, 6.0, 4.1 Hz, 2H), 0.34 (ddt, J=9.8, 6.4, 3.1 Hz, 2H) ppm.

### Example Compound No. 14: 2-(2-((2-(1-(2-(dimethylamino)ethyl)-6,7-dihydro-1H-[1,4]dioxino[2′,3′:4,5]benzo[1,2-d]imidazol-2-yl)ethyl)amino)ethyl)-N-((3-fluoropyridin-2-yl)methyl)oxazolo[4,5-c]pyridin-4-amine

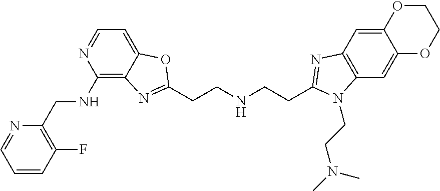 [SMILES: CN(C)CCn1c(CCNCCc2nc3c(NCc4ncccc4F)nccc3o2)nc2cc3c(cc21)OCCO3]

2-(2-(2-aminoethyl)-6,7-dihydro-1H-[1,4]dioxino[2′,3′:4,5]benzo[1,2-d]imidazol-1-yl)-N,N-dimethylethan-1-amine trihydrochloride (148 mg, 370 μmol, 1 eq.) and sodium hydroxide (30% Wt, 207 mg, 1.55 mmol, 4.2 eq.) were dissolved in water (4 mL). N-((3-fluoropyridin-2-yl)methyl)-2-vinyloxazolo[4,5-c]pyridin-4-amine (100 mg, 370 μmol, 1 eq.) was added and the reaction mixture was heated to 80° C. for 48 hour. The reaction mixture was allowed to cool down to r.t. before the pH was adjusted to pH=7 using 3N hydrochloric acid. The reaction mixture was concentrated and purified by flash column chromatography (CH₂Cl₂/MeOH, 0→20% MeOH) followed by SCX-column (1-7N NH₃ in MeOH) afforded the desired 2-(2-((2-(1-(2-(dimethylamino)ethyl)-6,7-dihydro-1H-[1,4]dioxino[2′,3′:4,5]benzo[1,2-d]imidazol-2-yl)ethyl)amino)ethyl)-N-((3-fluoropyridin-2-yl)methyl)oxazolo[4,5-c]pyridin-4-amine (65.0 mg, 116 μmol, 31%) as a pale yellow solid.

LCMS (ESI) m/z=561.7. ¹H NMR (400 MHz, DMSO) δ 8.20 (dd, J=4.7, 1.4 Hz, 1H), 7.81 (d, J=5.5 Hz, 1H), 7.72 (ddd, J=9.9, 8.3, 1.3 Hz, 1H), 7.36 (dt, J=8.6, 4.4 Hz, 1H), 6.93 (s, 1H), 6.92 (s, 1H), 6.52 (d, J=5.5 Hz, 1H), 5.63-5.55 (m, 2H), 4.25-4.15 (m, 4H), 4.10 (t, J=6.7 Hz, 2H), 2.97 (td, J=8.6, 3.5 Hz, 6H), 2.89 (t, J=6.4 Hz, 2H), 2.48-2.42 (m, 2H), 2.14 (s, 6H) ppm.

### Example Compound No. 15: N-((3-fluoropyridin-2-yl)methyl)-2-(2-((2-(1-(pyridin-2-ylmethyl)-1H-benzo[d]imidazol-2-yl)ethyl)amino)ethyl)oxazolo[4,5-c]pyridin-4-amine

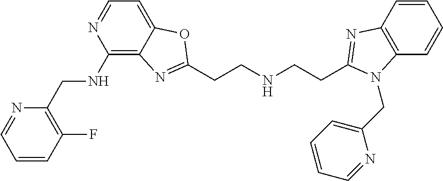 [SMILES: Fc1cccnc1CNc1nccc2oc(CCNCCc3nc4ccccc4n3Cc3ccccn3)nc12]

2-(1-(pyridin-2-ylmethyl)-1H-benzo[d]imidazol-2-yl)ethan-1-amine (93.4 mg, 370 μmol, 1 eq.) and sodium hydroxide (30% Wt, 59.2 mg, 444 μmol, 1.2 eq.) were dissolved in water (4 mL). N-((3-fluoropyridin-2-yl)methyl)-2-vinyloxazolo[4,5-c]pyridin-4-amine (100 mg, 370 μmol, 1 eq.) was added and the reaction mixture was heated to 80° C. for 48 hour. The reaction mixture was allowed to cool down to r.t. before the pH was adjusted to pH=7 using 3N hydrochloric acid. The reaction mixture was concentrated and purified by flash column chromatography (CH₂Cl₂/MeOH, 0→20% MeOH) followed by SCX-columns (1-7N NH₃ in MeOH) afforded the desired N-((3-fluoropyridin-2-yl)methyl)-2-(2-((2-(1-(pyridin-2-ylmethyl)-1H-benzo[d]imidazol-2-yl)ethyl)amino)ethyl)oxazolo[4,5-c]pyridin-4-amine (32 mg, 61 μmol, 17%) as a pale yellow solid.

LCMS (ESI) m/z=523.5. ¹H NMR (400 MHz, DMSO) δ 8.47 (dd, J=5.0, 1.7 Hz, 1H), 8.20 (d, J=4.7 Hz, 1H), 7.93 (d, J=5.5 Hz, 1H), 7.81-7.71 (m, 2H), 7.51 (td, J=6.2, 2.4 Hz, 2H), 7.38 (dt, J=8.6, 4.4 Hz, 1H), 7.33 (d, J=7.8 Hz, 1H), 7.28 (dd, J=7.6, 4.9 Hz, 1H), 7.15 (tt, J=7.3, 5.6 Hz, 2H), 6.65 (d, J=5.5 Hz, 1H), 5.67 (s, 2H), 5.58 (s, 2H), 3.57-3.50 (m, 4H), 3.38 (t, J=7.0 Hz, 2H), 3.30 (t, J=6.9 Hz, 2H) ppm.

### Example Compound No. 16: -((3-fluoropyridin-2-yl)methyl)-2-(2-((2-(1-phenethyl-1H-benzo[d]imidazol-2-yl)ethyl)amino)ethyl)oxazolo[4,5-c]pyridin-4-amine

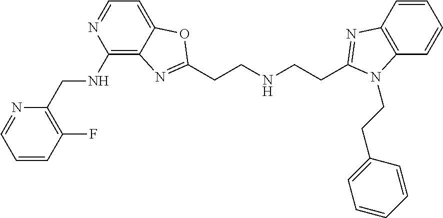 [SMILES: Fc1cccnc1CNc1nccc2oc(CCNCCc3nc4ccccc4n3CCc3ccccc3)nc12]

2-(1-phenethyl-1H-benzo[d]imidazol-2-yl)ethan-1-amine hydrochloride (112 mg, 370 μmol, 1 eq.) and sodium hydroxide (30% Wt, 109 mg, 814 μmol, 2.2 eq.) were dissolved in water (4 mL). N-((3-fluoropyridin-2-yl)methyl)-2-vinyloxazolo[4,5-c]pyridin-4-amine (100 mg, 370 μmol, 1 eq.) was added and the reaction mixture was heated to 80° C. for 48 hour. The reaction mixture was allowed to cool down to r.t. before the pH was adjusted to pH=7 using 3N hydrochloric acid. The reaction mixture was concentrated and purified by flash column chromatography (CH₂Cl₂/MeOH, 0→20% MeOH) followed by SCX-columns (1-7N NH₃ in MeOH) afforded the desired N-((3-fluoropyridin-2-yl)methyl)-2-(2-((2-(1-phenethyl-1H-benzo[d]imidazol-2-yl)ethyl)amino)ethyl)oxazolo[4,5-c]pyridin-4-amine (68.2 mg, 127 μmol, 34%) as a pale yellow solid.

LCMS (ESI) m/z=536.7. ¹H NMR (400 MHz, DMSO) δ 8.33 (dt, J=4.7, 1.5 Hz, 1H), 7.86 (d, J=5.8 Hz, 1H), 7.67 (ddd, J=10.1, 8.3, 1.4 Hz, 1H), 7.47 (ddd, J=7.0, 4.8, 1.4 Hz, 2H), 7.36 (dt, J=8.6, 4.5 Hz, 1H), 7.24-7.09 (m, 6H), 7.07-7.04 (m, 2H), 6.89 (d, J=5.8 Hz, 1H), 4.83 (dd, J=5.7, 1.7 Hz, 2H), 4.36 (t, J=7.2 Hz, 2H), 3.07-2.94 (m, 6H), 2.87 (t, J=7.0 Hz, 2H), 2.65 (t, J=6.9 Hz, 2H) ppm.

### Example Compound No. 17: N-((3-fluoropyridin-2-yl)methyl)-2-(2-((2-(1-phenyl-1H-benzo[d]imidazol-2-yl)ethyl)amino)ethyl)oxazolo[4,5-c]pyridin-4-amine

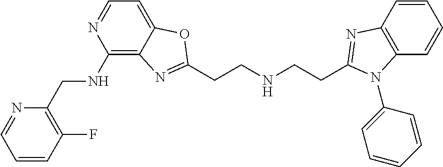 [SMILES: Fc1cccnc1CNc1nccc2oc(CCNCCc3nc4ccccc4n3-c3ccccc3)nc12]

2-(1-phenyl-1H-benzo[d]imidazol-2-yl)ethan-1-amine dihydrochloride (115 mg, 370 μmol, 1 eq.) and sodium hydroxide (30% Wt, 158 mg, 1.18 mmol, 3.2 eq.) were dissolved in water (4 mL). N-((3-fluoropyridin-2-yl)methyl)-2-vinyloxazolo[4,5-c]pyridin-4-amine (100 mg, 370 μmol, 1 eq.) was added and the reaction mixture was heated to 80° C. for 48 hour.

The reaction mixture was allowed to cool down to r.t. before the pH was adjusted to pH=7 using 3N hydrochloric acid. The reaction mixture was concentrated and purified by flash column chromatography (CH₂Cl₂/MeOH, 0→20% MeOH) followed by SCX-columns (1-7N NH₃ in MeOH) afforded the desired N-((3-fluoropyridin-2-yl)methyl)-2-(2-((2-(1-phenyl-1H-benzo[d]imidazol-2-yl)ethyl)-amino)ethyl)oxazolo[4,5-c]pyridin-4-amine (53.2 mg, 105 μmol, 28%) as a pale yellow solid.

LCMS (ESI) m/z=508.5 ppm. ¹H NMR (400 MHz, DMSO) δ 8.33 (dt, J=4.7, 1.5 Hz, 1H), 7.67 (ddd, J=10.0, 8.3, 1.3 Hz, 1H), 7.63-7.57 (m, 3H), 7.56-7.48 (m, 3H), 7.36 (dt, J=8.6, 4.4 Hz, 1H), 7.22-7.12 (m, 3H), 7.08-7.03 (m, 1H), 6.87 (d, J=5.8 Hz, 1H), 4.82 (dd, J=5.6, 1.7 Hz, 2H), 3.04-2.90 (m, 6H), 2.84 (t, J=6.6 Hz, 2H) ppm.

### Example Compound No. 18: N-((3-fluoropyridin-2-yl)methyl)-2-(2-((2-(1-(2-(4-methylpiperazin-1-yl)ethyl)-6,7-dihydro-1H-[1,4]dioxino[2′,3′:4,5]benzo[1,2-d]imidazol-2-yl)ethyl)amino)ethyl)oxazolo[4,5-c]pyridin-4-amine

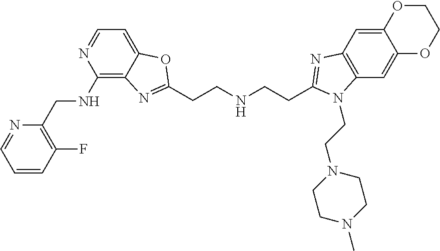 [SMILES: CN1CCN(CCn2c(CCNCCc3nc4c(NCc5ncccc5F)nccc4o3)nc3cc4c(cc32)OCCO4)CC1]

2-(1-(2-(4-methylpiperazin-1-yl)ethyl)-6,7-dihydro-1H-[1,4]dioxino[2′,3′:4,5]benzo[1,2-d]imidazol-2-yl)ethan-1-amine trihydrochloride (168 mg, 370 μmol, 1 eq.) and sodium hydroxide (30% Wt, 207 mg, 1.55 mmol, 4.2 eq.) were dissolved in water (4 mL). N-((3-fluoropyridin-2-yl)methyl)-2-vinyloxazolo[4,5-c]pyridin-4-amine (100 mg, 370 μmol, 1 eq.) was added and the reaction mixture was heated to 80° C. for 48 hour. The reaction mixture was allowed to cool down to r.t. before the pH was adjusted to pH=7 using 3N hydrochloric acid. The reaction mixture was concentrated and purified by flash column chromatography (CH₂Cl₂/MeOH, 0→20% MeOH) followed by SCX-columns (1-7N NH₃ in MeOH) affording the desired N-((3-fluoropyridin-2-yl)methyl)-2-(2-((2-(1-(2-(4-methylpiperazin-1-yl)ethyl)-6,7-dihydro-1H-[1,4]dioxino[2′,3′:4,5]benzo[1,2-d]imidazol-2-yl)ethyl)amino)ethyl)oxazolo[4,5-c]pyridin-4-amine (48.1 mg, 78.1 μmol, 21%) as a yellowish solid.

LCMS (ESI) m/z=616.8 ppm. ¹H NMR (400 MHz, DMSO) δ 8.21 (dt, J=4.7, 1.5 Hz, 1H), 7.78-7.67 (m, 2H), 7.36 (dt, J=8.7, 4.4 Hz, 1H), 6.94-6.93 (m, 2H), 6.44 (d, J=5.6 Hz, 1H), 5.59 (s, 2H), 4.23-4.17 (m, 4H), 4.11 (t, J=6.5 Hz, 2H), 2.94 (ddt, J=22.5, 14.3, 7.0 Hz, 8H), 2.43-2.31 (m, 4H), 2.27-2.18 (m, 4H), 2.07 (s, 3H) ppm. —CH₂— covered by DMSO solvent signal.

### Example Compound No. 19: 2-(2-((2-(1-ethyl-1H-benzo[d]imidazol-2-yl)ethyl)amino)ethyl)-N-((3-fluoropyridin-2-yl)methyl)oxazolo[4,5-c]pyridin-4-amine

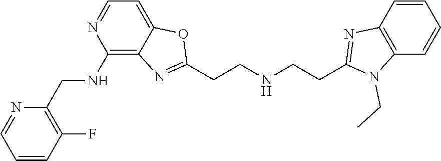 [SMILES: CCn1c(CCNCCc2nc3c(NCc4ncccc4F)nccc3o2)nc2ccccc21]

2-(1-ethyl-1H-benzo[d]imidazol-2-yl)ethan-1-amine dihydrochloride (97.0 mg, 370 μmol, 1 eq.) and sodium hydroxide (30% Wt, 158 mg, 1.18 mmol, 3.2 eq.) were dissolved in water (4 mL). N-((3-fluoropyridin-2-yl)methyl)-2-vinyloxazolo[4,5-c]pyridin-4-amine (100 mg, 370 μmol, 1 eq.) was added and the reaction mixture was heated to 80° C. for 48 hour.

The reaction mixture was allowed to cool down to r.t. before the pH was adjusted to pH=7 using 3N hydrochloric acid. The reaction mixture was concentrated and purified by flash column chromatography (CH₂Cl₂/MeOH, 0→20% MeOH) followed by SCX-columns (1-7N NH₃ in MeOH) affording the desired 2-(2-((2-(1-ethyl-1H-benzo[d]imidazol-2-yl)ethyl)amino)ethyl)-N-((3-fluoropyridin-2-yl)methyl)oxazolo[4,5-c]pyridin-4-amine (74.0 mg, 161 μmol, 44%) as an off-white solid.

LCMS (ESI) m/z=460.5. ¹H NMR (400 MHz, DMSO) δ 8.34 (dt, J=4.7, 1.5 Hz, 1H), 7.89 (d, J=5.8 Hz, 1H), 7.68 (ddd, J=10.0, 8.3, 1.3 Hz, 1H), 7.50 (t, J=8.2 Hz, 2H), 7.37 (dt, J=8.6, 4.4 Hz, 1H), 7.21-7.15 (m, 2H), 7.12 (td, J=7.6, 1.2 Hz, 1H), 6.93 (d, J=5.8 Hz, 1H), 4.84 (dd, J=5.6, 1.8 Hz, 2H), 4.22 (q, J=7.2 Hz, 2H), 3.41-3.25 (m, 8H), 3.17 (t, J=7.0 Hz, 2H), 1.28 (t, J=7.2 Hz, 3H) ppm.

### Example Compound No. 20: 2-(2-((2-(1-(2-(dimethylamino)ethyl)-1H-benzo[d]imidazol-2-yl)ethyl)amino)ethyl)-N-((3-fluoropyridin-2-yl)methyl)oxazolo[4,5-c]pyridin-4-amine

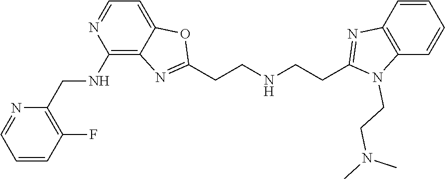 [SMILES: CN(C)CCn1c(CCNCCc2nc3c(NCc4ncccc4F)nccc3o2)nc2ccccc21]

2-(2-(2-aminoethyl)-1H-benzo[d]imidazol-1-yl)-N,N-dimethylethan-1-amine trihydrochloride (240 mg, 703 μmol, 1 eq.) and sodium hydroxide (30% Wt, 394 mg, 2.95 mmol, 4.2 eq.) were dissolved in water (4 mL). N-((3-fluoropyridin-2-yl)methyl)-2-vinyloxazolo[4,5-c]pyridin-4-amine (190 mg, 703 μmol, 1 eq.) was added and the reaction mixture was heated to 80° C. for 48 hour. The reaction mixture was allowed to cool down to r.t. before the pH was adjusted to pH=7 using 3N hydrochloric acid. The reaction mixture was concentrated and purified by RP18 flash column chromatography (water/methanol+0.1% NH₃, 5%→95% methanol) afforded the desired 2-(2-((2-(1-(2-(dimethylamino)ethyl)-1H-benzo[d]imidazol-2-yl)ethyl)amino)ethyl)-N-((3-fluoropyridin-2-yl)methyl)oxazolo[4,5-c]pyridin-4-amine (34.0 mg, 67.6 μmol, 10%) as a pale yellow solid.

LCMS (ESI) m/z=503.2. ¹H NMR (400 MHz, DMSO) δ 8.16 (d, J=4.6 Hz, 1H), 7.88 (d, J=5.5 Hz, 1H), 7.73 (t, J=9.3 Hz, 1H), 7.54 (dd, J=18.3, 7.8 Hz, 2H), 7.34 (dt, J=8.7, 4.4 Hz, 1H), 7.19 (dt, J=20.8, 7.3 Hz, 2H), 6.60 (d, J=5.5 Hz, 1H), 5.64 (s, 2H), 4.35 (t, J=6.7 Hz, 2H), 4.08 (s, 2H), 3.05 (p, J=5.4 Hz, 4H), 2.61 (t, J=6.6 Hz, 2H), 2.19 (s, 6H) ppm.

### Example Compound No. 21: 2-(2-((2-(1-ethyl-6,7-dihydro-1H-[1,4]dioxino[2′,3′:4,5]benzo[1,2-d]imidazol-2-yl)ethyl)amino)ethyl)-N-((3-fluoropyridin-2-yl)methyl)oxazolo[4,5-c]pyridin-4-amine formate

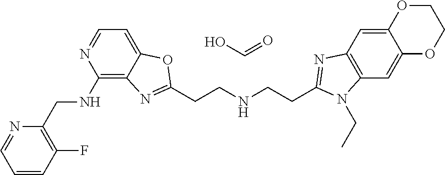

2-(1-ethyl-6,7-dihydro-1H-[1,4]dioxino[2′,3′:4,5]benzo[1,2-d]imidazol-2-yl)ethan-1-amine dihydrochloride (118 mg, 370 μmol, 1 eq.) and sodium hydroxide (30% Wt, 158 mg, 1.18 mmol, 3.2 eq.) were dissolved in water (4 mL). N-((3-fluoropyridin-2-yl)methyl)-2-vinyloxazolo[4,5-c]pyridin-4-amine (100 mg, 370 μmol, 1 eq.) was added and the reaction mixture was heated to 80° C. for 48 hour. The reaction mixture was allowed to cool down to r.t. before the pH was adjusted to pH=7 using 3N hydrochloric acid. The reaction mixture was concentrated and purified by preparative RP18 HPLC (water/acetonitrile/methanol+0.1% formic acid, 5→95% acetonitrile/methanol) affording the desired 2-(2-((2-(1-ethyl-6,7-dihydro-1H-[1,4]dioxino[2′,3′:4,5]benzo[1,2-d]imidazol-2-yl)ethyl)amino)ethyl)-N-((3-fluoropyridin-2-yl)-methyl)oxazolo[4,5-c]pyridin-4-amine formate (23.6 mg, 45.6 μmol, 12%) as a white solid.

LCMS(ESI) m/z=536.6. ¹H NMR (400 MHz, DMSO) δ 8.34 (dt, J=4.7, 1.5 Hz, 1H), 8.20 (s, 1H), 7.86 (d, J=5.8 Hz, 1H), 7.67 (ddd, J=10.1, 8.3, 1.3 Hz, 1H), 7.36 (dt, J=8.6, 4.4 Hz, 1H), 6.99-6.87 (m, 3H), 4.83 (dd, J=5.6, 1.7 Hz, 2H), 4.20 (tq, J=6.9, 3.0 Hz, 4H), 4.08 (q, J=7.1 Hz, 2H), 3.05 (t, J=6.9 Hz, 2H), 2.93 (t, J=6.9 Hz, 2H), 1.21 (t, J=7.1 Hz, 3H) ppm.

### Example Compound No. 22: N-((3-fluoropyridin-2-yl)methyl)-2-(2-((2-(1-methyl-1H-benzo[d]imidazol-2-yl)ethyl)amino)ethyl)oxazolo[4,5-c]pyridin-4-amine formate

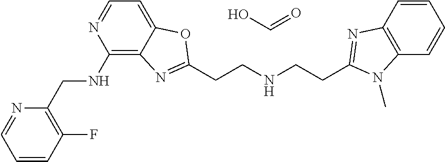

2-(1-methyl-1H-benzo[d]imidazol-2-yl)ethan-1-amine dihydrochloride (91.8 mg, 370 μmol, 1 eq.) and sodium hydroxide (30% Wt, 158 mg, 1.18 mmol, 3.2 eq.) were dissolved in water (4 mL). N-((3-fluoropyridin-2-yl)methyl)-2-vinyloxazolo[4,5-c]pyridin-4-amine (100 mg, 370 μmol, 1 eq.) was added and the reaction mixture was heated to 80° C. for 48 hour. The reaction mixture was allowed to cool down to r.t. before the pH was adjusted to pH=7 using 3N hydrochloric acid. The reaction mixture was concentrated and purified by preparative RP18 HPLC (water/acetonitrile/methanol+0.1% formic acid, 5→95% acetonitrile/methanol) affording the desired N-((3-fluoropyridin-2-yl)methyl)-2-(2-((2-(1-methyl-1H-benzo[d]imidazol-2-yl)ethyl)amino)ethyl)oxazolo[4,5-c]pyridin-4-amine formate (38.8 mg, 45.6 μmol, 24%) as a white solid.

LCMS(ESI) m/z=446.7. ¹H NMR (400 MHz, DMSO) δ 8.34 (dt, J=4.7, 1.5 Hz, 1H), 8.20 (s, 1H), 7.86 (d, J=5.7 Hz, 1H), 7.67 (ddd, J=10.0, 8.4, 1.3 Hz, 1H), 7.50-7.41 (m, 2H), 7.36 (dt, J=8.6, 4.4 Hz, 1H), 7.16 (ddd, J=7.9, 5.2, 1.3 Hz, 2H), 7.10 (td, J=7.5, 1.3 Hz, 1H), 6.90 (d, J=5.7 Hz, 1H), 4.83 (dd, J=5.6, 1.8 Hz, 2H), 3.71 (s, 3H), 3.16-2.96 (m, 8H) ppm.

### Example Compound No. 23: N-((3-fluoropyridin-2-yl)methyl)-2-(2-((2-(1-phenyl-6,7-dihydro-1H-[1,4]dioxino[2′,3′:4,5]benzo[1,2-d]imidazol-2-yl)ethyl)amino)ethyl)oxazolo[4,5-c]pyridin-4-amine formate

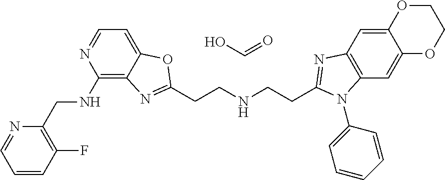

2-(1-phenyl-6,7-dihydro-1H-[1,4]dioxino[2′,3′:4,5]benzo[1,2-d]imidazol-2-yl)ethan-1-amine dihydrochloride (136 mg, 370 μmol, 1 eq.) and sodium hydroxide (30% Wt, 158 mg, 1.18 mmol, 3.2 eq.) were dissolved in water (4 mL). N-((3-fluoropyridin-2-yl)methyl)-2-vinyloxazolo[4,5-c]pyridin-4-amine (100 mg, 370 μmol, 1 eq.) was added and the reaction mixture was heated to 80° C. for 48 hour. The reaction mixture was allowed to cool down to r.t. before the pH was adjusted to pH=7 using 3N hydrochloric acid. The reaction mixture was concentrated and purified by preparative RP18 HPLC (water/acetonitrile/methanol+0.1% formic acid, 5→95% acetonitrile/methanol) affording the desired N-((3-fluoropyridin-2-yl)methyl)-2-(2-((2-(1-phenyl-6,7-dihydro-1H-[1,4]dioxino[2′,3′:4,5]benzo[1,2-d]imidazol-2-yl)ethyl)amino)ethyl)oxazolo[4,5-c]pyridin-4-amine formate (40.2 mg, 71.1 μmol, 19%) as a white solid.

LCMS(ESI) m/z=566.8. ¹H NMR (400 MHz, DMSO) δ 8.33 (dt, J=4.7, 1.5 Hz, 1H), 8.18 (s, 1H), 7.85 (d, J=5.8 Hz, 1H), 7.67 (ddd, J=10.1, 8.3, 1.3 Hz, 1H), 7.58 (dd, J=8.3, 6.7 Hz, 2H), 7.54-7.48 (m, 1H), 7.47-7.43 (m, 2H), 7.36 (dt, J=8.6, 4.4 Hz, 1H), 7.13 (t, J=5.6 Hz, 1H), 7.03 (s, 1H), 6.88 (d, J=5.7 Hz, 1H), 6.44 (s, 1H), 4.82 (dd, J=5.6, 1.7 Hz, 2H), 4.33-4.19 (m, 4H), 3.03-2.89 (m, 6H), 2.79 (t, J=7.0 Hz, 2H) ppm.

1. A compound according to formula (I-A) 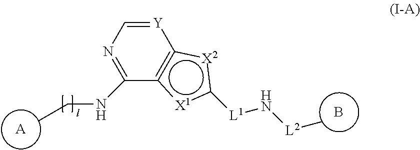
wherein
I is an integer selected from 1 and 2;
L¹ and L² each represent a linker group comprising 1 to 7 carbon atoms and which are independently selected from:
a linear C₁-C₃-alkyl group —[CH₂]ₘ— or —[CH₂]ₙ—, respectively, wherein m and n are independently an integer selected from 1, 2 and 3,
a branched C₁-C₄-alkyl group, and
a C3-C6-cycloalkyl group, which forms a ring together with the nitrogen atom to which it is bonded;
X¹ is N, S or O; and
X² is N, S, or O; with the proviso that one of X¹ and X² is N;
Y is N or CR⁵ wherein R⁵ represents:
H,
halogen,
linear or branched C₁-C₃-alkyl, or
linear or branched C₁-C₃-haloalkyl;
A represents a group (a-1) 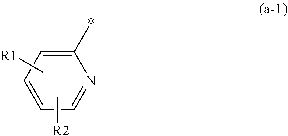
wherein * indicates the binding position;
R¹ and R² independently represent:
hydrogen
halogen,
linear or branched C₁-C₃-alkyl,
linear or branched C₁-C₃-haloalkyl, or
linear or branched C₁-C₃-alkoxy;
B represents one of the following groups (b-1), (b-2) and (b-3); 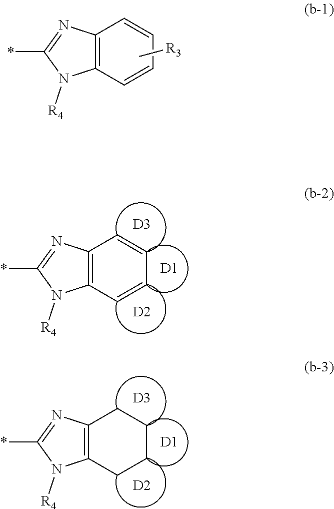
wherein * indicates the binding position;
R³ represents 0, 1, 2 or 3 substituents independently selected from:
linear or branched C₁-C₃-alkyl,
linear or branched C₁-C₃-haloalkyl,
linear or branched C₁-C₃-alkoxy,
unsubstituted or substituted 6-membered aryl,
unsubstituted or substituted 5- or 6-membered heteroaryl,
unsubstituted or substituted bicyclic heteroaryl,
unsubstituted or substituted 3- to 6-membered cycloalkyl,
unsubstituted or substituted 5- or 6-membered heterocyclyl,
unsubstituted or substituted 5- or 6-membered heterocyclylalkyl,
unsubstituted or substituted 6-membered arylalkinyl, or
unsubstituted or substituted 5- or 6-membered heteroarylalkinyl,
wherein a substituted aryl, heteroaryl, bicyclic heteroaryl, cycloalkyl, heterocyclyl, heterocyclylalkyl, arylalkinyl and heteroarylalkinyl group can optionally be substituted with 1, 2 or 3 substituents independently selected from:
halogen,
C₁-C₃-alkyl,
C₁-C₃-haloalkyl, and
C₁-C₃-alkoxy;
R⁴ represents;
hydrogen
unsubstituted or substituted linear or branched C₁-C₆-alkyl,
a dialkylether group [R(CH₂)—O—CH₂)_{y}—] wherein R⁶ represents a C₁-C₃-alkoxy group and x and y independently represents an integer selected from 1, 2 and 3,
unsubstituted or substituted 3- to 6-membered cycloalkyl,
unsubstituted or substituted 5- or 6-membered heterocyclyl,
unsubstituted or substituted 6-membered aryl, or
unsubstituted or substituted 5- or 6-membered heteroaryl,
wherein a substituted alkyl, cycloalkyl, heterocyclyl, aryl and heteroaryl group can optionally be substituted with 1 or 2 substituents, independently selected from;
halogen,
C₁-C₃-alkoxy,
C₆-cycloalkyloxy,
carboxyl,
aminocarbonyl,
mono- or di-alkylaminocarbonyl,
an amino group comprising —NH₂, mono- and dialkylamino,
unsubstituted or substituted 3- to 6-membered cycloalkyl,
unsubstituted or substituted 5- or 6-membered heterocyclyl,
unsubstituted or substituted 6-membered aryl,
unsubstituted or substituted 5- or 6-membered heteroaryl, and
unsubstituted or substituted bicyclic heteroaryl,
wherein a substituted cycloalkyl, heterocyclyl, aryl, heteroaryl and bicyclic heteroaryl group can optionally be substituted with 1, 2 or 3 substituents independently selected from:
hydroxy,
cyano,
halogen,
C₁-C₃-alkyl,
C₁-C₃-haloalkyl,
C₁-C₃-alkoxy,
carboxyl,
an amino (—NH₂) or mono- or di-alkylamino group,
aminocarbonyl, and
mono- or di-alkylaminocarbonyl,
wherein a monoalkylamino group and a monoalkylaminocarbonyl group may carry a further substituent on the mono-alkyl-chain, selected from:
C₁-C₃-alkoxy,
unsubstituted or substituted 6-membered aryl, and
unsubstituted or substituted 5- or 6-membered heteroaryl,
wherein a substituted aryl or heteroaryl group as a substituent of the mono-alkyl-chain can optionally be substituted with 1, 2 or 3 substituents independently selected from halogen, C₁-C₃-alkyl and C₁-C₃-haloalkyl;
with the proviso that
in formulae (b-1) R⁴ does not represent hydrogen when R³ is absent or when R³ is methyl; and
in formulae (b-2) and (b-3) one of D1, D2 and D3 is present and represents:
a fused 6-membered aryl ring,
a fused 5- or 6-membered heteroaryl ring,
a fused 5- or 6-membered cycloalkyl ring, or
a fused 5- or 6-membered heterocyclyl ring;
and the groups (b-2) and (b-3) carry 0, 1, 2 or 3 substituents, which are independently selected from:
halogen,
linear or branched C₁-C₃-alkyl,
linear or branched C₁-C₃-haloalkyl,
linear or branched C₁-C₃-alkoxy;
or a pharmaceutically acceptable salt thereof.

2. The compound or pharmaceutically acceptable salt thereof according to claim 1, with the further proviso that formula (b-1) R⁴ does not represent hydrogen when R³ is linear or branched C₁-C₃-alkyl, linear or branched C₁-C₃-haloalkyl, or linear or branched C₁-C₃-alkoxy.

3. The compound or pharmaceutically acceptable salt thereof according to claim 1, wherein R⁴ is not hydrogen.

4. The compound or pharmaceutically acceptable salt thereof according to claim 1, selected from compounds according to formula (I-B) 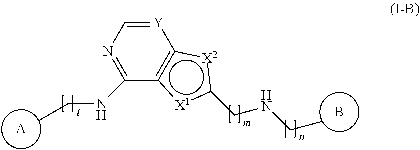
wherein I is an integer selected from 1 and 2; and m and n are independently an integer selected from 1, 2 and 3.

5. The compound or pharmaceutically acceptable salt thereof according to claim 1, wherein
R⁴ represents;
linear or branched C₁-C₆-alkyl,
a dialkylether group [R⁶(CH₂)ₓ—O—CH₂)_{y}—] wherein R⁶ represents a C₁-C₃-alkoxy group and x and y independently represent an integer from 1, 2 and 3,
3- to 6-membered cycloalkyl,
5- or 6-membered heterocyclyl, or
6-membered aryl,
wherein alkyl, cycloalkyl, heterocyclyl and aryl can optionally be substituted with 1 or 2 substituents, independently selected from:
C₁-C₃-alkoxy,
carboxyl,
aminocarbonyl,
mono- or di-alkylaminocarbonyl,
an amino group comprising —NH₂, mono- and dialkylamino
3- to 6-membered cycloalkyl, and
5- or 6-membered heterocyclyl,
unsubstituted or substituted 6-membered aryl,
unsubstituted or substituted 5- or 6-membered heteroaryl, and
unsubstituted or substituted bicyclic heteroaryl,
wherein a substituted aryl, heteroaryl and bicyclic heteroaryl group can optionally be substituted with 1, 2 or 3 substituents independently selected from;
hydroxy,
cyano,
halogen,
C₁-C₃-alkyl,
C₁-C₃-haloalkyl,
C₁-C₃-alkoxy,
carboxyl,
an amino (—NH₂) or mono- or di-alkylaminogroup,
aminocarbonyl, and
mono- or di-alkylaminocarbonyl,
wherein a monoalkylamino group and a monoalkylaminocarbonyl group may carry a further substituent on the mono-alkyl-chain, selected from:
C₁-C₃-alkoxy,
unsubstituted or substituted 6-membered aryl, and
unsubstituted or substituted 5- or 6-membered heteroaryl,
wherein a substituted aryl or heteroaryl group as a substituent of the mono-alkyl-chain can optionally be substituted with 1, 2 or 3 substituents independently selected from halogen, C₁-C₃-alkyl and C₁-C₃-haloalkyl.

6. The compound or pharmaceutically acceptable salt thereof according to claim 1, wherein
A represents a group (a-1) 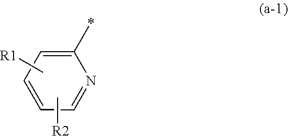
wherein * indicates the binding position;
R¹ and R² independently represent;
hydrogen,
halogen,
linear or branched C₁-C₃-alkyl,
linear or branched C₁-C₃-haloalkyl, or
linear or branched C₁-C₃-alkoxy;
B represents one of the following groups (b-1), (b-2) and (b-3): 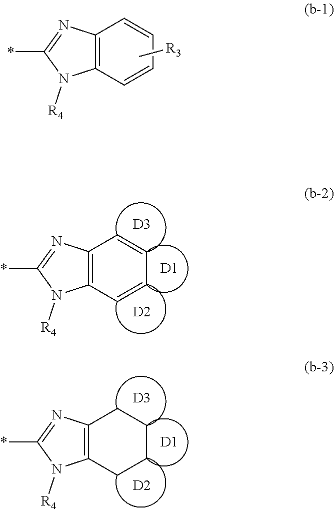
wherein * indicates the binding position;
R³ represents 0, 1, 2 or 3 substituents independently selected from:
linear or branched C₁-C₃-alkyl,
linear or branched C₁-C₃-haloalkyl,
linear or branched C₁-C₃-alkoxy,
unsubstituted or substituted 6-membered aryl,
unsubstituted or substituted 5- or 6-membered heteroaryl,
unsubstituted or substituted bicyclic heteroaryl,
3- to 6-membered cycloalkyl,
5- or 6-membered heterocyclyl,
5- or 6-membered heterocyclylalkyl, and
6-membered arylalkinyl
wherein a substituted aryl, heteroaryl and bicyclic heteroaryl group can optionally be substituted with 1, 2 or 3 substituents independently selected from:
halogen,
C₁-C₃-alkyl,
C₁-C₃-haloalkyl, and
C₁-C₃-alkoxy;
R⁴ represents:
linear or branched C₁-C₆-alkyl,
a dialkylether group [R⁶(CH₂)ₓ—O—CH₂)_{y}—] wherein R⁶ represents a C₁-C₃-alkoxy group and x and y independently represent an integer selected from 1, 2 and 3,
3- to 6-membered cycloalkyl, or
5- or 6-membered heterocyclyl,
6-membered aryl,
wherein alkyl, cycloalkyl, heterocyclyl and aryl can optionally be substituted with 1 or 2 substituents, independently selected from;
C₁-C₃-alkoxy,
carboxyl,
aminocarbonyl,
mono- or di-alkylaminocarbonyl,
an amino group comprising —NH2, mono- and dialkylamino
3- to 6-membered cycloalkyl, and
5- or 6-membered heterocyclyl,
unsubstituted or substituted 6-membered aryl,
unsubstituted or substituted 5- or 6-membered heteroaryl, and
unsubstituted or substituted bicyclic heteroaryl,
wherein a substituted aryl, heteroaryl and bicyclic heteroaryl group can optionally be substituted with 1, 2 or 3 substituents independently selected from:
hydroxy,
cyano,
halogen,
C₁-C₃-alkyl,
C₁-C₃-haloalkyl,
C₁-C₃-alkoxy,
carboxyl,
an amino (—NH₂) or mono- or di-alkylaminogroup,
aminocarbonyl, and
mono- or di-alkylaminocarbonyl,
wherein a monoalkylamino group and a monoalkylaminocarbonyl group may carry a further substituent on the mono-alkyl-chain, selected from:
C₁-C₃-alkoxy,
unsubstituted or substituted 6-membered aryl, and
unsubstituted or substituted 5- or 6-membered heteroaryl, wherein a substituted aryl or heteroaryl group as a substituent of the mono-alkyl-chain can optionally be substituted with 1, 2 or 3 substituents independently selected from halogen, C₁-C₃-alkyl and C₁-C₃-haloalkyl;
and in formulae (b-2) and (b-3) one of D1, D2 and D3 is present and represents
a fused 6-membered aryl ring,
a fused 5- or 6-membered heteroaryl ring,
a fused 5- or 6-membered cycloalkyl ring, or
a fused 5- or 6-membered heterocyclyl ring;
and the groups (b-2) and (b-3) carry 0, 1, 2 or 3 substituents, which are independently selected from:
halogen,
linear or branched C₁-C₃-alkyl,
linear or branched C₁-C₃-haloalkyl,
linear or branched C₁-C₃-alkoxy.

7. The compound or pharmaceutically acceptable salt thereof according to claim 1, wherein
A represents a group (a-1) 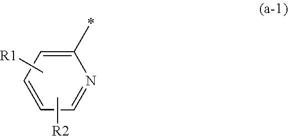
wherein * indicates the binding position;
R¹ and R² independently represent;
hydrogen,
halogen,
linear or branched C₁-C₃-alkyl,
linear or branched C₁-C₃-haloalkyl, or
linear or branched C₁-C₃-alkoxy;
B represents one of the following groups (b-1), (b-2) and (b-3); 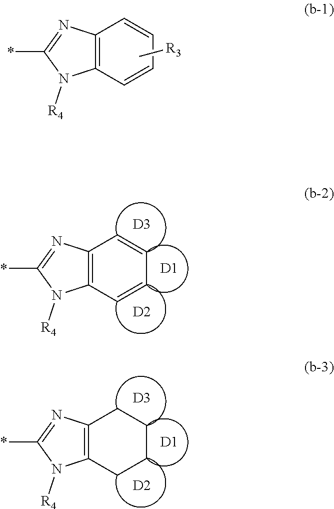
wherein * indicates the binding position;
R³ represents 0, 1, 2 or 3 substituents independently selected from:
linear or branched C₁-C₃-alkyl,
linear or branched C₁-C₃-haloalkyl,
linear or branched C₁-C₃-alkoxy,
unsubstituted or substituted phenyl,
unsubstituted or substituted 5- or 6-membered heteroaryl,
unsubstituted or substituted bicyclic heteroaryl,
6-membered heterocyclyl,
6-membered heterocyclylalkyl,
phenylethinyl, or
pyridinylethinyl,
wherein a substituted phenyl, heteroaryl and bicyclic heteroaryl group can optionally be substituted with 1, 2 or 3 substituents independently selected from:
halogen,
C₁-C₃-alkyl,
C₁-C₃-haloalkyl, and
C₁-C₃-alkoxy;
R⁴ represents
linear or branched C₁-C₆-alkyl,
a dialkylether group [R⁶(CH₂)ₓ—O—CH₂)_{y}—] wherein R⁶ represents a C₁-C₃-alkoxy group and x and y independently represent an integer selected from 1, 2 and 3,
5- or 6-membered unsubstituted heterocyclyl, or
substituted or unsubstituted phenyl, wherein substituents of phenyl are selected from,
halogen, and
C₁-C₃-alkoxy; and
wherein alkyl can optionally be substituted with 1 or 2 substituents, independently selected from;
halogen,
C₁-C₃-alkoxy,
C₆-cycloalkyloxy,
carboxyl,
aminocarbonyl,
mono-alkylaminocarbonyl,
dialkylamino,
3- to 6-membered cycloalkyl,
unsubstituted or substituted 5- or 6-membered heterocyclyl,
unsubstituted or substituted 6-membered aryl,
unsubstituted or substituted 5- or 6-membered heteroaryl, and
unsubstituted or substituted bicyclic heteroaryl,
wherein a substituted heterocyclyl, aryl, heteroaryl and bicyclic heteroaryl group can optionally be substituted with 1, 2 or 3 substituents independently selected from:
halogen,
C₁-C₃-alkyl,
C₁-C₃-haloalkyl,
C₁-C₃-alkoxy,
aminocarbonyl, and
mono-alkylaminocarbonyl,
wherein a mono-alkylaminocarbonyl group may carry a further substituent on the mono-alkyl-chain, selected from halogen-substituted 5- or 6-membered heteroaryl;
and in formulae (b-2) and (b-3) one of D1, D2 and D3 is present and represents;
a fused phenyl ring,
a fused 6-membered heteroaryl ring,
a fused 6-membered cycloalkyl ring, or
a fused 5- or 6-membered heterocyclyl ring;
and the groups (b-2) and (b-3) carry 0 or 1 substituent selected from:
halogen,
linear or branched C₁-C₃-alkyl,
linear or branched C₁-C₃-haloalkyl, and
linear or branched C₁-C₃-alkoxy.

8. The compound or pharmaceutically acceptable salt thereof according to claim 1, represented by one of the formulae (I-C), (I-D), (I-E), (I-F) or (I-G):

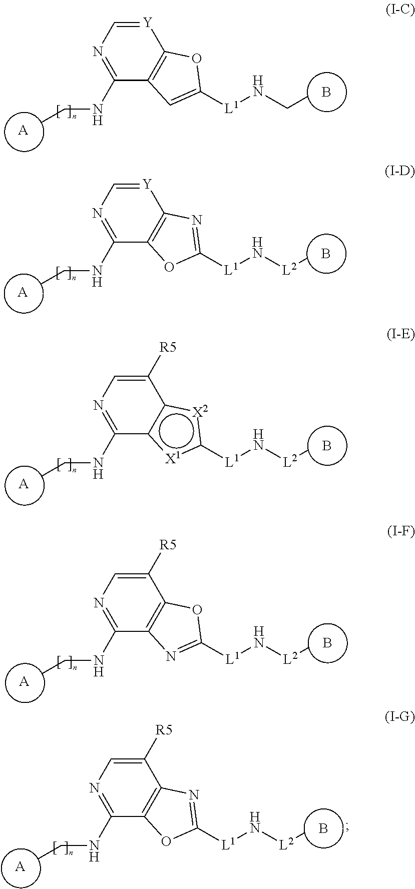

9. The compound or pharmaceutically acceptable salt thereof according to claim 1, wherein one or more of:
the group B is a group (b-1) or (b-2), optionally wherein group (b-2) has one of the following structures: 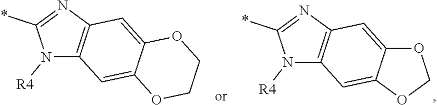
and
the group A has one of the following structures:

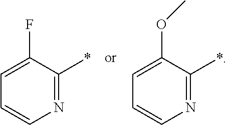

10. The compound or pharmaceutically acceptable salt thereof according to claim 1, wherein one or more of:
halogen substituents are selected from F, Cl and Br;
linear or branched C₁-C₆-alkyl substituents are selected from methyl, ethyl, propyl, iso-propyl, n-butyl and iso-butyl;
C₁-C₃-alkoxy substituents are selected from methoxy and ethoxy;
C₁-C₃-haloalkyl substituents are selected from difluoroethyl (—CH₂—CHF₂) and trifluoromethyl (CF₃);
a substituted alkyl-group in the position R⁴ represents a substituted C₁-C₃-alkyl group; and
a bicyclic heteroaryl group is selected from a benzimidazolyl group.

11. The compound or pharmaceutically acceptable salt thereof according to claim 1, selected from Compound Nos. 1-23:

| No. | Compound/Structure |
|---|---|
| 1 | 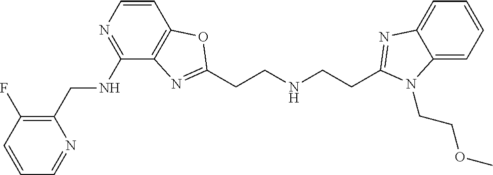 [SMILES: COCCn1c(CCNCCc2nc3c(NCc4ncccc4F)nccc3o2)nc2ccccc21] |
| 2 | 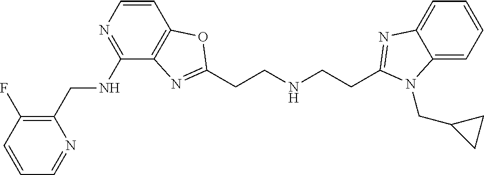 [SMILES: Fc1cccnc1CNc1nccc2oc(CCNCCc3nc4ccccc4n3CC3CC3)nc12] |
| 3 | 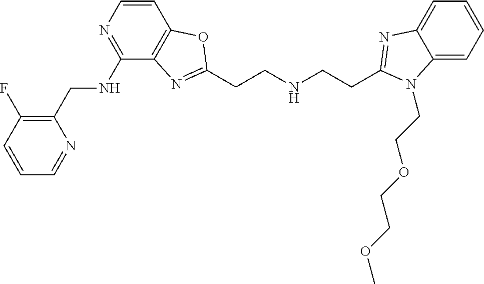 [SMILES: COCCOCCn1c(CCNCCc2nc3c(NCc4ncccc4F)nccc3o2)nc2ccccc21] |
| 4 | 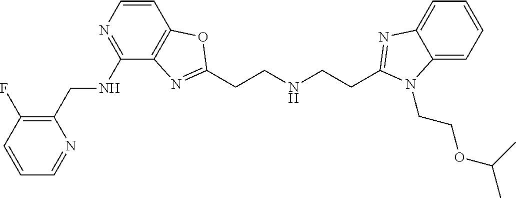 [SMILES: CC(C)OCCn1c(CCNCCc2nc3c(NCc4ncccc4F)nccc3o2)nc2ccccc21] |
| 5 | 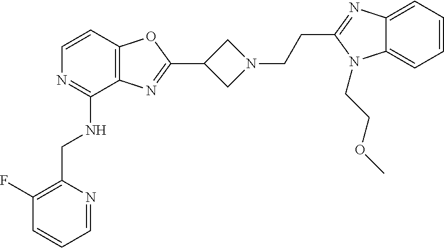 [SMILES: COCCn1c(CCN2CC(c3nc4c(NCc5ncccc5F)nccc4o3)C2)nc2ccccc21] |
| 6 | 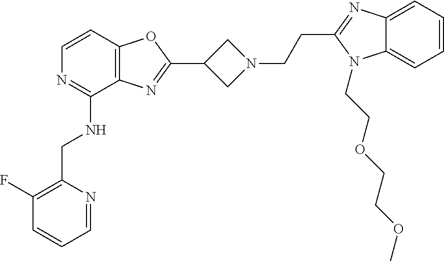 [SMILES: COCCOCCn1c(CCN2CC(c3nc4c(NCc5ncccc5F)nccc4o3)C2)nc2ccccc21] |
| 7 | 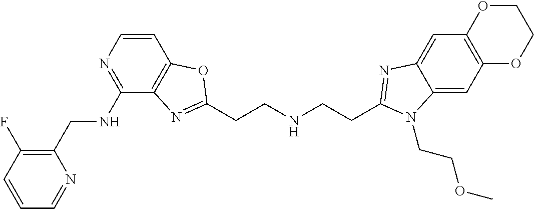 [SMILES: COCCn1c(CCNCCc2nc3c(NCc4ncccc4F)nccc3o2)nc2cc3c(cc21)OCCO3] |
| 8 | 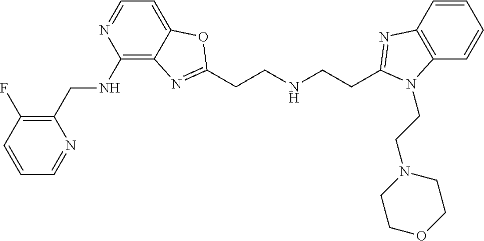 [SMILES: Fc1cccnc1CNc1nccc2oc(CCNCCc3nc4ccccc4n3CCN3CCOCC3)nc12] |
| 9 | 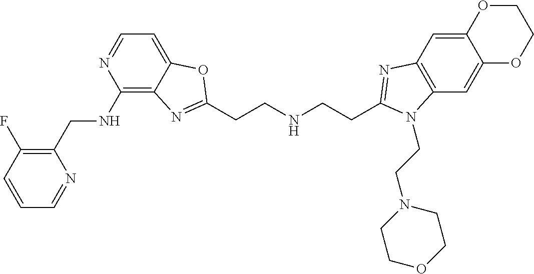 [SMILES: Fc1cccnc1CNc1nccc2oc(CCNCCc3nc4cc5c(cc4n3CCN3CCOCC3)OCCO5)nc12] |
| 10 | 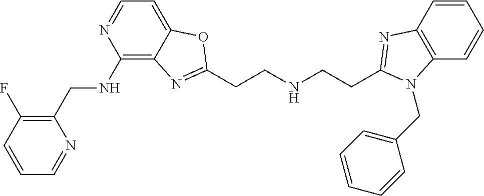 [SMILES: Fc1cccnc1CNc1nccc2oc(CCNCCc3nc4ccccc4n3Cc3ccccc3)nc12] |
| 11 | 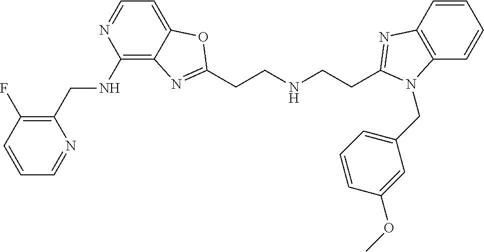 [SMILES: COc1cccc(Cn2c(CCNCCc3nc4c(NCc5ncccc5F)nccc4o3)nc3ccccc32)c1] |
| 12 | 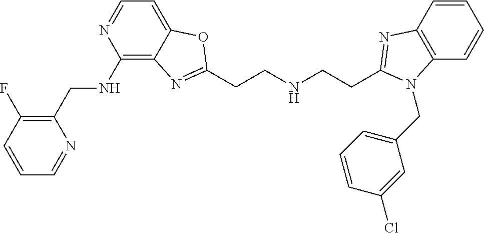 [SMILES: Fc1cccnc1CNc1nccc2oc(CCNCCc3nc4ccccc4n3Cc3cccc(Cl)c3)nc12] |
| 13 | 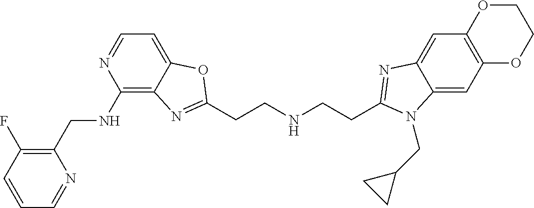 [SMILES: Fc1cccnc1CNc1nccc2oc(CCNCCc3nc4cc5c(cc4n3CC3CC3)OCCO5)nc12] |
| 14 | 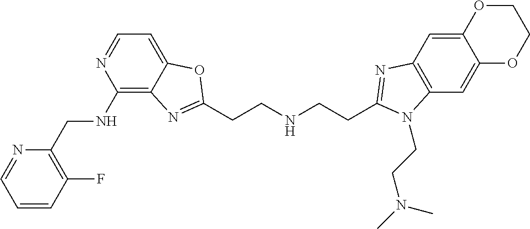 [SMILES: CN(C)CCn1c(CCNCCc2nc3c(NCc4ncccc4F)nccc3o2)nc2cc3c(cc21)OCCO3] |
| 15 | 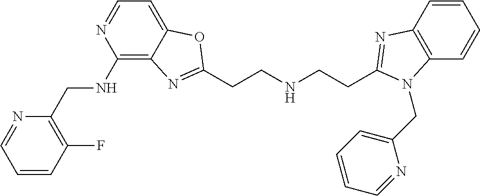 [SMILES: Fc1cccnc1CNc1nccc2oc(CCNCCc3nc4ccccc4n3Cc3ccccn3)nc12] |
| 16 | 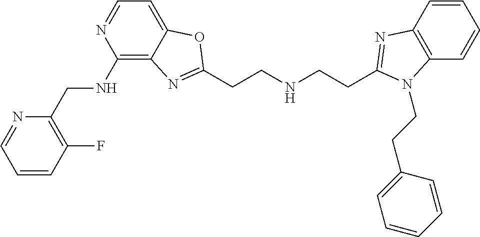 [SMILES: Fc1cccnc1CNc1nccc2oc(CCNCCc3nc4ccccc4n3CCc3ccccc3)nc12] |
| 17 | 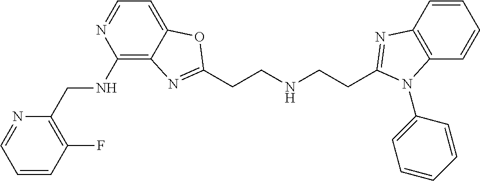 [SMILES: Fc1cccnc1CNc1nccc2oc(CCNCCc3nc4ccccc4n3-c3ccccc3)nc12] |
| 18 | 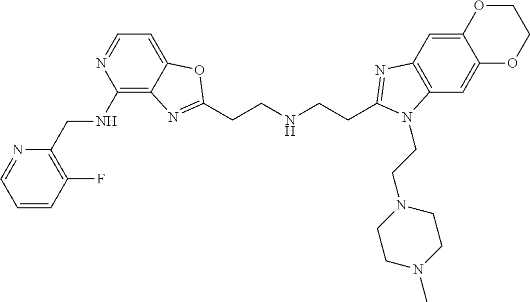 [SMILES: CN1CCN(CCn2c(CCNCCc3nc4c(NCc5ncccc5F)nccc4o3)nc3cc4c(cc32)OCCO4)CC1] |
| 19 | 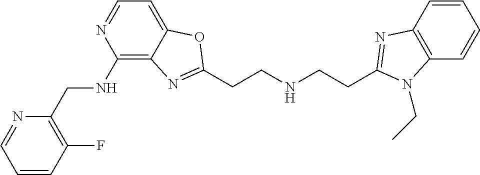 [SMILES: CCn1c(CCNCCc2nc3c(NCc4ncccc4F)nccc3o2)nc2ccccc21] |
| 20 | 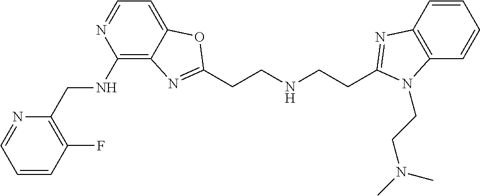 [SMILES: CN(C)CCn1c(CCNCCc2nc3c(NCc4ncccc4F)nccc3o2)nc2ccccc21] |
| 21 | 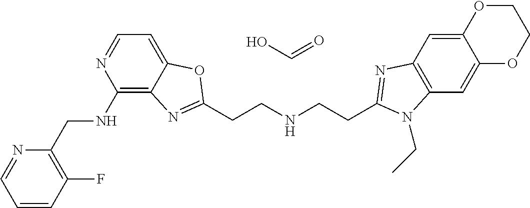 [SMILES: CCn1c(CCNCCc2nc3c(NCc4ncccc4F)nccc3o2)nc2cc3c(cc21)OCCO3 ; O=CO] |
| 22 | 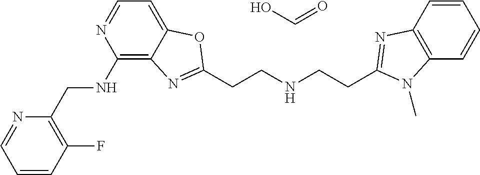 [SMILES: Cn1c(CCNCCc2nc3c(NCc4ncccc4F)nccc3o2)nc2ccccc21 ; O=CO] |
| 23 | 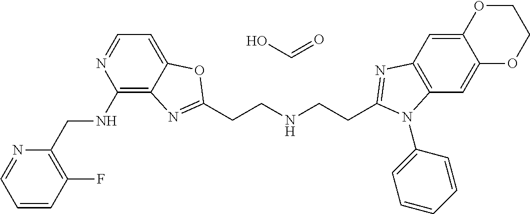 [SMILES: Fc1cccnc1CNc1nccc2oc(CCNCCc3nc4cc5c(cc4n3-c3ccccc3)OCCO5)nc12 ; O=CO] |

12. A hydrate or solvate of the compound or pharmaceutically acceptable salt thereof according to claim 1, or a polymorph of the pharmaceutically acceptable salt, hydrate or solvate.

13. A pharmaceutical composition comprising a compound or pharmaceutically acceptable salt thereof according to claim 1.

14. A method of inhibiting iron transport mediated by ferroportin or a method of prophylaxis treatment of iron metabolism disorders leading to increased iron levels or increased iron absorption, and/or iron overload, wherein the method comprise administering compound or pharmaceutically acceptable salt thereof according to claim 1 to a subject in need thereof.

15. A method selected from:
a method of prophylaxis or treatment of diseases related to or caused by increased iron levels, increased iron absorption or iron overload, selected from thalassemia, including alpha-thalassemia, beta-thalassemia and delta-thalassemia, hemoglobinopathy, hemoglobin E disease, hemoglobin H disease, haemochromatosis, hemolytic anemia, including in particular sickle cell anemia or congenital dyserythropoietic anemia;
a method of prophylaxis or treatment of diseases associated with ineffective erythropoiesis, such as myelodysplastic syndromes (MDS, myelodysplasia), polycythemia vera and congenital dyserythropoietic anemia;
a method of prophylaxis and/or treatment of diseases caused by reduced levels of hepcidin;
a method of prophylaxis or treatment of infections caused by pathogenic microorganisms, such as the bacterium Vibrio vulnificus, in an adjunctive therapy by limiting the amount of iron available to said pathogenic microorganisms;
a method of prophylaxis or treatment of neurodegenerative diseases such as Alzheimer's disease and Parkinson's disease by limiting the deposition or increase of iron in tissue or cells;
a method of prophylaxis or treatment of formation of radicals, reactive oxygen species (ROS) and oxidative stress;
a method of prophylaxis or treatment of cardiac, liver and endocrine damage caused by iron overload; and
a method of prophylaxis treatment of inflammation triggered by excess iron,
wherein the method comprises administering a compound or pharmaceutically acceptable salt thereof according to claim 1 to a subject in need thereof.

16. A pharmaceutical composition containing one or more compounds or pharmaceutically acceptable salts thereof according to claim 1 and one or more of;
one or more compounds selected from pharmaceutical carriers, auxiliaries and solvents, and
at least one additional pharmaceutically active compound selected from active compounds for the prophylaxis and treatment of iron overload, thalassemia, or haemochromatosis, active compounds for the prophylaxis and treatment of neurodegenerative diseases, such as Alzheimer's disease or Parkinson's disease, and the associated symptoms, and iron-chelating compounds.

17. The pharmaceutical composition according to claim 16, which is in the form of a formulation for oral or parenteral administration.

18. A combination therapy, comprising co-administration of compound or pharmaceutically acceptable salt thereof according to claim 1 with at least one additional pharmaceutically active compound, wherein
said co-administration of the combination therapy may be carried out in a fixed dose combination therapy by co-administration of the compound or pharmaceutically acceptable salt thereof according to claim 1 with at least one additional pharmaceutically active compound in a fixed-dose formulation; or
said co-administration of the combination therapy may be carried out in a free dose combination therapy by co-administration of the compound or pharmaceutically acceptable salt thereof according to claim 1 and the at least one additional pharmaceutically active compound in free doses of the respective compounds, either by simultaneous administration of the individual compounds or by sequential use of the individual compounds distributed over a time period; and
wherein the one or more additional pharmaceutically active compounds are preferably active compounds for reducing iron overload, comprising Tmprss6-ASO, iron chelators, curcumin, SSP-004184, Deferitrin, deferasirox, deferoxamine and/or deferiprone; and/or pharmaceutically active compounds which can be selected from antioxidants, such as n-acetyl cysteine; anti-diabetics, such as GLP-1 receptor agonists; antibiotics, such as vancomycin (Van) or tobramycin; drugs for the treatment of malaria; anticancer agents; antifungal drugs; drugs for the treatment of neurodegenerative diseases such as Alzheimer's disease and Parkinson's disease, comprising dopamine agonists such as Levodopa; anti-viral drugs, such as interferon-α or ribavirin; immunosuppressants, such as cyclosporine A or cyclosporine A derivatives; iron supplements; vitamin supplements; red cell production stimulators; anti-inflammatory biologies; anti-thrombolytics; statins; vasopressors; and inotropic compounds.


SEQUENCE LISTING (2 sequences)

SEQ ID NO: 1 - DNA, 21 bp, synthetic construct
  source 1..21: mol_type = other DNA
caggtttgtgagcatcctgaa

SEQ ID NO: 2 - DNA, 19 bp, synthetic construct
  source 1..19: mol_type = other DNA
ggcggcgactaaggagagg